(19) United States
(12) Patent Application Publication
(10) Pub. No.: US 2026/0242713 A1
(43) Pub. Date: Aug. 20, 2026

(54) Apparatus and Method for Performing an In Vitro Study of a Blood Vessel Function

(71) Applicant: Northeastern University, Boston, MA (US)

(72) Inventors: Eno Essien Ebong, Milton, MA (US); Sidi A. Bencherif, Boston, MA (US); Mohammad Hamrangsekachaee, Melrose, MA (US); Ke Wen, Stanford, CA (US)

(21) Appl. No.: 19/181,315

(22) Filed: Apr. 16, 2025

Related U.S. Application Data
(60) Provisional application No. 63/634,853, filed on Apr. 16, 2024.

(51) Int. Cl. C12M 1/12; C12M 1/00; C12M 3/00; G01N 33/50

(52) U.S. Cl. CPC C12M 25/16; C12M 23/20; C12M 23/22; C12M 23/40; C12M 23/46; C12M 29/00; G01N 33/5044

(57) ABSTRACT

Described herein is an apparatus for performing an in vitro study of a blood vessel function. Further described herein is a device configured to emulate pathological or physiological blood vessels as well as a method for using the device in studies of diseases. The apparatus or device may be used for studying various diseases such as blood cardiovascular diseases, infection, inflammation, cancer metastasis or combinations thereof. A kit is also described comprising an apparatus and a substrate.

### RELATED APPLICATION(S)

This application claims the benefit of U.S. Provisional Application No. 63/634,853, filed on Apr. 16, 2024. The entire teachings of the above application(s) are incorporated herein by reference.

### GOVERNMENT SUPPORT

This invention was made with government support under Grant Nos. CMMI 1846962 and 1847843 from the National Science Foundation, and Grant No. EB027705 from the National Institutes of Health. The government has certain rights in the invention.

### SUMMARY

In one embodiment, an apparatus and method for performing an in vitro study of a blood vessel function is an advanced device that can be applied for in vitro study of blood vessel function relevant to complex vascularized tissues and organs. This transparent fluidic device houses mechanically adjustable, non-swelling, endothelial cell adhesion friendly hydrogel substrates that mimic healthy or diseased vascular tissue stiffness conditions, allowing researchers to simulate different cellular environments. The fluidic device presents a flow channel of fixed dimension through which controlled levels of fluid flow are generated and tuned to replicate a variety of real-life vascular flow conditions. The device is transparent for real-time observations. Others have sought to develop similar approaches, primarily using microfluidic devices that have presented some challenges. An example of an advantage of the device described herein, compared to microfluidic counterparts, is its larger size that eliminates common issues like difficulties with cell seeding and nutrient diffusion, and another example is a non-swelling hydrogel that prevents alterations in the flow channel dimensions and resultant flow parameters. Additionally, the apparatus and method for performing an in vitro study of a blood vessel function is versatile, adaptable for a wide range of applications, and can be further customized to suit specific research needs. In summary, the apparatus and method for performing an in vitro study of a blood vessel function, considered with its advantages, is a valuable tool for studying (e.g., assessing or analyzing) vascular biology and mechanobiology processes, investigating vascular disease mechanisms, and conducting drug development research in vitro. Its target market is broad beyond these examples, making it a valuable tool for various research fields.

Described herein is an apparatus for performing an in vitro study of a blood vessel function, the apparatus comprising: a flow chamber structure defining an inlet port, outlet port, flowpath therebetween, and coupling feature; and a hydrogel, seeded with cells, coupled to the coupling feature, the hydrogel, in its coupled arrangement, positioned within the flowpath, a combination of characteristics of the hydrogel and dimensions of the flowpath in a presence of fluid flow creating a pressure between the inlet port and outlet port that causes the apparatus to emulate a blood vessel function. In an embodiment, the flow chamber structure comprises: a flow chamber top substructure defining the inlet port and the outlet port; a flow chamber middle substructure defining at least one window; a flow chamber gasket defining at least one well therein; and a flow chamber bottom substructure; wherein the flow chamber top substructure, flow chamber middle substructure, flow chamber gasket, and flow chamber bottom substructure define respective holes that, in stacked arrangement of the substructures, are aligned; and, in an assembled arrangement, the flow chamber structure produces within the flowpath a uniform laminar flow of fluid during operation with an operating fluid flow pump. In another embodiment, the number of flow chamber middle windows and number of hydrogels are of equal number.

In an embodiment, the cells are embedded in the hydrogel. In another embodiment, the cells are endothelial or epithelial cells. In a further embodiment, the endothelial or epithelial cells produce a layer of glycocalyx (GCX) that cover at least a portion of the hydrogel. In another further embodiment, the flow chamber structure emulates pathological or physiological blood vessels by exposing the layer of GCX to a uniform laminar flow during operation with a fluid flow pump.

In an embodiment, the hydrogel comprises gelatin methacrylate (GelMA) and has an exterior layer of gelatin surrounding the GelMA. In another embodiment, the hydrogel has a stiffness from about 2.5 kPa to about 10 kPa.

In another embodiment, the apparatus further comprises a flow pump configured to pump fluid, the flow pump having an inlet port and an outlet port; and flow tubing having dimensions that enable the flow tubing to couple the outlet port of the flow pump to the inlet port of the flow chamber and the outlet port of the flow chamber to the inlet port of the flow pump, a combination of the flow pump, flow chamber, and flow tubing defining a fluid flow loop that supports the fluid flow. In a further embodiment, the hydrogel comprises gelatin methacrylate (GelMA) and has at least a portion of an exterior layer of gelatin surrounding the GelMA. In another embodiment, the hydrogel has a stiffness from about 2.5 kPa to about 10 kPa. In another embodiment, the apparatus, during operation, emulates a physiological blood vessel function. In another separate embodiment, the apparatus, during operation, emulates a pathological blood vessel function. In another embodiment, the flow pump is configurable to start pumping at an initial shear stress of about 3.6 dyne/cm² and gradually increase the shear stress to a final shear stress of about 12 dyne/cm². In a further embodiment, the endothelial or epithelial cells have an apical surface between the cells and a GCX layer, a basal surface between the cells and the hydrogel, and a junctional surface where the GCX layer is on the hydrogel.

Also described herein is a method for performing an in vitro study of a blood vessel function that retains a hydrogel, seeded with cells, within a flowpath; and causing a fluid to flow in the flowpath to form a fluid flow that interacts with the hydrogel, a combination of characteristics of the hydrogel and dimensions of the flowpath in a presence of the fluid flow creating a pressure that causes the method to emulate a blood vessel function. In a further embodiment, the study enables testing of a disease comprising cardiovascular diseases, infection, inflammation, cancer metastasis, or a combination thereof. In another further embodiment, the cardiovascular disease is atherosclerosis or hypertension. In another further embodiment, the study comprises screening therapeutic agents in the fluid flow for cell targets, treatment for a disease, or a combination of screening for cell targets and treatments for a disease by comparing a control sample and a diseased cell, or sample comprising a diseased cell, or by comparing a cell sample before treatment and after treatment. In a further embodiment, the fluid flow uses a medium comprising cancer cells in the fluid that forms the fluid flow in the flowpath.

Also described herein is a device configured to emulate pathological or physiological blood vessels, the device comprising: a) a flow pump; b) a flow chamber structure defining an input port, output port, flowpath therebetween, and coupling feature; c) a hydrogel, seeded with cells, coupled to the coupling feature, the hydrogel, in its coupled arrangement, positioned within the flowpath, a combination of characteristics of the hydrogel and dimensions of the flowpath in a presence of fluid flow creating a pressure between the input port and output port that emulates a blood vessel function; d) the flow chamber structure comprising: i) a flow chamber top defining an inlet port and an outlet port; ii) a flow chamber middle defining at least one window; iii) a flow chamber gasket defining a at least one well therein; and iv) a flow chamber bottom; v) the hydrogel that fills at least one window of the flow chamber middle that is seeded with endothelial or epithelial cells on the hydrogel or embedded within the hydrogel that produce a layer of GCX that cover the hydrogel and the flow chamber gasket; wherein the flow chamber top, flow chamber middle, flow chamber gasket, and flow chamber bottom each have aligned holes when stacked on top of each other to hold the flow chamber top, middle, gasket, and bottom in place; and when assembled, produces a uniform laminar flow; and e) flow tubing connected to the flow pump and the flow chamber in a loop, wherein the flow tubing has a single directional flow that flows into the inlet port of the flow chamber structure and out of the flow chamber structure through the outlet port of the flow chamber structure, thus emulating a pathological or physiological blood vessels.

In a device embodiment, the hydrogel comprises gelatin methacrylate (GelMA) and has an exterior layer of gelatin surrounding the GelMA. In another embodiment, the hydrogel has a stiffness from about 2.5 kPa to about 10 kPa. In another embodiment, the device is emulating physiological blood vessels. In a different embodiment, the device is emulating pathological blood vessels. In another embodiment, the flow pump starts pumping at to an initial shear stress of about 3.6 dyne/cm² and gradually increases the shear stress to final point of about 12 dyne/cm². In another embodiment, the endothelial or epithelial cells have an apical surface between the cells and the GCX layer, a basal surface between the cells and the hydrogel, and a junctional surface where the produced GCX layer is on the hydrogel.

Also described herein is a method for using the device for in vitro experimentation for testing diseases comprising cardiovascular diseases, infection, inflammation, cancer metastasis, or combinations thereof. In a further embodiment, the cardiovascular diseases are atherosclerosis or hypertension. In another embodiment, in vitro experimentation comprises screening therapeutic agents for cell targets, treatments for the diseases, or a combination of screening for cell targets and treatments for the disease by comparing a control sample and a diseased cell or by comparing a cell sample before treatment and after treatment. In a further embodiment, the device uses media containing cancer cells and therapeutic agents to flow over the GCX layer.

Further described herein is a kit comprising: a) an apparatus for performing an in vitro study of a blood vessel function, the apparatus comprising a flow chamber structure defining an inlet port, outlet port, flowpath therebetween, and coupling feature; and b) a substrate defining features that cause a hydrogel formed thereon to express at least one feature that enables the hydrogel, in a formed state, to couple to the coupling feature in a manner that, in a coupled arrangement, enables the hydrogel to be positioned within the flowpath, a combination of characteristics of the hydrogel and dimensions of the flowpath create a pressure between the inlet port and outlet port that causes the apparatus to emulate a blood vessel function in a presence of fluid flow.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a diagram of a top of an example embodiment of a flow chamber;

FIG. 2 is a diagram of a middle of an example embodiment of the flow chamber;

FIG. 3 is a diagram of a gasket for the embodiment of the flow chamber; and

FIG. 4 is a diagram of a bottom of an example embodiment of the flow chamber.

FIG. 5 is a diagram of an example embodiment depicting (A) Synthesis of GelMA. Gelatin underwent chemical modification to form gelatin methacrylate (GelMA) through a reaction with methacrylic anhydride (MAH) at 55° C. The resulting product was then precipitated using acetone and dried under vacuum. (B) Production of hydrogel. The GelMA solution, containing initiators, was introduced into the gap between the modified glass and the mold. It polymerized to create a hydrogel that is covalently attached to the modified glass. (C) Flow experiment. The resulting hydrogel was used for seeding ECs. After forming a single layer of cells, a 6-hour flow experiment was conducted at a shear stress of 12 dyne/cm². This figure was created using BioRender.com

FIG. 6 is a 1H-NMR spectra for gelatin and GelMA pre-polymerization. (A) Refer to dashed-lined boxes for relevant peaks. Methacryloyl peaks (i.e., vinylic and methyl groups) appeared after the chemical modification of gelatin, while the lysine group was used to quantify the degree of substitution following the chemical reaction. (B) Young's modulus of the hydrogels was measured by a compression test, and 4% (w/v) GelMA hydrogels were considered as physiological substrates, and 10% (w/v) was considered as pathological substrate (n=4, mean±SEM).

FIG. 7 is a diagram of parallel-plates flow chamber design and computational simulation. (A) Three separate plates were used to reduce the possibility of damaging the hydrogels during loading or unloading; where the bottom plate provided a backing surface, the middle surface offered lateral support for the hydrogels, and the top plate and gasket formed the clearance for the fluid to flow. (B) The flow chamber underwent computational simulations. When the flow rate is 215 mL/min, the shear stress along the drawn line is approximately 12 dyne/cm², representing the physiological shear stress. (C) The influence of the 0.5 mm gap is confined to a small area adjacent to the gap. The remaining surface of the hydrogel experiences uniform shear stress.

FIG. 8 is a schematic illustration of the en face and orthogonal views used for analyzing the GCX. Dashed lines correspond to positions of orthogonal views. The composition and concentration of coatings regulate GCX expression on glass slides in the overall schematic.

FIG. 9 demonstrates how stiffness and shear stress influence the formation of actin fibers. Under shear stress, thicker stress fibers formed, suggesting a stronger attachment to the surface. In softer samples, there were more peripheral actin fibers, which are indicators of physiological conditions. However, in ECs on stiffer substrates, the presence of stronger stress fibers and fewer peripheral fibers could potentially lead to EC dysfunction. This data confirms the effectiveness of the presented system in modulating EC behavior.

FIG. 10 is a schematic illustration of the interaction between actin and YAP, showing that actin polymerization leads to YAP dephosphorylation and subsequent nuclear translocation.

FIG. 11 is (A) Microscopic images showing HS (green) expression in ECs cultured in conditions of shear stress (12 dyne/cm²) combined with substrate stiffness of 5 kPa or 10 kPa. Blue indicates DAPI (EC nuclei marker). Scale bar=10 μm. (B) Quantification of normalized HS MFI, derived from en-face microscopic views of ECs conditioned under flow, comparing substrates of 5 kPa versus 10 kPa. (C) Percentage area of EC surfaces covered by HS under various conditions (statistical significance was determined using Student's t-test; *p<0.05). (D) Orthogonal views of EC monolayers under flow conditions on substrates with 5 kPa versus 10 kPa stiffnesses. These views illustrate HS thickness and the presence of junctional, apical, and basal HS. Scale bar=10 μm. (E) Schematic illustration of the methodology used for compartmentalizing thickness measurements into junctional, apical, and basal categories.

FIG. 12 is microscopic images showing the impact of shear stress (12 dyne/cm²) with substrate stiffness of 5 kPa versus 10 kPa on SA (green) expression in ECs. Blue indicates DAPI (EC nuclei marker). Scale bar=10 μm.

FIG. 13 is a graphical representation of the hypothesis concerning the implications of reduced SA thickness in the junctional region.

FIG. 14 is a potential interaction between GCX and YAP with a graphical representation of the hypothesis suggesting that CD44 expression may activate YAP through the CD44-RhoA-YAP pathway.

FIG. 15 is a schematic of various conditions including sialic acid, heparan sulfate, and YAP and CD44 compared to an example embodiment with the in vitro model system.

The foregoing will be apparent from the following more particular description of example embodiments, as illustrated in drawings being filed herewith and in drawings interspersed in the manuscripts being filed herewith. The drawings are not necessarily to scale, emphasis instead being placed upon illustrating embodiments.

### DETAILED DESCRIPTION

A description of example embodiments follows.

An example embodiment comprises gelatin methacrylate (GelMA) hydrogel synthesized by dissolving type A, 300 bloom gelatin from porcine skin in a carbonate-bicarbonate buffer, followed by methacrylation with methacrylic anhydride (MAH). The resulting GelMA solution was precipitated in acetone, dried, and stored for further use. Glass slides were then functionalized with 3-(Trimethoxysilyl)propyl methacrylate to enable covalent attachment of hydrogels during polymerization. Hydrogels were prepared using a redoxinduced free radical polymerization method involving GelMA, tetramethylethylenediamine (TEMED), and ammonium persulfate (APS), followed by cross-linking in Teflon molds and equilibration in phosphate buffered saline (PBS) at 37° C. Additionally, the hydrogels were coated with gelatin. These methods aimed to fabricate mechanically and chemically tunable hydrogels conducive to studying adherent endothelial cell monolayers' role in vascular health, atherosclerosis, hypertension, and other conditions. In some embodiments, a leak-proof enclosure was meticulously designed and validated using SolidWorks® computer aided design software and SolidWorks® Flow Simulation software. Acrylic was chosen as the primary material to ensure transparency of the enclosure. It comprises several components, including the flow chamber bottom, flow chamber middle, flow chamber gasket, and flow chamber top, with detailed engineering drawings and schematics being filed herewith. To prepare the device for use, cells are cultured on the hydrogels and/or embedded within them, depending on the research goals. For instance, endothelial cells can be cultured on the hydrogel surface. Once cells reach the desired confluency, the hydrogels are placed into designated wells, and the chamber is assembled using screws to secure the top to the bottom, with the gasket ensuring a sealed enclosure and creating the flow channel. Finally, the chamber is connected to a tube loop via inlet and outlet ports on the chamber top, requiring connection to a pump for fluid flow, with options including peristaltic or centrifugal pumps depending on specific requirements and resources.

An example embodiment of the apparatus offers a significant advantage by (i) providing a platform for exposing cultured vascular cells to combined fluid and stiffness mechanical stimuli, replicating both healthy and diseased mechanical environments for comprehensive research. Additional benefits include: (ii) simplified cell plating on or within the hydrogel outside of the fluidic device, ensuring consistency and streamlining processes; (iii) its larger size facilitates proper nutrient and oxygen diffusion, overcoming limitations of smaller microfluidic alternatives; (iv) compatibility with standard microscope stages enables live imaging and real-time data collection on flow patterns and cellular responses to mechanical stimuli.

An example embodiment offers a distinct performance edge compared to similar apparatuses/devices available on the market, due to several advantages. While serving as a flow chamber the apparatus also accommodates hydrogels with varying stiffness levels, which allows for accurate modeling of healthy versus disease environments and subsequent cell experimentation. Other apparatuses/devices lack control over substrate stiffness. The hydrogel-based design also resists swelling, maintaining stability during experiments, while competing devices grapple with unpredictable swelling that affects reliability and reproducibility. In some embodiments of the apparatus allow for the incorporation of immobilized chemical cues directly into the hydrogel surface, ensuring optimal cell growth conditions. Furthermore, cells can be seeded onto the substrate outside the enclosure, ensuring a more controlled initial environment. Precision seeding optimizes subsequent fluid flow experiments.

An embodiment may be used to model and study the large vessel function in vitro using macrovascular endothelial cells, smooth muscle cells, and fibroblasts, or study small vessel function in vitro using microvascular endothelial cells with pericytes, contributing to a comprehensive understanding of vascular dynamics. In example embodiments, these cells may be seeded in the hydrogel-based design either by seeding the cells on the surface of the hydrogel, or by mixing the cells with hydrogel mixture prior to forming a hydrogel to embed the cells within the hydrogel. Methods of embedding smooth muscle cells are known in the art (see, e.g., https://pubs.acs.org/doi/10.1021/acsami.3c08511, DOI: 10.34133/bmr.0076, the contents of which are incorporated herein their entirety herein).

An embodiment may also be used to model and study ailments and diseases, such as inflammation (e.g., by incorporating immune cells in the flow stream), atherosclerosis, hypertension (through using stiffer hydrogels), infection, cancer metastasis (e.g., by incorporating circulating tumor cells), or other conditions.

In another exemplary use of an embodiment may be to screen therapeutic agents to aid in the development of effective vascular cell targets and vascular treatments.

The disclosed technology is very promising for various commercial applications that require advanced cell culture models. In some embodiments, this technology as an in vitro model that accurately mimics the mechanical environment of the vasculature and can be applied to study blood vessel function.

In some embodiments, this technology as an in vitro model that accurately mimics the mechanical environment of the vasculature and can be applied to screen endothelium and vascular targeting drugs. Current methods for evaluating endothelium and vascular targeting drug efficacy are expensive or lack the complexity of in vivo models.

In some embodiments, hydrogels are used that specify the use of gelatin, gelatin methacrylate (GelMA), or combinations thereof. Additional or alternative materials may be used as well, including but not limited to hydrogels that have a tunable rigidity and promote cell attachment, as well as low-to-non-swelling hydrogels that promote cell adhesion. In some non-limiting embodiments, modified polyacrylamide may be used in the hydrogel. Further, any of the hydrogel candidates may be used in combination with each other, including layering, i.e., a GelMA hydrogel with a gelatin layer.

In further, non-limiting embodiments, the synthesis of the hydrogel, more so relating to a GelMA hydrogel, may use alternative organic solvents in the process of synthesizing the GelMA hydrogel. Some non-limiting organic solvents including: acetone, ethanol, methanol, isopropanol, and other organic alcohols may be used in the precipitation process of forming a GelMA hydrogel.

In some embodiments, cells may be obtained from a human patient or subject, a non-human patient or subject, or cultured from a cell line.

In some embodiments, the term “couple to” refers to coupling of a substrate to a coupling feature which may include press fit onto extender (e.g., barbs) of a coupling feature, be gripped by arms of a coupling feature, be contained by at leasts one strapping defined by the coupling feature, or other form of coupling technique for maintaining physical coupling between a coupling feature and a substrate in the presence of a fluid flow known in the art.

As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., human patients, being treated. Many medical treatments are effective for some, but not all, patients that undergo the treatment.

In some embodiments, a treatment study may include adding a therapeutic agent to the apparatus of the embodiments. In some other embodiments, a treatment study may include culturing cells in the apparatus from a diseased patient and then comparing the diseased cells to the cells after a therapeutic treatment or multiple therapeutic treatments with a therapeutic agent.

In some embodiments, “therapeutic agents” may refer to a pharmaceutical compound that alleviates or treats a disease.

As used herein a “control sample” may indicate a control or “normal” (e.g., undiseased) cells.

As used herein, the terms “apparatus” and “device” are used interchangeably herein. As used herein, a method of performing a study (i.e., a method of studying) is described herein.

### Example 1

Glycocalyx (GCX) is a carbohydrate-rich structure that coats the surface of endothelial cells (ECs) and lines the blood vessel lumen. Mechanical perturbations in the vascular environment, such as blood vessel stiffness, can be transduced and sent to ECs through mechanosensors such as GCX. Adverse stiffness alters GCX-mediated mechanotransduction and leads to EC dysfunction and eventually atherosclerotic cardiovascular diseases. To understand GCX regulated mechanotransduction events, an in vitro model emulating in vivo vessel conditions is needed. Example 1 investigated the impact of matrix chemical and mechanical properties on GCX expression via fabricating a tunable non-swelling matrix based on the collagen-derived polypeptide, gelatin. To study the effect of matrix composition, Example 1 conducted a comparative analysis of GCX expression using different concentrations (60-25,000 μg/mL) of gelatin and gelatin methacrylate (GelMA) in comparison to fibronectin (60 μg/mL), a standard coating material for GCX-related studies. Using immunocytochemistry analysis, Example 1 showed for the first time that different substrate compositions and concentrations altered the overall GCX expression on human umbilical vein ECs (HUVECs). Subsequently, GelMA hydrogels were fabricated with stiffnesses of 2.5 and 5 kPa, representing healthy vessel tissues, and 10 kPa, corresponding to diseased vessel tissues. Immunocytochemistry analysis showed that on hydrogels with different levels of stiffness, the GCX expression in HUVECs remained unchanged, while its major polysaccharide components exhibited dysregulation in distinct patterns. For example, there was a significant decrease in heparan sulfate expression on pathological substrates (10 kPa), while sialic acid expression increased with increased matrix stiffness. This study suggests the specific mechanisms through which GCX may influence ECs in modulating barrier function, immune cell adhesion, and mechanotransduction function under distinct chemical and mechanical conditions of both healthy and diseased substrates.

Atherosclerosis, the underlying cause of cardiovascular diseases, accounts for 37% of deaths in individuals up to 70 years old (Virani et al., 2021). Atherosclerosis is a medical condition that involves the narrowing of the vessel lumen, leading to an increase in flow resistance, as well as the thickening and hardening of the vessel wall (Laroia et al., 2003; Kohn et al., 2015). Severe atherosclerosis is usually associated with increased blood vessel rigidity that occurs with age and in cases of hypertension (Tegos et al., 2001; Benetos et al., 2002; Mitchell et al., 2010).

The endothelium lining the vessel luminal surface is critical to vascular health. Endothelial cells (ECs) play a role in regulating various cardiovascular functions, such as vessel tone regulation, selective permeability, hemostasis, and mechanotransduction. EC dysfunction is widely suggested as a primary contributor to the development of atherosclerosis (Deanfield et al., 2007; Suowen et al., 2021). Since ECs are exposed to different mechanical stimuli at the interfaces between ECs and blood flow, as well as between ECs and underlying vessel tissues (Tarbell and Pahakis, 2006; Jansen et al., 2017), numerous studies have attributed EC dysfunction with abnormal alterations in mechanical cues within the vascular environment. These cues include fluid shear stress derived from blood and tissue stiffness (Topper et al., 1996; Bonetti et al., 2003; Mitchell et al., 2010). Considering the attention given to the impact of shear stress on cellular responses, herein the focus is on stiffness. In healthy blood vessels, the substrate stiffness underlining ECs typically ranges from 2.5 to 5 kPa. This range may vary based on the measuring methods, including non-invasive techniques like pulse wave velocity (PWV), ultrasound, and magnetic resonance imaging (MRI) for in vivo and ex vivo atomic force microscopy (AFM) (Engler et al., 2004; Klein et al., 2009; Peloquin et al., 2011; Stroka and Aranda-Espinoza, 2011; Lee et al., 2017). In pathological conditions, the substrate undergoes remodeling, and becomes stiffened (stiffness >10 kPa) (Peloquin et al., 2011). These changes typically arise due to abnormal alterations in substrate composition. For instance, the loss of collagen, whose primary role is to provide the main tensile strength of the artery wall, can elevate the risks associated with foam-cell macrophage activation (Newby, 2008). In order to reduce the mortality associated with atherosclerosis, it is crucial to elucidate the underlying factors and mechanistic causes of EC dysfunction that originate from the substrate.

One of the key regulators of EC function is glycocalyx (GCX), which is a multifunctional layer that covers vascular ECs. GCX primarily consists of proteoglycans, glycoproteins, and glycosaminoglycan (GAG) chains. Proteoglycans are present as core proteins bound to the cell membrane with GAGs attached to them (Tarbell and Cancel, 2016; Zeng et al., 2018). Studying GCX has been challenging due to its complex and delicate structure; however, the evidence demonstrating its influence on EC function is steadily increasing. As a mechanotransducer, GCX has been extensively reported for its ability to sense fluid shear stress within its surrounding microenvironment. This, in turn, mediates flow-induced activation of endothelial NO synthase (eNOS), as well as the expression of adhesion molecules and dysregulation of inflammatory genes (Ebong et al., 2010; Yen et al., 2015; Hamrangsekachaee et al., 2022). In contrast to the shear stress, the impact of the substrate matrix, especially the solid-derived forces originating from it, has received less attention in research. Recent studies have provided intriguing evidence regarding the effect of substrate stiffness on GCX expression (Mahmoud et al., 2021a; Mahmoud et al., 2021b). The potential effect of substrate chemistry on GCX expression remains unknown. Therefore, further research is necessary to elucidate the role of mechanical and chemical properties of the substrate matrix on GCX expression and, subsequently, on EC function.

Example 1 is aimed to investigate GCX chemical sensitivity to the substrate matrix material. Gelatin methacrylate (GelMA) derived from collagen was synthesized as a physiologically relevant matrix. GelMA was chosen as the material of preference due to its advantageous characteristics. These include its high biocompatibility, the presence of adhesive molecules like arginine-glycine-aspartic acid (RGD), and the ability to fine-tune its mechanical properties. Additionally, GelMA allows for easy and cost-effective hydrogel fabrication, thanks to its versatility in crosslinking methods, such as light-based techniques and redoxinduced polymerization. Furthermore, GelMA exhibits the capability to encapsulate various types of cells and bioactive molecules. This versatility makes it a material of choice for the subsequent development of the model, facilitating the inclusion of vascular smooth muscle cells (VSMCs) to enrich the model's complexity and physiological relevance (Van Den Bulcke et al., 2000; Yue et al., 2015; Lavrentieva et al., 2020; Cuvellier et al., 2021).

Another objective was to investigate GCX chemical sensitivity to different substrate coatings. It is worth noting that fibronectin is commonly used as a substrate coating in atherosclerosis studies to improve cell attachment (Stroka and Aranda-Espinoza, 2011; Yeh et al., 2012; Mahmoud et al., 2021a; Mahmoud et al., 2021b). Many informative studies using fibronectin as a substrate for cultured ECs have been performed (Ebong et al., 2011; Ebong et al., 2014; Mensah et al., 2017; Harding et al., 2018; Mensah et al., 2020). However, excessive deposition of fibronectin by ECs has been observed under adverse shear stress conditions in animal models of cardiovascular disease. The deposition occurs at early stages of atherosclerosis, preceding deposition of fibrinogen which typically occurs later (Hahn et al., 2009; Hamrangsekachaee et al., 2022). Moreover, Wayne Orr et al. demonstrated that fibronectin coating, compared to collagen I coating, upregulates atherogenic genes. Notably, the observed increased expression levels of intercellular adhesion molecule 1 (ICAM-1), vascular cell adhesion molecule 1(VCAM-1), and Nuclear factor-κB (NF-κB) (Orr et al., 2005; Chirinos, 2012). Therefore, in the present study it was hypothesized that the composition of the substrate coating could potentially contribute to dysregulation in GCX expression. To test this hypothesis, first glass slides were coated with fibronectin, gelatin, and GelMA at a concentration of 60 μg/mL (this is considered as baseline concentration, or 1× concentrated). In light of the fact that the hydrogels would be composed of GelMA and given that GelMA could be tuned to vary the concentration of adhesive RGD molecules, experiments were also performed to further assess GCX sensitivity to different concentrations (greater than 60 μg/mL) of GelMA coating. For the various coating models to more accurately mimic healthy and diseased conditions. The specific interest of the present study is to understand the impact of substrate matrix mechanical properties on GCX expression on ECs, requiring the fabrication of a reproduceable, mechanically, and chemically tunable EC-compatible hydrogel. To fulfill this requirement, GelMA synthesis was carried out. First, type A, 300 bloom gelatin from porcine skin was dissolved in 0.25M carbonate-bicarbonate buffer at a concentration of 10% (w/v) at 55° C. The pH of the gelatin solution was adjusted to 9.5 using 0.1M NaOH. MAH was added to the solution in a volume ratio of 1:100 to start the methacrylation reaction. The solution was stirred at 500 RPM for an hour to complete the reaction (Zhu et al., 2019). The resulting GelMA solution was then added dropwise to an excess acetone solution at 200 RPM to cause precipitation (Kim et al., 2014). The product was collected on absorbent papers, dried in a vacuum oven at room temperature (RT), and stored at −20° C. until further use. To synthesize FITC-labeled gelatin or GelMA, the previously established protocol was followed (Rezaeeyazdi et al., 2018). Briefly, gelatin or GelMA was dissolved in a sodium bicarbonate solution at a concentration of 10% (w/v), and the pH was adjusted to 8.5. NHS-FITC was added to the solution at a concentration of 0.01% (w/v), and the mixture was stirred at 500 RPM overnight. FITC-labeled gelatin GelMA was obtained by precipitating the solution in excess acetone and then dried in a vacuum oven at RT.

### Chemical Characterization of GelMA

GelMA was characterized to evaluate the degree of substitution. Therefore, proton nuclear magnetic resonance (¹H NMR) spectra were obtained using Varian Inova-500 NMR and Brucker 500 MHz NMR spectrometers. GelMA was dissolved in D₂O at a concentration of 2 mg/mL and the spectrum was acquired at RT, with a 15 Hz sample spinning at a 45° angle and an 8 μs delay for 128 scans. The methacryloyl peak areas were integrated at 5.4 and 5.6 ppm. The degree of substitution was determined by calculating the ratio of the number of amine groups to the gelatin amines prior to the methacrylation reaction. The aromatic region was used as a control for the concentration of gelatin and GelMA (Rezaeeyazdi et al., 2018).

To assess the consumption of methacrylate groups during polymerization, the hydrogels were washed, lyophilized for 3 days, snap frozen, crushed, and suspended in D₂O for ¹H NMR. The disappearance of vinylic peaks was used as evidence for the consumption of the methacryloyl residues.

### Glass Coating

To enhance the handling and fabrication of hydrogels, plain microscope slides (1 mm thickness) were precisely cut into 2.5×2.5 cm² pieces using a FlipScribe glass cutter (LatticeGear). The cut slides were thoroughly washed with soap, sonicated for 5 min in ethanol, and air-dried. Subsequently, the slides were functionalized with 3-(Trimethoxysilyl)propyl methacrylate to improve their surface properties and allow the covalent attachment of hydrogels during the polymerization process. A 0.5% solution of 3-(Trimethoxysilyl)propyl methacrylate in absolute ethanol was prepared, and 3% dilute glacial acetic acid solution (10% solution in deionized (DI) water) was added to the mixture. The resulting solution was poured over the glass slides and allowed to react for approximately 5 min on a rocker (80 RPM). After completion of the reaction, the slides were washed with ethanol 2-3 times and stored in the dark at RT.

### Hydrogel Preparation

Hydrogels were synthesized using a redox-induced free radical polymerization method. GelMA was dissolved in DI water at various concentrations and heated to 45° C. TEMED (12 mM for 2.5 kPa hydrogel, 24 mM for 5 kPa hydrogel, and 6.25 mM for 10 kPa hydrogel) and APS (12 mM for 2.5 kPa hydrogel, 24 mM for 5 kPa hydrogel, and 24.5 mM for 10 kPa hydrogel) were added to the GelMA solution and mixed thoroughly. The resulting solution was quickly poured into Teflon molds at RT and allowed to crosslink. After completion of the reaction, the hydrogels were transferred to 1×PBS and equilibrated at 37° C. to remove the remaining reactants and byproducts.

### Mechanical Compression and Rheological Testing

To select the substrates emulating the physiological and pathological matrix stiffnesses, the mechanical tests were conducted using a TA Electroforce 5,500 mechanical loading device (TA Instruments, New Castle, United States) with a calibrated 1,000 lb load cell at RT in an aqueous environment. Cylindrical samples with dimensions of 8×4 cm² were inserted between the compression plates. The compression plate was lowered at a rate of 0.01 mm/s until a total displacement of 50% of the sample height was achieved. Data for load and displacement were recorded and used to calculate stress and strain. The slope of the stress-strain curve between 5% and 10% strain was used to determine the Young's modulus. The polymerization time and rheology of GelMA hydrogels were evaluated using an ARES RFS 3 rheometer with stainless-steel cone and plate (cone angle of 0.0403 rad, gap size of 0.0508 mm, 25 mm). To determine the linear viscoelastic region of the hydrogel, a strain sweep test was performed at a frequency of 10 rad/s with a strain range of 0.1%-100%. The linear viscoelastic region was determined by plotting the storage modulus (G′) against shear strain (y %) on a log-log plot and identifying the point at which G′ exhibited strain-dependent behavior. A time sweep test was carried out at a frequency of 10 rad/s and a strain of 3% to determine the gelation point, which was reported when G′ and G″ intersected at a phase angle of 45°, indicating the onset of gel formation.

### Swelling Behavior of Hydrogels

The swelling behavior of the hydrogels was quantified by calculating the ratio of the weight of the swollen hydrogel to the weight of the dehydrated gel after 48 h of lyophilization. Additionally, to assess the dimensional changes resulting from swelling up to equilibrium, the hydrogels were incubated perpendicularly in PBS at 37° C. overnight. Microscopy images of the hydrogels were captured using an AXIO observer Z1 microscope (Cal Zeiss Meditec AG), and their thicknesses were measured.

### Endothelial Cell Culture

To investigate the effect of substrate chemical and mechanical properties on GCX expression, HUVEC were used. The HUVECs were cultured in vascular cell basal medium supplied with EC growth kit containing various growth factors: recombinant human vascular endothelial growth factor (rh-VEGF: 5 ng/mL), epidermal growth factor (rh-EGF: 5 ng/mL), basic fibroblast growth factor (rh-FGF basic: 5 ng/mL), insulin-like growth factor-1 (rh-IGF-1:15 ng/mL), L-glutamine (10 mM), heparan sulfate (0.75 Units/mL), hydrocortisone (1 μg/mL), ascorbic acid (50 μg/mL), fetal bovine serum (2% FBS), and 1% penicillin/streptomycin. The cells were incubated in a humidified incubator at 37° C. with 5% CO₂. For the experiments, passage 4 to 7 HUVECs were used, consistent with the previously published studies (Mensah et al., 2020). To study the effect of composition on GCX expression, No. 1.5 microscope cover glasses were prepared for the experiments. The cover glasses were sterilized either by autoclaving or by treating them with 70% ethanol, and then washed with PBS. The coating materials were prepared at an initial concentration of 60 μg/mL, referred to as 1×. Human fibronectin was used as the control at 1× concentration. Gelatin and GelMA were used at different concentrations: 1×, 5×, 10×, 100×, and 400×. 400× is the highest practical concentration close to that of GelMA solution used for fabricating hydrogels with a stiffness of 2.5 kPa. The coating solutions were applied to cover glasses and incubated at 37° C. for 45 min. Afterward, the cover glasses were rinsed with PBS. HUVECs were seeded on the coated surfaces at a density of 5,000 cell/cm2. The media was changed every other day until the HUVECs reached full confluency. Prior to the experimental endpoint, 0.5% BSA was added to media overnight to enhance GCX stability. To investigate the effect of stiffness, hydrogels were prepared as described in Section 2.4 and cut into 2.5×2.5×0.1 cm3 dimensions. The hydrogels were then incubated in PBS at 37° C. overnight. Subsequently, the hydrogels were sterilized using 70% ethanol on a rocker for 45 min. Following sterilization, the hydrogels were treated with 1× gelatin for 45 min at 37° C. and incubated in cell culture media overnight prior to cell seeding. HUVECs showed a lower cell attachment to the hydrogels in comparison to the glass slides. Therefore, ECs were seeded on the hydrogels at a higher density of 100,000 cell/cm², and the media was changed every 2 days until the cell layer reached 100% confluency occurring after 3-4 days. To stabilize GCX, 0.5% BSA was added to media overnight before fixation.

### Histological Analysis

To study GCX response to substrate matrix conditions, the GCX and its major polysaccharide components, including heparan sulfate (HS), hyaluronic acid (HA), and sialic acid (SA) were labeled. Once the monolayer of ECs reached 100% confluency (after 3-4 days), a wash with 1% BSA was performed, followed by fixation. A fixative solution containing 2% paraformaldehyde and 0.5% glutaraldehyde in PBS was used for 30 min to prepare the samples for histology. For saccharide, a fixative solution of 4% paraformaldehyde was used for 15 min. The fixation was done at RT before applying blocking agents (BSA or goat serum) for 1 h. Lectins were used to bind to sugar moieties for labeling GCX and SA. The samples were incubated with biotinylated WGA and biotinylated elderberry bark lectin, respectively, at a dilution of 1:100 each for 1 hour at RT. Secondary labeling was carried out by incubating the samples with Alexa flour 488 (AF488)-conjugated streptavidin at a dilution of 1:1,000, for 1 hour at RT. For HS and HA labeling, the samples were transferred to humid chambers and incubated with clone F58-10E4 antibody against HS and biotinylated HA binding protein, respectively, at a dilution of 1:100 each for 3 days at 4° C. Secondary labeling for HS and HA was performed by incubating the samples with AF488-conjugated goat anti-mouse IgM at a dilution of 1:400 and AF488-conjugated streptavidin at a dilution 1:50, respectively, for 1 h. Finally, the samples were rinsed and mounted using DAPI-containing mounting media. Negative control studies were performed to assess the lectin, binding protein, and antibody specificity and extract non-specific staining data to be subtracted from histology data of GCX sensitivity to substrate chemistry and stiffness.

### Imaging and Image Analysis

Z-stack images were captured using Zeiss LSM 800 and 710 confocal microscopes (Cal Zeiss Meditec AG) at ×63 (oil emission) magnification. Further imaging parameters are detailed in Table 1. GCX expression on HUVECs, including both GCX and its components, was quantified using multiple methods: 1) normalized GCX thickness, measured in the orthogonal direction; 2) normalized GCX component expression, determined by normalizing mean fluorescence intensity (MFI) measurements, which indicate aggregate density, in the en face view; and 3) percent GCX coverage of the EC apical surface, also determined in the en face view. These methods are based on well-established GCX analysis approaches. This being said, it is understood in the GCX research community that measured GCX thickness, expression, and coverage values may be influenced by various factors, including microscope resolution, occasional internalization of labeling agents, and fusion of apical and basal signals. Consequently, in this paper, GCX measurements are not always shown as absolute but may be normalized to show relative GCX differences when comparing the impact of various conditions on the GCX.

TABLE 1
Parameters used for confocal microscopy

| | | Laser | Detector | Z-stack |
|---|---|---|---|---|
| | | Power | Gain | Distance |
| Marker | Microscope | (%) | (V) | (μm) |
| WGA (On | Zeiss LSM 800 | 0.4 | 625 | 0.19 |
| glass slides) | | | | |
| WGA | Zeiss LSM 800 | 0.4 | 625 | 0.19 |
| (Hydrogel) | | | | |
| HS | Zeiss LSM 710 | 2.4 | 750 | 0.38 |
| SA* | Zeiss LSM 710 | 2.5 | 800 | 0.38 |
| SA* | Zeiss LSM 800 | 0.2 | 550 | 0.38 |
| HA | Zeiss LSM 800 | 0.1 | 500 | 0.38 |

*Two microscopes were utilized for SA imaging, the data was normalized to 2.5 kPa samples obtained from the same microscopes to remove the variation among microscopes.

For GCX thickness quantification, a custom Python program was developed. The thickness of GCX in the X-Z dimension was estimated from the GFP (AF488) channel, which captured the GCX intensity at a wavelength of 488 nm. To reduce image noise and refine the details, a Gaussian blur filter was first applied to the sample image. Then, the image was thresholded with the Otsu method, which divides the pixels into two classes based on intensity histogram and separates the foreground GFP (AF488) fluorescence regions of interest (ROI) from the background. The algorithm proceeded with randomly drawing a vertical line within the ROI along the X-axis and counting the number of pixels (or z-stack layers) that had fluorescence intensity greater than the threshold on the line. Multiplying this count by the length of pixels (or the intervals between z-stack layers) provided the ROI thickness, which represented the GCX thickness. The process was repeated for a total of 50 lines, and the average of their thickness values was calculated as the average GCX thickness expressed on the sample image.

For quantifying the normalized GCX component expression, a different Python algorithm was developed. This algorithm measured average GFP (AF488) intensity expressed in every pixel of the image in the en face view, which represented the sample Mean Fluorescence Intensity (MFI). MFI was taken as an indicator of the aggregate density of the expressed GCX components that were under examination. The data then were normalized with respect to a reference group in each figure by dividing the intensity values by the mean intensity of the reference group.

To quantify the percentage of GCX coverage of the EC apical surface area, indicating the distribution of GCX components, CellProfiler 4.2.4 (Broad Institute, MA) was utilized. However, for this particular study, only the quantification of HS, SA, and HA is reported. Measurements of percent area covered by GCX (WGA-labeled GCX) on ECs were obtained but are not included in this report due to the observation of a nearly complete area coverage of GCX across all the samples.

All custom-designed Python modules used for analyzing the captured confocal images in both the orthogonal and en face dimensions, as well as for quantifying the GCX, are publicly available online at https://github.com/KE-Chloe-WEN/GCX_quant.

### Statistical Analysis

In the context of this study, the term “N” is used to denote biological replicates, which refers to an independent set of experiments conducted to validate the results and ensure the reliability of the findings. On the other hand, “n” represents operational replicates, which entails repetitions of specific treatments or interventions within each experimental group, aimed at assessing the consistency and reproducibility of the treatment effects. Power analysis was performed to confirm the statistical rigor of the experimental design. All data are reported as mean±standard error of the mean (SEM). Statistical analyses were conducted using Minitab software, and the generated plots were created using GraphPad Prism software. To determine significant differences related to various factors with a level of significance of α=0.05, ANOVA with Tukey's HSD (honestly significant difference) post hoc test was employed for multiple comparisons. The significance levels and corresponding p-values are indicated as follows: non-significant (ns), *p<0.05, **p<0.01, ***p<0.001, and ****p<0.0001.

### Synthesis and Characterization of GelMA

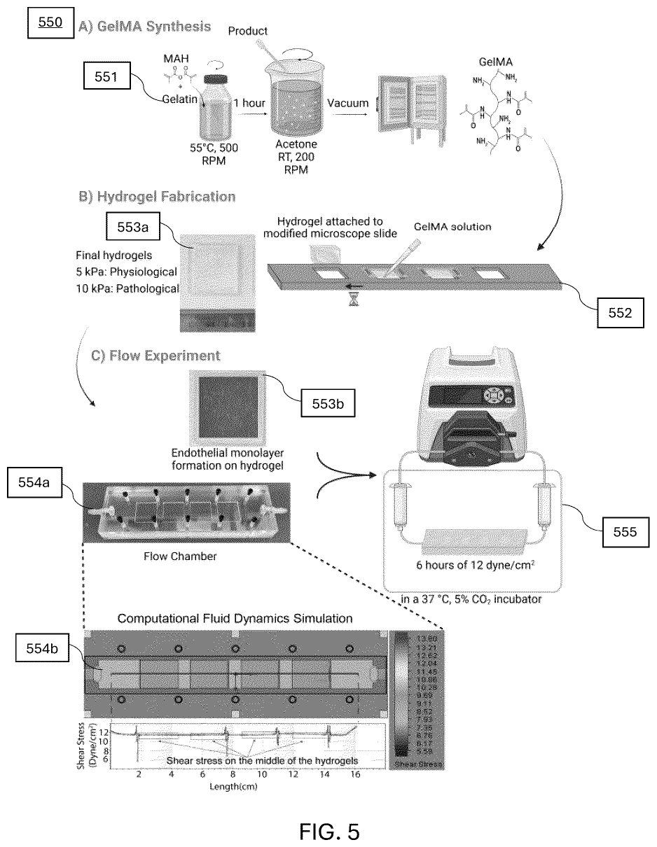

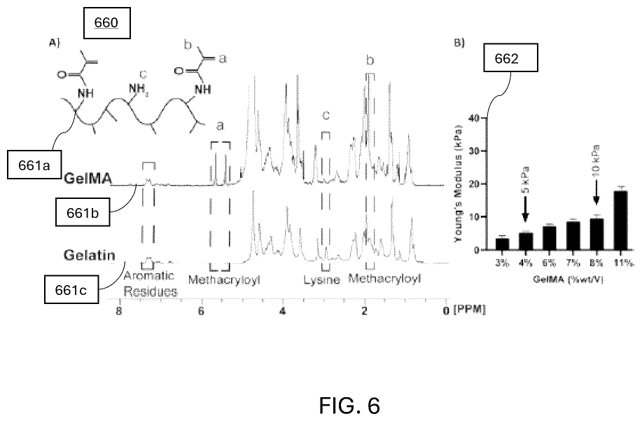

FIG. 5 illustrates the chemical synthesis of GelMA. GelMA synthesis 550 is shown by combining gelatin and MAH 551 to produce GelMA. 1H NMR spectra were utilized to determine the presence of Methacryloyl moieties following the chemical reactions. In FIG. 6, the appearance of new peaks 660 at 1.9, 5.4, and 5.6 ppm in GelMA 661b confirmed successful methacrylation 661a. Specifically, the chemical shifts around 5.4-5.6 ppm indicated the presence of vinylic protons, while the emergence of a peak at 1.9 ppm served as a marker for the methyl group (Kim et al., 2014; Rezaeeyazdi et al., 2018; Zhu et al., 2019). Furthermore, the absence of the peak at 3 ppm, compared to gelatin 661c, confirmed that the lysine residues were successfully substituted with methacrylate groups during the chemical reaction. The degree of methacrylation was estimated to be approximately 80%. NMR spectra also confirmed that the methacrylate groups were consumed during the polymerization process, as evidenced by the disappearance of the vinylic peaks (Rezaeeyazdi et al., 2018).

### Mechanical Properties of Hydrogels

The stiffness of GelMA hydrogels 662 was characterized via a compression test. As illustrated in FIG. 6, the compression moduli increased with the concentration of GelMA. Lower concentrations of GelMA resulted in the formation of softer hydrogels capable of withstanding large deformations, whereas higher polymer concentrations yielded harder hydrogels with increased stiffness. To mimic physiological and pathological substrate matrix stiffnesses, hydrogels with stiffnesses of 2.5, 5, and 10 kPa were selected for further experiments (Charbonier et al., 2019). These corresponded to GelMA concentrations of 3,4, and 8% (wt/vol), respectively. To investigate the gelation times, oscillatory time sweep experiments were conducted on GelMA hydrogels with concentrations of 3, 4, and 8% (wt/vol). Prior to the test, TEMED and APS were mixed with the polymer solutions, and the resulting mixture was loaded into the gap between the cone and plate. Initially, G′ values were smaller than G″ values, indicating that the crosslinks were insufficient to transform the liquid samples into a solid state. However, as the crosslinked network increased over time, the elastic behavior of the hydrogels became more prominent, and the G′ values surpassed the G″ values. The point at which the two moduli crossed over was designated as the gelation time, indicating the dominance of the elastic behavior in the gels. The G′ values exhibited an exponential increase and eventually reached a plateau, while G″ slightly decreased. As anticipated, the gelation process occurred faster for samples with higher polymer concentrations. The gelation times for the 3, 4, and 8% GelMA samples were approximately 132, 78, and 48 s, respectively. These times included the 30 s required for the pre-initiation process before initiating the test. Furthermore, the value of tan(8) decreased over time and was approximately 1 at the crossover point. As the concentration of GelMA increases, a decrease in gelation time was observed. This effect can be attributed to the reduced spatial distance between GelMA molecules, which increases the likelihood of collision for methacrylate groups. As a result, the crosslinking process occurs more rapidly, resulting in a shorter gelation time.

### Swelling Behavior of Hydrogels

The swelling behavior of hydrogels is an important property to consider in biomedical applications. Specifically, hydrogels used for in vitro studies of vascular ECs should exhibit minimal swelling to uphold a consistent flow regime and shear stress on the ECs on the gel are inserted into a flow chamber environment (this is beyond the scope of this paper and the focus of the ongoing work that will be reported in future publications). In this study, the influence of GelMA concentration and incubation time in PBS on the swelling ratio was investigate. An increase in GelMA concentration resulted in a decrease in the swelling ratio. This phenomenon can be attributed to the physical interlock and chemical binding of the polymer network, which impede the hydrogel's ability to swell. Meanwhile, the denominator of the Qm equation (Qm=hydrated weight/dry weight) increased due to a higher proportion of the solid phase remaining after lyophilization. The swelling ratios for the 2.5, 5, and 10 kPa hydrogels were measured as 41.7, 22.4, and 10.1, respectively. It is worth mentioning that the hydrogels, although they swell, eventually stabilize and reach an equilibrium point after 4 hours of incubation in PBS at 37° C. There was no significant difference between the samples at two different time points: the time of gel equilibration and day 4 after equilibration. Dimensional changes in the hydrogels by measuring the height alteration were also assessed. This was done by dividing the height of the hydrogel at equilibrium with the initial height of the sample immediately after polymerization. It was observed that there was no swelling and a non-significant shrinkage in the samples, which could be attributed to the highly cross-linked nature of the hydrogels.

### The Effect of Substrate Composition on GCX

GCX sensitivity to the substrate was first assessed by examining the influence of three substrate materials, fibronectin, gelatin, and unpolymerized GelMA, which were initially used as coatings on glass slides to eliminate the potential mechanical influence that hydrogel would have on cell behavior. Fibronectin coating was included in this work because it is commonly employed as a coating component in EC studies for atherosclerosis. However, its deposition occurs during the diseased phase when ECs gain an inflammatory phenotype (Hahn et al., 2009; Hamrangsekachaee et al., 2022) and, therefore, the use of fibronectin may interfere with the natural behavior of cells in response to their environment. Gelatin and GelMA coatings were both used because they are derived from collagen, which is physiologically relevant. GelMA is considered to be the most ideal coating because it can be extended from a coating format to a gel format easily and relatively inexpensively, and in a manner that will permit substrate cell and bioactive molecule encapsulation. GelMA can also be tuned to increase the concentration of adhesive RGD molecules, and better support cell adhesion and growth. It was unclear whether and how the coatings made of fibronectin, gelatin, and GelMA, of variable concentrations, would affect the expression of GCX.

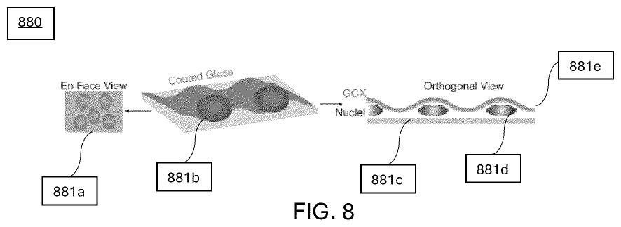

To address this question, whole GCX (WGA) was examined, and the normalized GCX expression (MFI) from the en face view 881a of microscopic images 880 was used to evaluate the effect of substrate material 881c on apical GCX expression (FIG. 8) of the cells 881d with the cells and GCX on the coated glass 881b. The orthogonal views of z-stack images were used to measure the thickness of the GCX 881e. The results showed that the substrate material had a significant effect on whole GCX (WGA) expression. Specifically, gelatin significantly increased expression by more than 30%, while 1× GelMA significantly decreased expression by about 30%, compared to fibronectin. While overall expression responded to differences in substrate composition (fibronectin versus gelatin versus 1×GelMA), the thickness of the whole GCX (WGA) did not change substantially in response to substrate composition although there was a trend indicating that the measured thickness increased when GCX expression decreased. For GelMA, GCX expression was further assessed by conducting a dose-response study of the effect of increasing GelMA concentration while controlling for stiffness. The concentration of GelMA was increased 5, 10, 100, and 400 times to assess changes in GCX expression. In a control experiment, the same was done with gelatin. The results showed that in GelMA samples, the normalized expression (MFI) of the whole GCX (WGA) nearly doubled and then reached a plateau in response to a 5- and 10-fold increase in GelMA concentration. Higher concentrations (100× and 400×) of GelMA coating led to cell detachment presumably due to the high concentration of methacrylate groups, potentially causing cell toxicity and detachment. In the control experiments with gelatin, increasing the concentration of gelatin did not significantly affect the expression of the whole GCX (WGA), as both the normalized expression (MFI) and thickness of the GCX remained similar across the samples. The results suggest that the substrate material can indeed impact GCX expression. The use of gelatin coating and GelMA coating at moderately high concentrations may be better than fibronectin to accurately elicit natural GCX-mediated behavior of ECs for atherosclerosis-related studies.

### The Effect of Substrate Stiffness on GCX

To investigate the impact of substrate stiffness on GCX expression, hydrogels with stiffness values of 2.5, 5 and 10 kPa were employed. The hydrogels with stiffness values of 2.5 and 5 kPa hydrogels represented matrices with physiological stiffness, while the 10 kPa hydrogels represented pathological stiffness (Mahmoud et al., 2021b). Previous studies have shown that stiffness can disrupt the expression of glypican 1, a core protein of the GCX, as well as HS, a polysaccharide component of GCX, on fibronectin-coated polyacrylamide hydrogels (Mahmoud et al., 2021a). Example 1 presents a continued study of GCX mechano-response to substrate stiffness and sought to do so by using non-swelling GelMA hydrogels in preparation for future research that will translate the GelMA platform to a flow chamber with geometry and flow parameter constraints.

When cells were plated directly on the GelMA hydrogels as initially planned, it was found that cells did not consistently adhere well and exhibited delayed proliferation. Challenges were found to be particularly relevant to 2.5 kPa hydrogels. To overcome the potential adverse impact of GelMA on cellular adhesion and proliferation-to-confluence, the hydrogels were coated with 1× gelatin prior to cell seeding. To confirm that the gelatin could not change the GelMA mechanical properties detected by ECs, the gelatin was FITC-conjugated to visualize the gelatin coat thickness. After washing the hydrogel samples, it could be seen via FTIC that the gelatin is very thin on both soft and stiff hydrogels and, therefore, unable to affect the mechanical properties of GelMA. In addition, on 2.5 kPa hydrogels, cell attachment was monitored, and initial attachment to the gelatin-coated substrates was found to be superior compared to non-coated substrates. Cells exhibited sufficient attachment to 10 kPa hydrogels irrespective of coating (data not reported). Subsequently, the influence of uncoated GelMA versus gelatin-coated GelMA on GCX expression was investigated using WGA labeling, and no significant differences were observed between the coated and non-coated hydrogels. Therefore, the application of gelatin coating on GelMA hydrogel yielded fine-tuned cell culture optimization: gelatin coating of GelMA solely supported cell attachment to the softer gels and did not impact attachment on stiffer gels or GCX expression on hydrogels with different stiffnesses.

For cells grown on gelatin-coated GelMA hydrogels, the effects of stiffnesses of 2.5 kPa, 5 kPa, versus 10 kPa were compared to each other. Qualitatively, WGA-labeled GCX was abundantly expressed. In en face images, occasional aggregation of the WGA lectin was seen. When scanning the en face images, the aggregates could be thought to be marking cell junctions. However, when scanning the cross-sectional images, the aggregates appeared to be randomly distributed and less obvious. Quantitative analysis of the en face images revealed that there were no significant differences in GCX (WGA) expression (MFI) detected when comparing GCX expression in 2.5 kPa, 5 kPa, versus 10 kPa conditions. Similarly, the thickness measurements did not reveal any significant differences across samples with different stiffness values. These observations prompted us to investigate the prevailing saccharide components of the GCX. The ECs displayed lower HS expression on 10 kPa hydrogels, which resembled pathological stiffness, while no significant difference was observed between 2.5 and 5 kPa hydrogels, representing the physiological stiffness of blood vessels. Similarly, the percentage of the EC covered area occupied by HS decreased from 74% (2.5 kPa) and 69% (5 kPa) to 56% (10 kPa) when transitioning from physiological to pathological matrix stiffness. Likewise, the thickness of the HS was decreased by increasing stiffness from physiological to pathological substrates. These findings align with the data published by Mahmoud et al. (Mahmoud et al., 2021b), which demonstrated HS downregulation with increasing stiffness. It is worth noting that in some areas, the orthogonal view revealed intracellular signals, despite the fact that the HS staining procedure did not include cell membrane permeabilization of the samples. Perhaps the long period of primary antibody incubation allowed the antibody ample time to passively diffuse through the cell membrane to the cytoplasm.

Next, the presence of SA was examined. Qualitatively, it could be observed in en face views that there was an apparent pattern of SA expression at cell junctions. However, the cross-sectional views did not provide further insight into any tendency for SA to mark cell junctions. Therefore, conclusions about cell junctional SA expression are to be approached with caution. The cross-sectional views pointed to something else that was of interest. The images showed occasionally that SA was present not only on the apical membrane of the cultured cells but also on the basal membrane. There was an attempt to quantify differential SA expression at the apical surface versus at the basal surface. It appeared to be predominantly basal at 2.5 kPa and switch to predominantly apical at 10 kPa. However, these are cautious conclusions because the statistical analysis of this phenomenon could not be performed. Expression of basal GCX was rare and left a small sample size that could not be adequately processed with statistical tests. When examining the quantitative results, a correlation between substrate stiffness and SA expression was revealed, with cells exhibiting higher SA expression on stiffer substrates. Compared to 2.5 kPa hydrogels, normalized SA expression (MFI) increased by 2.5 times on 5 kPa hydrogels and 3.8 times on 10 kPa hydrogels. However, there were no significant differences in area coverage or thickness of SA between the substrates with different stiffness levels. Although the thickness tended to decrease with increasing stiffness, the changes were not statistically significant.

The last component studied was HA. Qualitatively, the cells appeared to express less HA than HS and SA. The was no apparent HA preference for cell junctions or basal expression of HA, which were interesting observations that were made for HS and SA. Quantitatively, normalized HA expression increased by a statistically significant 1.48-fold from 2.5 kPa to 5 kPa conditions but decreased back to approximately the same level as 2.5 kPa when stiffness increased from 5 to 10 kPa. There was no significant difference when comparing the effects of 2.5 kPa to 10 kPa. The percentage area of ECs covered with HA did not show a significant difference among the samples in different stiffness conditions, although a decreasing trend was observed on stiffer substrates. Lastly, the thickness of HA layer exhibited a statistically significant decrease for samples exposed to highest stiffness conditions compared to samples exposed to lowest stiffness conditions.

### Discussion

The GCX is a crucial element of atherosclerosis, but its delicate and complex structure has made it difficult to study using traditional methods until recent technological advancements, which have made it more feasible (Hamrangsekachaee et al., 2022). Additionally, relatively recent studies have demonstrated the direct response of GCX to apical flow-derived shear stress, while many studies have overlooked the impact of the basal substrate on GCX features and function. To improve the accuracy of in vitro studies, it is essential to closely mimic the in vivo microenvironment, which includes a variety of mechanical, electrical, chemical, and other cues. As a first step toward improving model accuracy, Example 1 used a non-swelling hydrogel-based substrate with tunable properties. This substrate serves as a more physiologically relevant platform for incorporating mechanical stiffness of both healthy and diseased subendothelial matrices into the models. The results of this study will influence the substrate material choice for future studies. Furthermore, GCX properties have been expanded upon recently published findings on the effects of substrate matrix mechanical properties on GCX (Mahmoud et al., 2021b), adopting a holistic approach that provides insights into the responses of the entire GCX, as well as its subcomponent and the ECs, to different solid-derived forces. In future work, the substrate matrix model will be combined with different types of flow conditions to create a further optimized in vitro model for studying EC behavior and functionality.

Development of a Hydrogel-Based Substrate with Tunable Properties to Mimic Healthy and Diseased Subendothelial Matrix

To improve upon existing models for studying EC and GCX in the context of atherosclerosis, this work has developed a biologically, chemically, and mechanically relevant EC substrate. Several candidate substrate biomaterials such as polyethylene glycol, and tropoelastin were pilot tested, but it was determined that a non-swelling gelatin-based option is most suitable for the long-term goal of developing an optimized in vitro model introducing flow conditions for studying EC behavior. It is acknowledged that gelatin-based substrates have been extensively studied in recent decades, and their advantages in terms of sustainability, cost effectiveness, and biocompatibility have been established (Gómez-Guillén et al., 2011; Lee et al., 2015; Shirahama et al., 2016; Zhu et al., 2019). However, there is a unique aspect to this approach, as two well-established protocols were combined to enhance the reproducibility of the substrate batches while keeping the process simple (Kim et al., 2014; Zhu et al., 2019). Additionally, by modifying gelatin into GelMA, this study successfully achieved the goal of creating substrates with different stiffness levels while maintaining the shape and dimension of the substrate. This will be advantageous when integrating the substrate into a flow channel setting in the future, as it will prevent any unexpected flow disturbance resulting from hydrogel swelling.

Regulation of Substrate Components and their Concentration

Since the mechanical properties of the hydrogel substrate are directly influenced by the concentration of GelMA, it was important to assess GCX expression based on both the composition publications due to its high cell attachment properties (Stroka and Aranda-Espinoza, 2011; Yeh et al., 2012; Mahmoud et al., 2021a; Mahmoud et al., 2021b). Surprisingly, the overall expression of GCX on gelatin and GelMA was significantly different compared to fibronectin. Gelatin increased GCX expression, while GelMA initially decreased GCX expression. However, as the concentration of GelMA was increased, there was a significant increase in GCX expression, approaching the level of GCX expression on gelatin coated glasses. Interestingly, GCX expression did not change significantly in response to coating with higher concentrations of gelatin. These observations suggest that under static conditions with the same substrate stiffness, GCX expression can be upregulated to a certain extent. The concentration of GelMA determines the stiffness of hydrogels, but it can be concluded that the number of cell-adhesive sequences in GelMA hydrogels are significantly above the threshold. These findings also suggest that gelatin or GelMA (5× and 10×) could be considered as substitutes for fibronectin coating in atherosclerosis-related research using coated glass substrates, especially since it is known that fibronectin becomes a predominant constituent of the substrate matrix that ECs reside on under disease conditions. Elevated fibronectin levels have been shown to have adverse effects on the deposition, organization, and stability of other matrix adhesion proteins, as well as functional events such as endothelial permeability, cell adhesion, and cell proliferation (Sottile and Hocking, 2002; Chiang et al., 2009). Numerous studies have also demonstrated a correlation between high fibronectin levels and increased risk of atherosclerosis in human patients (Magnusson and Mosher, 1998; Tzanatos et al., 2009; Holm Nielsen et al., 2020). Therefore, it is important to limit or completely avoid experimental models that incorporate substrate protein that exacerbates atherosclerosis, as they may interfere with cell behavior and subsequently affect research data.

Increasing Substrate Stiffness Differentially Dysregulates Individual Polysaccharides without Altering Overall GCX

Numerous studies have demonstrated the impact of substrate stiffness on the behavior and function of ECs, including proliferation, migration, and even ECs stiffness. (Byfield et al., 2009; Lampi et al., 2017; Zhong et al., 2018; Bastounis et al., 2019; Kruger-Genge et al., 2021). However, only a few studies have specifically examined the influence of stiffness on GCX expression (Mahmoud et al., 2021a; Mahmoud et al., 2021b), and Example 1 aimed to contribute to this area of research. To understand the effect of substrate stiffness on GCX integrity, a holistic approach was implemented, treating the GCX as a homogeneous saccharide coating that covers the cell surface and labeling it with WGA. The results indicated that the intensity and thickness of expressed whole GCX did not change in response to substrate stiffness. This unexpected finding contradicted the initial hypothesis and differed from previous reports (Mahmoud et al., 2021a; Mahmoud et al., 2021b) that suggested a correlation between stiffness and GCX expression. Consequently, this shifted focus to examining the alterations in three major polysaccharide components of the GCX.

Example 1 initially examined the presence of HS. In healthy ECs, HS is continuously produced in the endoplasmic Golgi apparatus. It is the most predominant component among GAGs, comprising 60%-90% of GCX GAGs, and plays a crucial role in various functions. The physiological function of this GAG can be influenced by more than 4,000 different possible sulfation patterns (Reitsma et al., 2007; Spiess, 2017). These findings indicate that HS is highly sensitive to substrate stiffness, which aligns with previous research (Mahmoud et al., 2021a; Mahmoud et al., 2021b). The expression (MFI; aggregate density) and thickness of HS decreased on the pathologically stiff surface (10 kPa), consistent with previous studies (Mahmoud et al., 2021a). The decrease in HS has significant adverse implications, as it is well-established that HS serves as a mechanotransducing factor involved in shear-induced NO production, cell motility, cell proliferation, and cell remodeling (Florian et al., 2003; Pahakis et al., 2007; Yao et al., 2007; Ebong et al., 2014). Although the present study did not involve flow-derived shear stress, these results suggested that a stiffer basement membrane could potentially interfere with flow-dependent cell functions, such as cell alignment and NO production, due to its associated lowering of HS expression. Additionally, HS acts as a mask for adhesion molecules like intercellular adhesion molecule 1 (ICAM-1) and vascular adhesion molecule 1 (VCAM-1) (Mulivor and Lipowsky, 2002). The decrease in the distribution and thickness of HS, as observed with increasing stiffness, implies that a stiff substrate may increase the likelihood of inflammatory cell adhesion via exposing ICAM-1 and VCAM-1 molecules on the EC surface. This conclusion is supported by previous reports showing that stiffer hydrogels promoted immune cell trans-endothelial migration (Huynh et al., 2011), and one possible reason for this observation could be the downregulation of HS on stiffer hydrogels. However, it is worth noting that while these findings regarding HS results and their implications align with prior published work, the disparity between the presence of HS and the overall GCX expression as measured by WGA was an unexpected finding.

The results obtained from examining SA provide an explanation for the disparity between the overall GCX and HS. Although the thickness and coverage area of SA did not change significantly, its expression (MFI; aggregate density) was substantially upregulated on stiffer substrates. This clarifies the discrepancy between HS and the overall GCX, providing valuable insights and implications. One implication of increased SA, due to its repulsive negative charge (Wallach and Kamat, 1966), is that it enhances the maintenance of the GCX's role as a permeability barrier. Another significant implication of increased SA, as it contains binding sites for recruiting immune cells and pathogens (Schauer, 2009; Chang and Nizet, 2014; Macauley et al., 2014; Mahajan and Pillai, 2016; Pearce and Laubli, 2016), is that it leads to enhanced inflammation. Considering these implications in conjunction with the HS results, it suggests that while the overall structure of the whole GCX (as indicated by WGA) is maintained, the relative concentrations of its individual components are altered. Furthermore, ECs respond to increasing stiffness conditions by adjusting the presence of HS and SA, not simply to counterbalance each other, but potentially to cooperate in adversely impacting EC functionality. In summary, the decrease in HS impairs mechanotransduction and exposes adhesion molecules, while the increase in SA recruits immune cells and pathogens, contributing to altered EC functionality under stiffer conditions.

To explore more about the effect of stiffness on the specific component of GCX, the presence of HA was examined. HA is a non-sulfated GAG that is part of the GCX and is bound to it through CD44 receptors on ECs. When HA is shed, it contributes to the loss of barrier functionality and can be associated with various disease conditions (Vlahu et al., 2012; Padberg et al., 2014; Tarbell et al., 2014). Previous studies have shown that the suppression of HA synthesis in mouse models leads to increased leukocyte adhesion and accelerated atherosclerosis (Nagy et al., 2010). Based on this well-established information about HA, it was expected to observe a less distributed HA with lower thickness in EC cultures on pathologically stiff substrates. Although it was observed to decrease in thickness, it was difficult to interpret due to the lack of meaningful correlations between the change of HA expression (measured by MFI; aggregate density) and the change in HA coverage of ECs. These results suggest that stiffness has a negligible impact on promoting HA production. Future work plans to investigate whether the regulation of HA synthesis by stiffness can be induced by incorporating fluid flow as an additional factor in this model. By stimulating ECs with combined fluid flow and substrate stiffness forces, a more complex but closer to accurate in vitro model is created, which would provide further insights into HA dynamics.

In some embodiments, this study demonstrates certain limitations that will be addressed in subsequent stages of the project. Firstly, the primary focus of the study centered on investigating the saccharide chains of the GCX in the absence of shear stress conditions. Shear stress is known to exert a critical influence on altering GCX. Consequently, the study did not extensively explore other components, including core proteins and the underlying mechanisms of GCX dysregulation. To establish a more comprehensive understanding of GCX, the forthcoming stage of this research will implement shear stress within the experimental setup. By doing so, the study will attain a more accurate representation of GCX responses to mechanical forces. The HUVECs used in this investigation do not fully represent the native endothelial cells of human arteries. However, their selection was based on a substantial body of data that allowed for the collection of comparable results and insights. In addition, the study did not include vascular smooth muscle cells (VSMCs), which are known to play a prominent role in atherosclerosis. The upcoming stages of the study will incorporate VSMCs to develop a more comprehensive multi-cell system that can be subjected to shear stress, thereby enhancing the accuracy of in vivo emulation. It is important to note that the hydrogels used in this study exhibited non-swelling behavior and possessed the capability to embed cells. Thus, the model can be exposed to shear stress, facilitating a more accurate representation of in vivo conditions.

### Conclusion

In this work, Example 1 successfully modified gelatin through a straightforward method using methacrylate anhydrate, resulting in a reproducible biomaterial source for a substrate supporting the culture of ECs. Subsequent chemical, physical, and mechanical characterization confirmed the tunable nature of the hydrogel substrate. Notably, for the first time, gelatin and GelMA were compared to fibronectin, and it was found that gelatin at the same concentration of fibronectin could enhance GCX expression, while unpolymerized GelMA at higher concentrations also showed improved GCX expression. This finding provides a more cost-effective and sustainable alternative to fibronectin for endothelial studies. Furthermore, the impact of substrate chemistry and stiffness on GCX expression was evaluated. Interestingly, whole GCX expression was unchanged when ECs were cultured on physiologically soft versus pathologically stiff hydrogels. However, the individual GCX polysaccharides showed dysregulation in response to the mechanics of the hydrogels, with HS being downregulated and SA upregulated with increasing substrate stiffness. There was no strong correlation found between HA dysregulation and stiffness. The different responses of the various GCX components likely have direct or indirect effects on EC functions, given the diverse role of each component. It is important to note that the study did not assess EC functions beyond GCX production, which is the subject of a separate ongoing investigation where the EC substrate presented in this study is combined with fluid flow to create a robust in vitro model. The findings of this study provide new insights into how ECs respond to external forces and adapt their GCX to regulate EC function under various mechanical conditions.

### REFERENCES

Bastounis, E. E., Yeh, Y.-T., and Theriot, J. A. (2019). Subendothelial stiffness alters endothelial cell traction force generation while exerting a minimal effect on the transcriptome. Sci. Rep. 9, 18209. doi:10.1038/s41598-019-54336-2
Benetos, A., Waeber, B., Izzo, J., Mitchell, G., Resnick, L., Asmar, R., et al. (2002). Influence of age, risk factors, and cardiovascular and renal disease on arterial stiffness: clinical applications. Am. J. Hypertens. 15, 1101-1108. doi:10.1016/s0895-7061(02)03029-7
Bonetti, P. O., Lerman, L. O., and Lerman, A. (2003). Endothelial dysfunction. Arteriosclerosis, Thrombosis, Vasc. Biol. 23, 168-175. doi:10.1161/01.atv.0000051384.43104.fc
Byfield, F. J., Reen, R. K., Shentu, T.-P., Levitan, I., and Gooch, K. J. (2009). Endothelial actin and cell stiffness is modulated by substrate stiffness in 2D and 3D. J. Biomechanics 42, 1114-1119. doi:10.1016/j.jbiomech.2009.02.012
Chang, Y.-C., and Nizet, V. (2014). The interplay between Siglecs and sialylated pathogens. Glycobiology 24, 818-825. doi:10.1093/glycob/cwu067
Charbonier, F. W., Zamani, M., and Huang, N. F. (2019). Endothelial cell mechanotransduction in the dynamic vascular environment. Adv. Biosyst. 3, 1800252. doi:10.1002/adbi.201800252
Chiang, H. Y., Korshunov, V. A., Serour, A., Shi, F., and Sottile, J. (2009). Fibronectin is an important regulator of flow-induced vascular remodeling. Arterioscler. Thromb. Vasc. Biol. 29, 1074-1079. doi:10.1161/atvbaha.108.181081
Chirinos, J. A. (2012). Arterial stiffness: basic concepts and measurement techniques. J. Cardiovasc. Transl. Res. 5, 243-255. doi:10.1007/s12265-012-9359-6
Cuvellier, M., Ezan, F., Oliveira, H., Rose, S., Fricain, J.-C., Langouët, S., et al. (2021). 3D culture of HepaRG cells in GelMa and its application to bioprinting of a multicellular hepatic model. Biomaterials 269, 120611. doi:10.1016/j.biomaterials.2020.120611
David, G., Bai, X. M., Van Der Schueren, B., Cassiman, J. J., and Van Den Berghe, H. (1992). Developmental changes in heparan sulfate expression: in situ detection with mAbs. J. Cell Biol. 119, 961-975. doi:10.1083/jcb.119.4.961
Deanfield, J. E., Halcox, J. P., and Rabelink, T. J. (2007). Endothelial function and dysfunction. Circulation 115, 1285-1295. doi:10.1161/circulationaha.106.652859
Ebong, E. E., Lopez-Quintero, S. V., Rizzo, V., Spray, D. C., and Tarbell, J. M. (2014). Shear-induced endothelial NOS activation and remodeling via heparan sulfate, glypican-1, and syndecan-1. Integr. Biol. 6, 338-347. doi:10.1039/c3ib40199e
Ebong, E. E., Macaluso, F. P., Spray, D. C., and Tarbell, J. M. (2011). Imaging the endothelial glycocalyx in vitro by rapid freezing/freeze substitution transmission electron microscopy. Arterioscler. Thromb. Vasc. Biol. 31, 1908-1915. doi:10.1161/atvbaha.111.225268
Ebong, E. E., Spray, D. C., and Tarbell, J. M. (2010). “The endothelial glycocalyx: its structure and role in eNOS mechano-activation,” in Proceedings of the 2010 IEEE 36^{th} Annual Northeast Bioengineering Conference (NEBEC), New York, NY, USA, 26-28 Mar. 2010, 1-2.
Engler, A. J., Richert, L., Wong, J. Y., Picart, C., and Discher, D. E. (2004). Surface probe measurements of the elasticity of sectioned tissue, thin gels and polyelectrolyte multilayer films: correlations between substrate stiffness and cell adhesion. Surf. Sci. 570, 142-154. doi:10.1016/j.susc.2004.06.179
Florian, J. A., Kosky, J. R., Ainslie, K., Pang, Z., Dull, R. O., and Tarbell, J. M. (2003). Heparan sulfate proteoglycan is a mechanosensor on endothelial cells. Circ. Res. 93, e136-e142. doi:10.1161/01.res.0000101744.47866.d5
Fuhrmann, I. K., Steinhagen, J., Rüther, W., and Schumacher, U. (2015). Comparative immunohistochemical evaluation of the zonal distribution of extracellular matrix and inflammation markers in human meniscus in osteoarthritis and rheumatoid arthritis. Acta Histochem. 117, 243-254. doi:10.1016/j.acthis.2014.12.009
Gabor, F., Wirth, M., Jurkovich, B., Haberl, I., Theyer, G., Walcher, G., et al. (1997). Lectin-mediated bioadhesion: proteolytic stability and binding-characteristics of wheat germ agglutinin and Solanum tuberosum lectin on Caco-2, HT-29 and human colonocytes. J. Control. Release 49, 27-37. doi:10.1016/s0168-3659(97)00057-6
Gómez-Guillén, M. C., Giménez, B., López-Caballero, M. E., and Montero, M. P. (2011). Functional and bioactive properties of collagen and gelatin from alternative sources: a review. Food Hydrocoll. 25, 1813-1827. doi:10.1016/j.foodhyd.2011.02.007
Hahn, C., Orr, A. W., Sanders, J. M., Jhaveri, K. A., and Schwartz, M. A. (2009). The subendothelial extracellular matrix modulates JNK activation by flow. Circulation Res. 104, 995-1003. doi:10.1161/circresaha.108.186486
Hamrangsekachaee, M., Wen, K., Bencherif, S. A., and Ebong, E. E. (2022). Atherosclerosis and endothelial mechanotransduction: current knowledge and models for future research. Am. J. Physiology-Cell Physiology 324, C488-C504. doi:10.1152/ajpcell.00449.2022
Harding, I. C., Mitra, R., Mensah, S. A., Herman, I. M., and Ebong, E. E. (2018). Proatherosclerotic disturbed flow disrupts caveolin-1 expression, localization, and function via glycocalyx degradation. J. Transl. Med. 16, 364. doi:10.1186/s12967-018-1721-2
Heinegird, D., and Hascall, V. C. (1974). Aggregation of cartilage proteoglycans. J. Biol. Chem. 249, 4250-4256. doi:10.1016/s0021-9258(19)42509-x
Holm Nielsen, S., Jonasson, L., Kalogeropoulos, K., Karsdal, M. A., Reese-Petersen, A. L., Auf DemKeller, U., et al. (2020). Exploring the role of extracellular matrix proteins to develop biomarkers of plaque vulnerability and outcome. J. Intern. Med. 287, 493-513. doi:10.1111/joim.13034
Huynh, J., Nishimura, N., Rana, K., Peloquin, J. M., Califano, J. P., Montague, C. R., et al. (2011). Age-related intimal stiffening enhances endothelial permeability and leukocyte transmigration. Sci. Transl. Med. 3, 112ra122. doi:10.1126/scitranslmed3002761
Ishigaki, H., and Itoh, Y. (2022). “Detection of sialic acids on the cell surface using flow cytometry,” in Glycovirology: methods and protocols. Editor Y. SUZUKI (New York, NY: Springer US).
Jansen, K. A., Atherton, P., and Ballestrem, C. (2017). Mechanotransduction at the cell-matrix interface. Seminars Cell and Dev. Biol. 71, 75-83. doi:10.1016/j.semcdb.2017.07.027
Kim, J., Bencherif, S. A., Li, W. A., and Mooney, D. J. (2014). Cell-friendly inverse opal-like hydrogels for a spatially separated Co-culture system. Macromol. Rapid Commun. 35, 1578-1586. doi:10.1002/marc.201400278
Klein, E. A., Yin, L., Kothapalli, D., Castagnino, P., Byfield, F. J., Xu, T., et al. (2009). Cell-cycle control by physiological matrix elasticity and in vivo tissue stiffening. Curr. Biol. 19, 1511-1518. doi:10.1016/j.cub.2009.07.069
Kohn, J. C., Lampi, M. C., and Reinhart-King, C. A. (2015). Age-related vascular stiffening: causes and consequences. Front. Genet. 6, 112. doi:10.3389/fgene.2015.00112
Krishnamoorthy, S., Noorani, B., and Xu, C. (2019). Effects of encapsulated cells on the physical-mechanical properties and microstructure of gelatin methacrylate hydrogels. Int. J. Mol. Sci. [Online]20, 5061. doi:10.3390/ijms20205061
Krüger-Genge, A., Hauser, S., Neffe, A. T., Liu, Y., Lendlein, A., Pietzsch, J., et al. (2021). Response of endothelial cells to gelatin-based hydrogels. ACS Biomaterials Sci. Eng. 7, 527-540. doi:10.1021/acsbiomaterials.0c01432
Lampi, M. C., Guvendiren, M., Burdick, J. A., and Reinhart-King, C. A. (2017). Photopatterned hydrogels to investigate the endothelial cell response to matrix stiffness heterogeneity. ACS Biomaterials Sci. Eng. 3, 3007-3016. doi:10.1021/acsbiomaterials.6b00633
Laroia, S. T., Ganti, A. K., Laroia, A. T., and Tendulkar, K. K. (2003). Endothelium and the lipid metabolism: the current understanding. Int. J. Cardiol. 88, 1-9. doi:10.1016/s0167-5273(02)00366-2
Lavrentieva, A., Fleischhammer, T., Enders, A., Pirmahboub, H., Bahnemann, J., and Pepelanova, I. (2020). Fabrication of stiffness gradients of GelMA hydrogels using a 3D printed micromixer. Macromol. Biosci. 20, 2000107. doi:10.1002/mabi.202000107
Lee, B. H., Shirahama, H., Cho, N.-J., and Tan, L. P. (2015). Efficient and controllable synthesis of highly substituted gelatin methacrylamide for mechanically stiff hydrogels. RSC Adv. 5, 106094-106097. doi:10.1039/c5ra22028a
Lee, Y., Kischuk, E., Crist, S., Ratliff, T. L., and Thompson, D. H. (2017). Targeting and internalization of liposomes by bladder tumor cells using a fibronectin attachment protein-derived peptide-lipopolymer conjugate. Bioconjugate Chem. 28, 1481-1490. doi:10.1021/acs.bioconjchem.7b00153
Macauley, M. S., Crocker, P. R., and Paulson, J. C. (2014). Siglec-mediated regulation of immune cell function in disease. Nat. Rev. Immunol. 14, 653-666. doi:10.1038/nri3737
Magnusson, M. K., and Mosher, D. F. (1998). Fibronectin. Arteriosclerosis, Thrombosis, Vasc. Biol. 18, 1363-1370. doi:10.1161/01.atv.18.9.1363
Mahajan, V. S., and Pillai, S. (2016). Sialic acids and autoimmune disease. Immunol. Rev. 269, 145-161. doi:10.1111/imr.12344
Mahmoud, M., Cancel, L., and Tarbell, J. M. (2021a). Matrix stiffness affects glycocalyx expression in cultured endothelial cells. Front. Cell Dev. Biol. 9, 731666. doi:10.3389/fcell.2021.731666
Mahmoud, M., Mayer, M., Cancel, L. M., Bartosch, A. M., Mathews, R., and Tarbell, J. M. (2021b). The glycocalyx core protein Glypican 1 protects vessel wall endothelial cells from stiffness-mediated dysfunction and disease. Cardiovasc Res. 117, 1592-1605. doi:10.1093/cvr/cvaa201
Mensah, S. A., Cheng, M. J., Homayoni, H., Plouffe, B. D., Coury, A. J., and Ebong, E. E. (2017). Regeneration of glycocalyx by heparan sulfate and sphingosine 1-phosphate restores inter-endothelial communication. PLoS One 12, e0186116. doi:10.1371/journal.pone.0186116
Mensah, S. A., Nersesyan, A. A., Harding, I. C., Lee, C. I., Tan, X., Banerjee, S., et al. (2020). Flow-regulated endothelial glycocalyx determines metastatic cancer cell activity. FASEB J. 34, 6166-6184. doi:10.1096/fj.201901920r
Mitchell, G. F., Hwang, S.-J., Vasan, R. S., Larson, M. G., Pencina, M. J., Hamburg, N. M., et al. (2010). Arterial stiffness and cardiovascular events. Fram. Heart Study 121, 505-511. doi:10.1161/circulationaha.109.886655
Mulivor, A. W., and Lipowsky, H. H. (2002). Role of glycocalyx in leukocyteendothelial cell adhesion. Am. J. Physiology-Heart Circulatory Physiology 283, H1282-H1291. doi:10.1152/ajpheart.00117.2002
Nagy, N., Freudenberger, T., Melchior-Becker, A., Röck, K., Ter Braak, M., Jastrow, H., et al. (2010). Inhibition of hyaluronan synthesis accelerates murine atherosclerosis. Circulation 122, 2313-2322. doi:10.1161/circulationaha.110.972653
Newby, A. C. (2008). Metalloproteinase expression in monocytes and macrophages and its relationship to atherosclerotic plaque instability. Arteriosclerosis, Thrombosis, Vasc. Biol. 28, 2108-2114. doi:10.1161/atvbaha.108.173898
Orr, A. W., Sanders, J. M., Bevard, M., Coleman, E., Sarembock, I. J., and Schwartz, M. A. (2005). The subendothelial extracellular matrix modulates NF-kappaB activation by flow: a potential role in atherosclerosis. J. Cell Biol. 169, 191-202. doi:10.1083/jcb.200410073
Padberg, J.-S., Wiesinger, A., Di Marco, G. S., Reuter, S., Grabner, A., Kentrup, D., et al. (2014). Damage of the endothelial glycocalyx in chronic kidney disease. Atherosclerosis 234, 335-343. doi:10.1016/j.atherosclerosis.2014.03.016
Pahakis, M. Y., Kosky, J. R., Dull, R. O., and Tarbell, J. M. (2007). The role of endothelial glycocalyx components in mechanotransduction of fluid shear stress. Biochem. Biophys. Res. Commun. 355, 228-233. doi:10.1016/j.bbrc.2007.01.137
Pearce, O. M., and Laubli, H. (2016). Sialic acids in cancer biology and immunity. Glycobiology 26, 111-128. doi:10.1093/glycob/cwv097
Peloquin, J., Huynh, J., Williams, R. M., and Reinhart-King, C. A. (2011). Indentation measurements of the subendothelial matrix in bovine carotid arteries. J. biomechanics 44, 815-821. doi:10.1016/j.jbiomech.2010.12.018
Reitsma, S., Slaaf, D. W., Vink, H., Van Zandvoort, M. A. M. J., and Oude Egbrink, M. G. A. (2007). The endothelial glycocalyx: composition, functions, and visualization. Pflugers Archiv—Eur. J. Physiology 454, 345-359. doi:10.1007/s00424-007-0212-8
Rezaeeyazdi, M., Colombani, T., Memic, A., and Bencherif, S. A. (2018). Injectable hyaluronic acid-co-gelatin cryogels for tissue-engineering applications. Mater. [Online]11, 1374. doi:10.3390/ma11081374
Schauer, R. (2009). Sialic acids as regulators of molecular and cellular interactions. Curr. Opin. Struct. Biol. 19, 507-514. doi:10.1016/j.sbi.2009.06.003
Shirahama, H., Lee, B. H., Tan, L. P., and Cho, N.-J. (2016). Precise tuning of facile one-pot gelatin methacryloyl (GelMA) synthesis. Sci. Rep. 6, 31036. doi:10.1038/srep31036
Sottile, J., and Hocking, D. C. (2002). Fibronectin polymerization regulates the composition and stability of extracellular matrix fibrils and cell-matrix adhesions. Mol. Biol. Cell 13, 3546-3559. doi:10.1091/mbc.e02-01-0048
Spiess, B. D. (2017). Heparin: effects upon the glycocalyx and endothelial cells. J. Extra Corpor. Technol. 49, 192-197. doi:10.1051/ject/201749192
Stroka, K. M., and Aranda-Espinoza, H. (2011). Endothelial cell substrate stiffness influences neutrophil transmigration via myosin light chain kinase-dependent cell contraction. Blood 118, 1632-1640. doi:10.1182/blood-2010-11-321125
Suowen, X., Iqra, I., Peter, J. L., Hong, L., Danielle, K., Xueying, Z., et al. (2021). Endothelial dysfunction in atherosclerotic cardiovascular diseases and beyond: from mechanism to pharmacotherapies. Pharmacol. Rev. 73, 924-967. doi:10.1124/pharmrev.120.000096
Tarbell, J. M., and Cancel, L. M. (2016). The glycocalyx and its significance in human medicine. J. Intern. Med. 280, 97-113. doi:10.1111/joim.12465
Tarbell, J. M., and Pahakis, M. Y. (2006). Mechanotransduction and the glycocalyx. J. Intern Med. 259, 339-350. doi:10.1111/j.1365-2796.2006.01620.x
Tarbell, J. M., Simon, S. I., and Curry, F.-R. E. (2014). Mechanosensing at the vascular interface. Annu. Rev. Biomed. Eng. 16, 505-532. doi:10.1146/annurev-bioeng-071813-104908
Tegos, T. J., Kalodiki, E., Sabetai, M. M., and Nicolaides, A. N. (2001). The genesis of atherosclerosis and risk factors: a review. Angiology 52, 89-98. doi:10.1177/000331970105200201
Topper, J. N., Cai, J., Falb, D., and Gimbrone, M. A. (1996). Identification of vascular endothelial genes differentially responsive to fluid mechanical stimuli: cyclooxygenase-2, manganese superoxide dismutase, and endothelial cell nitric oxide synthase are selectively up-regulated by steady laminar shear stress. Proc. Natl. Acad. Sci. 93, 10417-10422. doi:10.1073/pnas.93.19.10417
Tzanatos, H. A., Tseke, P. P., Pipili, C., Retsa, K., Skoutelis, G., and Grapsa, E. (2009). Cardiovascular risk factors in non-diabetic hemodialysis patients: a comparative study. Ren. Fail. 31, 91-97. doi:10.1080/08860220802595484
Van Den Bulcke, A. I., Bogdanov, B., De Rooze, N., Schacht, E. H., Cornelissen, M., and Berghmans, H. (2000). Structural and rheological properties of methacrylamide modified gelatin hydrogels. Biomacromolecules 1, 31-38. doi:10.1021/bm990017d
Virani, S. S., Alonso, A., Aparicio, H. J., Benjamin, E. J., Bittencourt, M. S., Callaway, C. W., et al. (2021). Heart disease and stroke statistics-2021 update: a report from the American heart association. Circulation 143, e254-e743. doi:10.1161/cir.0000000000000950
Vlahu, C. A., Lemkes, B. A., Struijk, D. G., Koopman, M. G., Krediet, R. T., and Vink, H. (2012). Damage of the endothelial glycocalyx in dialysis patients. J. Am. Soc. Nephrol. 23, 1900-1908. doi:10.1681/asn.2011121181
Wallach, D. F., and Kamat, V. B. (1966). The contribution of sialic acid to the surface change of fragments of plasma membrane and endoplasmic reticulum. J. Cell Biol. 30, 660-663. doi:10.1083/jcb.30.3.660
Yao, Y., Rabodzey, A., and Dewey J R, C. F. (2007). Glycocalyx modulates the motility and proliferative response of vascular endothelium to fluid shear stress. Am. J. Physiology-Heart Circulatory Physiology 293, H1023-H1030. doi:10.1152/ajpheart.00162.2007
Yeh, Y.-T., Hur, S. S., Chang, J., Wang, K.-C., Chiu, J.-J., Li, Y.-S., et al. (2012). Matrix stiffness regulates endothelial cell proliferation through septin 9. PLOS ONE 7, e46889. doi:10.1371/journal.pone.0046889
Yen, W., Cai, B., Yang, J., Zhang, L., Zeng, M., Tarbell, J. M., et al. (2015). Endothelial surface glycocalyx can regulate flow-induced nitric oxide production in microvessels in vivo. PLOS ONE 10, e0117133. doi:10.1371/journal.pone.0117133
Yue, K., Trujillo-De Santiago, G., Alvarez, M. M., Tamayol, A., Annabi, N., and Khademhosseini, A. (2015). Synthesis, properties, and biomedical applications of gelatin methacryloyl (GelMA) hydrogels. Biomaterials 73, 254-271. doi:10.1016/j.biomaterials.2015.08.045
Zeng, Y., Zhang, X. F., Fu, B. M., and Tarbell, J. M. (2018). “The role of endothelial surface glycocalyx in mechanosensing and transduction,” in Molecular, cellular, and tissue engineering of the vascular system. Editors B. M. FU, and N. T. WRIGHT (Cham: Springer International Publishing).
Zhong, A., Mirzaei, Z., and Simmons, C. A. (2018). The roles of matrix stiffness and βcatenin signaling in endothelial-to-mesenchymal transition of aortic valve endothelial cells. Cardiovasc. Eng. Technol. 9, 158-167. doi:10.1007/s13239-018-0363-0
Zhu, M., Wang, Y., Ferracci, G., Zheng, J., Cho, N.-J., and Lee, B. H. (2019). Gelatin methacryloyl and its hydrogels with an exceptional degree of controllability and batchto-batch consistency. Sci. Rep. 9, 6863. doi:10.1038/s41598-019-42186-x

### Example 2

Example 2 presents an innovative in vitro model aimed at investigating the combined effects of tissue rigidity and shear stress on endothelial cell (EC) function, which are crucial for understanding vascular health and the onset of diseases such as atherosclerosis. Traditionally, studies have explored the impacts of shear stress and substrate stiffness on ECs, independently. However, this integrated system combines these factors to provide a more precise simulation of the mechanical environment of the vasculature. The objective is to examine EC mechanotransduction across various tissue stiffness levels and flow conditions using human ECs. Example 2 details the protocol for synthesizing gelatin methacrylate (GelMA) hydrogels with tunable stiffness and seeding them with ECs to achieve confluency. Additionally, Example 2 describes the design and assembly of a cost-effective flow chamber, supplemented by computational fluid dynamics simulations, to generate physiological flow conditions characterized by laminar flow and appropriate shear stress levels. The protocol also incorporates fluorescence labeling for confocal microscopy, enabling the assessment of EC responses to both tissue compliance and flow conditions. By subjecting cultured ECs to multiple integrated mechanical stimuli, this model enables comprehensive investigations into how factors such as hypertension and aging may affect EC function and EC-mediated vascular diseases. The insights gained from these investigations will be instrumental in elucidating the mechanisms underlying vascular diseases and in developing effective treatment strategies.

Endothelium, lining the inner surface of blood vessels, plays a pivotal role in maintaining vascular health. Endothelial cells (ECs) are central to regulating various cardiovascular functions, including vessel tone control, selective permeability, hemostasis, and mechanotransduction^{1, 2}. Research has firmly linked EC dysfunction to a primary role in atherosclerosis development. Notably, ECs encounter diverse mechanical forces at the interfaces where they interact with blood flow and underlying vessel tissues^{3, 4}. Several studies have associated EC dysfunction with abnormal changes in mechanical factors within the vascular environment, such as the fluid shear stress from blood flow and tissue rigidity^{5, 6-7}.

However, prior research has received limited attention in comprehending the combined effects of tissue rigidity and shear stress on EC function. To enhance the ability to translate research outcomes into effective treatments for atherosclerosis and other cardiovascular diseases, it is essential to improve the cellular models used in the field. Significant progress has been made in humanizing cellular models by employing human ECs and subjecting them to either shear stress or substrates with varying stiffness levels^{8, 9, 10}. However, the adoption and refinement of cellular models that integrate dynamic flow environments with EC substrates possessing adjustable stiffness properties has progressed slowly. The challenge lies in devising non-swelling EC substrates to prevent alterations in flow parameters within the flow channel while also facilitating the cultivation of intact and well-adhered EC monolayers. An in vitro model capable of overcoming these obstacles could facilitate more effective investigations into how hypertension, aging, and flow conditions collaboratively influence EC mechanotransduction, vascular health, and, ultimately, the development of atherosclerosis. Various methods have been developed to apply shear stress on cells while controlling substrate stiffness, including rotating plates and microfluidic devices. In the rotating plate method, cells are placed between two plates and shear stress is applied through the rotational movement of the plates. This method is less complicated and provides a quick model; however, it suffers from spatial shear stress variation, with zero shear stress at the center and maximum shear stress at the periphery¹¹.

On the other hand, microfluidic devices represent the new generation of tools with the ability to control substrate rigidity and flow conditions. These systems are suitable for mimicking microvasculatures under laminar flow conditions. However, studying atherosclerosis with such devices is impractical, as atherosclerosis occurs in large vessels with disturbed flow¹¹. This disclosure aims to contribute to the critical research domain of EC studies by presenting a cost-effective system capable of examining the effects of varying stiffness levels in EC substrates under different flow conditions. The system integrates substrates with different stiffnesses to emulate pathological and physiological blood vessels. This protocol outlines the method for creating gelatin-based hydrogels with no swelling and stiffness levels of 5 kPa and 10 kPa, representing physiological and pathological stiffness, respectively. Additionally, the construction of a parallel-plate flow chamber capable of integrating these substrates is detailed. Computational fluid dynamics (CFD) was employed to evaluate shear stress and flow conditions. The preparation of hydrogels for EC culture and the execution of a 6 h flow experiment are described, followed by a discussion on post experiment immunostaining.

### Synthesis of GelMA

1. Prepare a 0.2 M solution of anhydrous sodium carbonate and a 0.2 M solution of sodium bicarbonate.

2. Mix 46 mL of sodium bicarbonate solution with 15 mL of sodium carbonate solution and add 139 mL of deionized (DI) water. Adjust the pH to 9.5 using 0.1 M NaOH and HCl if necessary.

3. Add 10 g of type A, 300-bloom gelatin from porcine skin to 100 mL of carbonate-bicarbonate buffer at a concentration of 10% w/v.

4. Dissolve the gelatin 551 using a 55° C. water bath while stirring the solution at 700 rpm using a magnetic stirrer. Once fully dissolved, adjust the pH of the gelatin solution to 9.5 using 0.1 M NaOH.

5. To initiate methacrylation, add 938 μL of methacrylic anhydride (MAH) dropwise to the solution. Improve the initial distribution of MAH in the gelatin solution by injecting it at various locations, including different depths and radial distances from the center.

6. Wrap the reaction vessel in aluminum foil to prevent exposure to light. Maintain the temperature at 55° C. and stir the solution at 500 rpm for 1 h to complete the reaction (FIG. 5)¹².

NOTE: The reaction stops at a pH below 7.4.

7. Prepare one 2 L beaker containing 1.8 L of acetone and one 0.6 L beaker containing 0.3 L of acetone.

8. Add the resulting GelMA solution dropwise to the 2 L beaker while stirring at 200 RPM to induce precipitation¹³. To maximize the collection of the precipitated product, place a stainless-steel rod or spatula as a nucleation site for easier transfer.

9. Transfer the product from the larger beaker to the smaller one and allow it to sit for 10 min before proceeding to the drying step. The precipitated GelMA should appear as white fibers.

10. Collect the product on absorbent paper, dry it in a vacuum oven at room temperature (RT), and store it at −20° C. until further use.

NOTE: Additional details on the chemical characterization of the product via proton nuclear magnetic resonance (1H NMR) can be found in a previously published work⁸.

### Glass Salinization

NOTE: Attaching hydrogels to glass slides 552 provides a flat and even surface, facilitating handling and ensuring stability under flow-derived shear stress. Functionalizing the glass with 3-(trimethoxysilyl)propyl methacrylate is necessary to enhance surface properties and enable the covalent attachment of hydrogels during the polymerization process.

1. Precisely cut plain microscope slides (1 mm thickness) into pieces similar to the final hydrogel dimensions using a glass cutter. Wash the cut slides with soap to remove surface contaminants and debris that could obstruct the treatment of the glass surface.

NOTE: If necessary, sonicate the glass slides for 5 min in ethanol, then air-dry.

2. Prepare a 0.5% solution of 3-(trimethoxysilyl)propyl methacrylate in absolute ethanol. Prepare a 10% glacial acetic acid solution in DI water. Mix the solutions to achieve a final concentration of 3% glacial acetic acid.

NOTE: The glacial acetic acid solution can be prepared in large quantities and stored.

3. Organize the glass slides in a glass container to ensure the glass surfaces are not obstructed. Pour the resulting solution over the glass slides and keep them for approximately 5 min on a rocker at 80 rpm for the reaction to complete. Ensure both sides of the glass slides are modified by removing any trapped air bubbles.

4. After the reaction is complete, aspirate the solution and wash the glass slides with ethanol 2×. Air dry the slides and store them in the dark at RT.

NOTE: The glass slides retain their modification for 1 month under these conditions.

### Hydrogel Preparation

1. Fabricate hydrogels 553a using a redox-induced free radical polymerization method.

1. Assemble two-piece Polytetrafluoroethylene (PTFE) molds with a proper depth and opening windows of 10 mm² to shape the final substrate. Account for a 25% shrinkage in the hydrogel's height post polymerization during the design process. Ensure the molds are flat and are tightly fastened at the bottom and top plates to prevent leakage.

NOTE: The designed mold dimensions are intentionally 10% larger than the intended hydrogel size. This design serves multiple purposes: it prevents damage to the sides upon mold separation, enhances surface evenness of the hydrogel by pushing impurities or bubbles to the sides, and compensates for the anticipated 25% shrinkage after equilibration due to the syneresis effect, where highly crosslinked hydrogels repel water post-equilibration, causing shrinkage. The shrinkage amount is specified in the protocol⁸.

2. To ensure hydrogels have an even surface and constant height, suspend modified glass slides from the top plate of the mold, allowing a narrow opening for injecting the polymer solution. Use a cover glass to suspend the modified glass slides (FIG. 5).

3. To create the cover glass, cut plain microscopic glass 20% larger than the mold. Attach two spacers to the shorter sides. Calculate the spacer thickness as follows:

Spacer thickness=1.33×final gel thickness+1 (thickness of the modified glass)−depth of the mold

4. Apply a thin layer of cell-compatible sealing grease to the longer sides of the cover glass, on the same face where the spacers are attached.

5. Attach the modified glass slide to the cover glass between the two spacers.

6. Place the cover glass on the mold so that the spacers sit on the mold and the modified glass extends into the mold. This setup provides a clearance of 1.33 of the final hydrogel's height between the modified glass and the bottom of the mold.

7. Dissolve GelMA in DI water at 4% and 10% w/v for 5 kPa and 10 kPa hydrogels, respectively, and place it in a 45° C. water bath.

NOTE: GelMA is a thermosensitive polymer and maintaining the solution temperature at 45° C. prevents solidification before polymerization and reduces solution viscosity, which is useful for fabricating flat hydrogels.

8. Add TEMED (24 mM for 5 kPA hydrogel, 6.25 mM for 10 kPa hydrogel) to the polymer solution and mix thoroughly.

NOTE: TEMED alone does not initiate polymerization, so ensure molds, pipettes, and solutions are prepared before proceeding.

9. Add APS (24 mM for 5 kPA hydrogel, 24.5 mM for 10 kPa hydrogel) to the GelMA solution and mix thoroughly.

NOTE: Adding APS initiates the polymerization, and hydrogels may form quickly, requiring prompt action to prevent defective hydrogels. Further details on stiffness measurements can be found in a previously published work⁸.

10. Carefully pipette the resulting solution to the opening between the suspended glass slide and the mold at RT and allow it to crosslink. Capillary force drives the polymer solution into the mold. Avoid adding bubbles to the gels and stop pipetting when a small amount of solution remains in the pipette tip.

NOTE: During polymerization, methacrylate groups of GelMA react with methacrylate residues on the modified glass slides, resulting in the chemical attachment of the hydrogel to the glass.

11. After 15 min, the reaction is complete. Detach the substrate from the mold using a sharp object, such as a needle, and slide the substrate away from the cover glass to separate it.

12. Transfer the hydrogels to 1× phosphate-buffered saline (PBS) in a 100 mm Petri dish sealed with plastic film and equilibrate at 37° C. to remove remaining reactants and byproducts.

13. As the hydrogel is slightly larger than the final dimensions, trim any excess gel at the sides to match the dimensions required for the flow chamber window.

14. Use the flow chamber to verify the hydrogel dimensions. Ensure the hydrogel fits properly in the flow chamber without gaps between the hydrogel and the mold or any defects that could affect flow patterns. If there are shape irregularities that may affect flow pattern in the flow chamber, leading to adverse cell behavior, save the hydrogel for static conditions.

15. Transfer four hydrogels to a Petri dish containing 1×PBS for sterilization.

2. Sterilize the hydrogel as described below using 70% ethanol.

1. Prepare 25%, 50%, and 70% alcohol solutions for gradual hydrogel dehydration.

NOTE: If gradual dehydration is not carried out, stiffer hydrogels may shrink and break, while wrinkles may form on the surface of softer hydrogels.

2. Aspirate the 1×PBS solution from the Petri dish containing hydrogels. Add 25% ethanol solution to each Petri dish to submerge the 5 kPa and 10 kPa hydrogels for 15 min.

3. Aspirate the 25% alcohol solution. Add 50% alcohol solution to each Petri dish to submerge the 5 kPa and 10 kPa hydrogels for 15 min.

4. Aspirate the 50% alcohol solution. Add 70% alcohol solution to each Petri dish, submerging the 5 kPa and 10 kPa hydrogels for 5 min. Place the Petri dish on a shaker at 100 rpm.

5. Leave the 5 kPa hydrogels submerged for 40 min and the 10 kPa hydrogels for 20 min.

NOTE: The 5 kPa hydrogels were observed to be more prone to contamination compared to 10 kPa hydrogels.

6. Transfer the samples to a 6-well plate in a biosafety cabinet and wash the hydrogels 2×-3× using sterile PBS.

4. Coating hydrogels

1. Prepare a 60 μg/mL gelatin solution by dissolving 3 mg of gelatin in 50 mL of sterile PBS (1× with magnesium and calcium salt). Heat the solution in a water bath at 37° C. for 30 min or until all gelatin is fully dissolved.

2. Sterile filter the coating solution using a 0.2 μm syringe filter. Aspirate the 1×PBS solution from each well plate and add gelatin solution to each well, ensuring complete coverage of each hydrogel. To minimize the risk of contamination, add 40 units/mL of penicillin and 10 μg/mL of streptomycin to the coating solution.

3. Incubate each well plate in a 37° C., 5% CO2 incubator for 45 min. Wash the hydrogels with 1×PBS solution.

4. Aspirate PBS and add cell culture media.

5. For no longer than two days, keep the hydrogels in cell culture media and incubate them in a 37° C., 5% CO2 incubator until cell seeding.

NOTE: Despite fibronectin's excellent cell attachment properties, there is a concern about its use due to ECs depositing endogenous fibronectin in atherosclerotic conditions, which can lead to a proinflammatory response. To avoid chemically stimulated cell responses, fibronectin was not used. Additionally, Orr et al. demonstrated that fibronectin coating, compared to collagen I coating, upregulated atherogenic genes.

Specifically, they observed increased expression levels of intercellular adhesion molecule 1 (ICAM-1), vascular cell adhesion molecule 1 (VCAM-1), and Nuclear factor-κB (NF-κB)¹⁴.

5. Seeding cells on the substrates

1. Prepare the cell suspension following available detachment protocols and count the cells using a hemocytometer⁸.

2. Thoroughly aspirate the media from the hydrogels. Add 50,000 cells/cm² to each sample. Add a proper volume of cell suspension to each sample to prevent cell overflow.

NOTE: Stiffer substrates are more hydrophobic; therefore, minimize the time between steps to improve wettability and cell suspension dispersion on the substrate surface.

3. Incubate cell-seeded hydrogels 553b in a 37° C., 5% CO₂ incubator for 2 h. Add cell culture media to the samples once the cells are attached to the gels.

6. Flow chamber fabrication

NOTE: The approach for designing the flow chamber is cost effective and requires minimal expertise for fabrication and utilization.

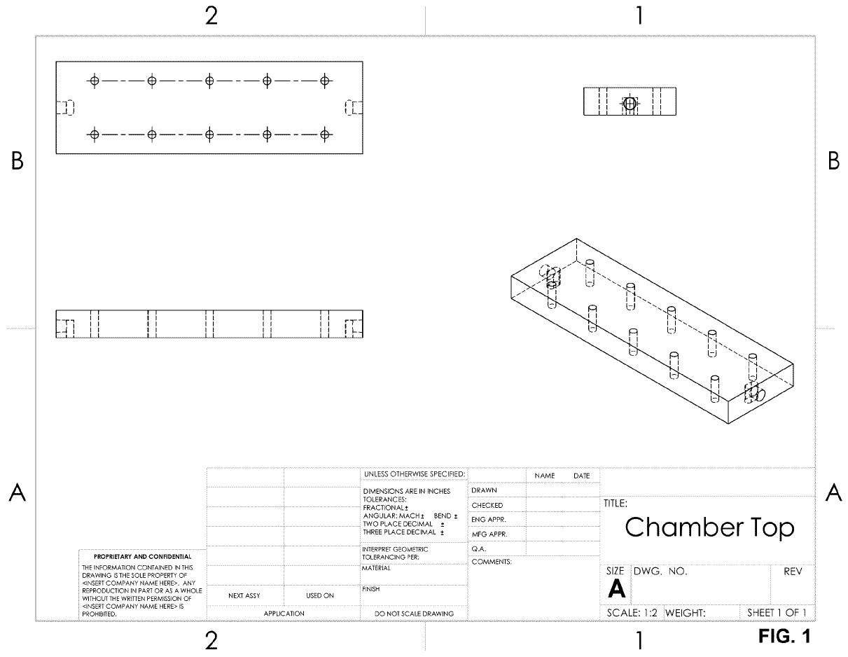

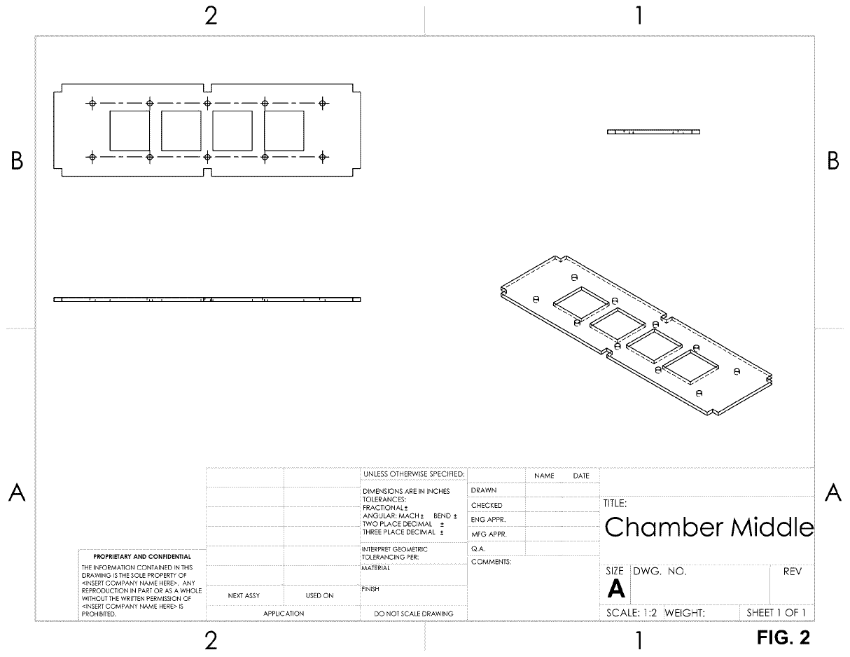

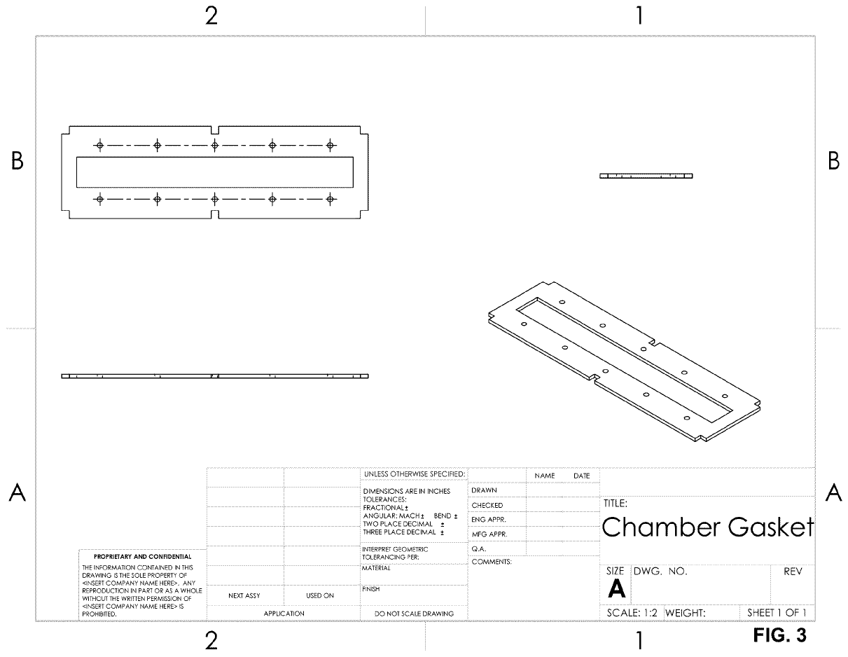

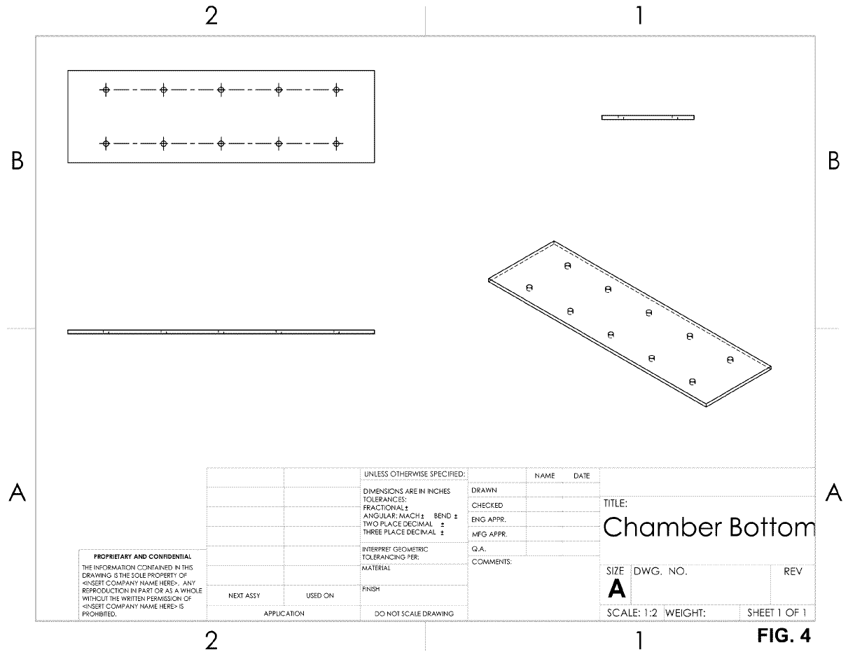

1. Use poly(methyl methacrylate) for chamber fabrication to visually assess flow quality, hydrogel integrity under shear stress, and potential real-time biomarker studies during flow experiments. The chamber design was created using computer-aided design (CAD) software (FIGS. 1-4).

2. Computational assessment of flow conditions in the flow chamber

1. Assemble the individual components of the designed flow chamber (. SLDPRT files) to create the final chamber setup 554a.

2. Draw a line along the centerline of the flow tangential to the hydrogel surface to analyze shear stress. Close the inlet and outlet openings to define a closed control volume.

3. Open Flow Simulation from the Add-Ins tab in the CAD software. In the Flow Simulation tab, open the Wizard feature to enter the properties. Specify the unit system and adjust pressure and the stress units to dyne/cm².

4. Check Fluid Flow and Gravity in the Physical Features. Set gravity to −9.8 in Z component; X and Y components should be zero.

5. Choose Internal for analysis type in Geometry Handling. Select Water as the default fluid.

NOTE: Assume the medium behaves like water.

6. Choose Laminar and Turbulent for the flow type. Define the wall as an adiabatic wall and input surface roughness in Wall Condition.

7. Set the temperature to 37.5° C. (310.65 K) in Initial Conditions. Define the computational domain in Flow Simulation projects, ensuring it covers the entire control volume.

8. Select all walls interfacing with the fluid to define the fluid subdomain. In Boundary Condition, designate the inner surface of the inlet lid as Inlet Volume Flow and specify flow rate (Q) and uniform flow. Then, assign the inner surface of the outlet lid sealing as static pressure.

9. Determine the Global Mesh Size based on available computational resources. Hit Run to initiate the simulation.

10. Upon completion, review desired results, including shear stress plots and flow simulations 554b.

11. Repeat steps 6.2.8 to 6.2.10 with various flow rates to calculate the applied shear stress at different rates. Use regression analysis to establish the flow rate-shear stress relationship.

12. Assess the system's tolerance for variations in hydrogel dimensions to enhance the simulation's realism.

7. Run uniform laminar flow

1. After culturing cells on the 5 kPa and 10 kPa hydrogels, ensure confluence of the cell monolayer in a 37° C., 5% CO₂ incubator. A monolayer of cells on each hydrogel should be achieved.

2. Sterilize autoclavable components of the parallel plate flow chamber system (stainless steel screws, spatula, forceps, gasket, and the media reservoir) with a 30 min gravity autoclave cycle. Sterilize other parts (acrylic components and reservoir sealer/dampers) with UV light exposure.

3. Assemble the flow chamber device in a biosafety cabinet. Secure hydrogel samples in the device, ensuring uniformity. Use a filler of equal size to the hydrogels if an insufficient number of samples are available for flow.

4. Pre-wet the inner surface of the chamber and hydrogel surfaces to minimize bubble formation and prevent cell drying during assembly.

5. Add enough media to the inlet reservoir, allowing 20%-30% of the media to flow to the outlet reservoir upon assembly. Seal the inlet reservoir airtight using a damper/sealer.

NOTE: Pressure build-up in the inlet reservoir drives flow, and any air leaks alter flow rates. Check for sealer defects if bubbles form in the outer tubing.

6. Set the outlet damper to atmospheric pressure to establish the pressure difference. Place the device and reservoirs in a 37° C., 5% CO₂ incubator. Connect the device tubing to a peristaltic pump placed outside of the incubator 555 (FIG. 5).

7. Turn on the pump to start applying 3.6 dyne/cm². Gradually increase the flow rate by 2 mL/min increments (e.g., reach 85 mL/min from 65 mL/min after 10 min) until applying approximately 8 dyne/cm².

8. Further increase flow rate by 2.5 mL/min increments until reaching 12 dyne/cm² of shear stress.

NOTE: Allow time for cells to adapt to the dynamic condition, as rapid flow rate increases may detach cells and damage the monolayer.

9. After 6 h, stop the flow, remove the device from the incubator, and disassemble the flow chamber. Subsequently, transfer hydrogel samples to a six-well plate.

10. Wash the samples with ice-cold PBS and either fix the cells for immunostaining or lyse them for protein isolation.

8. Immunostaining setup for confocal microscopy with high magnification

NOTE: To increase study efficiency, a method was developed for immunostaining small portions of hydrogels, enabling the examination of multiple biological targets in a single sample.

1. Prepare biopsy punches or preferred cutting tools with diameters of 3 or 4 mm.

2. Use a pipette tip box as a staining chamber. Humidify the chamber to control staining solution evaporation during extended incubation by placing a wet paper towel at the bottom of the pipette tip box.

3. Reduce antibody consumption by creating small solution pools for individual samples. Cover the tip box rack with transparent film, gently pressing the film against the rack holes with a fingertip to make dimples. Fill the dimples with 70 L of PBS.

4. Transfer an individual hydrogel to an empty Petri dish and cut it using a biopsy punch or preferred cutting tool. Use a microscope to cut a representative sample area. Cut a full gel cylinder to avoid confocal microscopy issues.

5. Place the cut hydrogel samples into dimples created in step 8.3. Perform staining following standard protocol¹⁵.

After the final staining wash, obtain a silicon rubber sheet that matches the hydrogel thickness.

NOTE: Preferably, use a sheet with one sticky side for improved sealing. In this study, actin fibers were stained using a fluorescent secondary antibody conjugated to Phalloidin at a dilution of 1:20.

6. Cut the rubber sheet into 15 mm×15 mm squares. Punch a 6 mm hole using a biopsy punch or preferred cutting tool.

7. Create a sample container by attaching the rubber to a microscopy coverslip (no. 1.5). Add 10 μL of wet mounting media to the dimples while the sample is still present and incubate it in the dark for 10-15 min.

NOTE: For this study, the mounting media contained 0.9 μg/mL of 4′,6-diamidino-2-phenylindole (DAPI) to shorten the staining protocol.

8. Remove the sample from the staining chamber and place it in the container created in step 8.7. Apply 1-2 L of mounting media on top of the sample.

NOTE: The quantity of mounting media impacts image quality at high magnifications. Excessive or insufficient mounting media can compromise image clarity.

9. Position another coverslip on top and gently press to ensure the sample contacts the glass. Store the samples at 4° C. in the dark until microscopy imaging.

### Representative Results

FIG. 5 depicts the experimental setup 550, outlining the process of GelMA synthesis 551 through a methacrylation reaction. The resulting product was then attached to a modified microscope slide 552 and used to fabricate the hydrogel substrate 553a, onto which ECs were seeded 553b. Subsequently, the cells were introduced into the flow chamber 554a for a 6 h flow experiment at 12 dyne/cm² 555.

1H NMR spectroscopy was used to assess the success of the methacrylation reaction (FIG. 6). The presence of a methyl group at 1.9 ppm and a vinylic peak between 5.4-5.6 ppm in GelMA 661b confirmed successful methacrylation 661a. Additionally, the decrease in the lysine peak at 3 ppm 661c in GelMA indicates the consumption of lysine residues, which are replaced with methacrylate residues^{12, 13, 16}. The stiffness of GelMA hydrogels was evaluated using a compression test, which showed that the compression moduli increased with GelMA concentrations 662. Hydrogels composed of 4% and 8% (w/v) GelMA were used to mimic physiological (5 kPa) and pathological (10 kPa) matrix stiffnesses, respectively⁸.

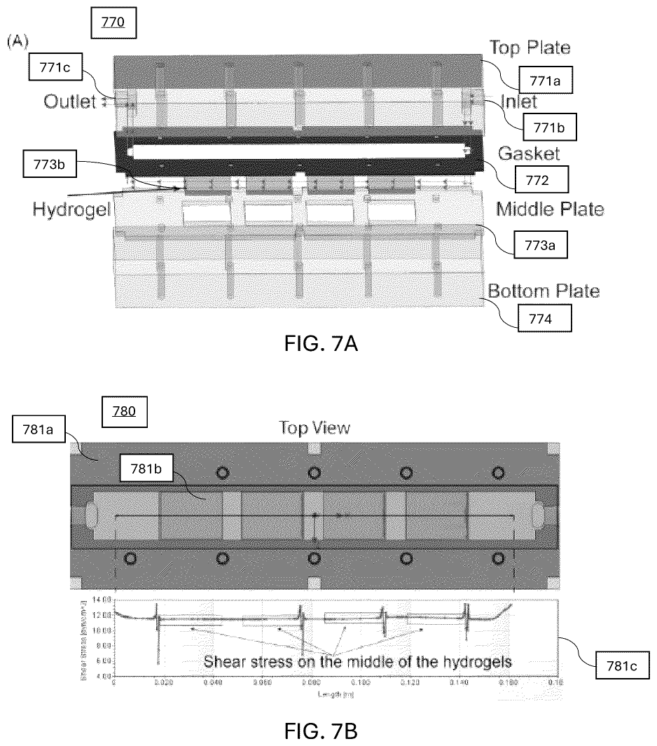

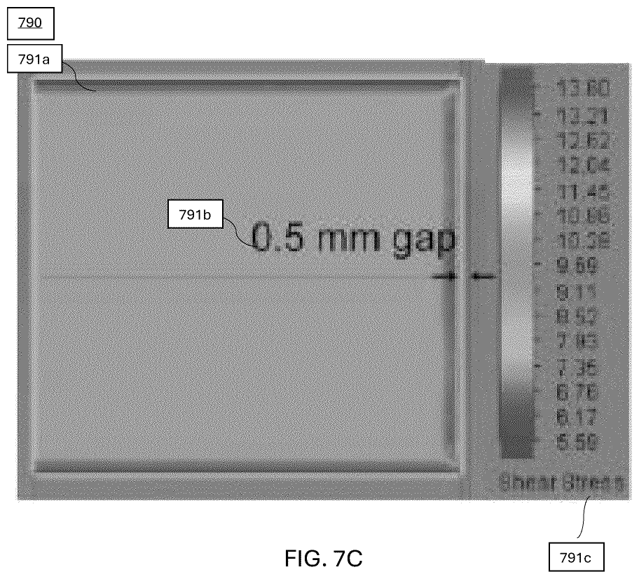

The flow chamber 770 was engineered to be cost-effective and for easy sterilization, by utilizing acrylic polymer that is UV-resistant. Its transparency facilitates real-time monitoring of hydrogels and flow conditions during experiments. Designed with three distinct layers, the chamber minimizes the risk of hydrogel damage during loading or unloading: the bottom plate 774 provides a sturdy base, the middle layer 773a offers lateral support for the hydrogels 773b, and the top plate 771a, along the gasket 772, creates the clearance necessary for fluid flow 771b and 771c (FIG. 7A). Computational simulations were conducted using CFD 780 to assess flow conditions and shear stress within the chamber. The following equation—shear stress=0.0558×flow rate—calculated the applied shear stress 781c to the cells based on the flow rate as an input (FIG. 7B). Notably, changes in material properties, such as stiffness, did not alter shear stress in the simulations. To count for differences in the hydrogel 78 lb size in the final experimental setup, the hydrogels 781b were intentionally sized slightly smaller in the computational model. A 0.5 mm gap 791b was created between one side of the hydrogels and the chamber's middle plate walls, perpendicular to the flow direction 781a. This configuration allowed for the analysis of shear stress effects 790 in these gaps 791a. While irregularities in shear stress were observed at gap locations (FIG. 7B), their impact was confined to a small area adjacent to gaps, with the remaining hydrogel surface experiencing uniform shear stress 791c (FIG. 7C). These insights suggest discarding cells from the edges of hydrogels to minimize the potential impact of turbulent regions. It is worth mentioning that higher shear stress, up to 15 dyne/cm², was experimentally applied to ECs seeded on 5 kPa and 10 kPa hydrogels with no leakage in the device. However, increasing shear stress further could potentially result in cell detachment and hydrogel failure, emphasizing the need for careful optimization of experimental conditions.

For seeding cells to form a monolayer, it is useful to use a higher cell density than in traditional cultures. Low seeding density has been shown to hinder the monolayer formation on softer hydrogels⁸. Additionally, pre-coating hydrogels with gelatin before cell seeding enhances initial cell attachment and spreading on softer hydrogels. However, it is important to note that the beneficial effect of this coating is temporary, as it primarily facilitates the initial interaction between the cells and the substrate.

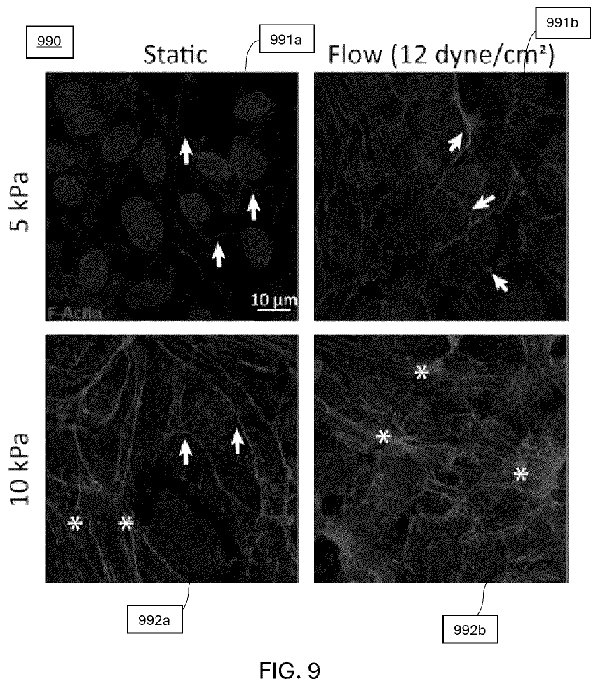

FIG. 9 demonstrates how stiffness and shear stress influence the formation of actin fibers 990. Under shear stress, thicker stress fibers formed, suggesting a stronger attachment to the surface. In softer samples 991a and 991b, there were more peripheral actin fibers, which are indicators of physiological conditions. However, in ECs on stiffer substrates 992a and 992b, the presence of stronger stress fibers and fewer peripheral fibers could potentially lead to EC dysfunction¹⁷. This data confirms the effectiveness of the presented system in modulating EC behavior.

### Discussion

The vascular system is a dynamic environment where various forces significantly influence cellular behavior. Studying biological events in cardiovascular diseases without considering these forces would be inaccurate. Thus, cellular models capable of emulating the vascular mechanical environment are useful. Researchers have already made significant progress in highlighting the effect of these forces on cellular behavior¹¹. However, to understand cell behavior under both pathological and physiological conditions in the human body, it is essential to develop more precise models that more closely resemble the blood vessel's environment.

Therefore, Example 2 aimed to develop a system that more accurately replicates the blood vessel environment while maintaining ease of access and user-friendliness.

The model can apply controlled flow-derived shear stress to human cells on substrates with varying stiffness levels, producing conditions closer to physiological realities compared to existing models. GelMA was synthesized and utilized in this model to meet the following criteria: 1) tunable mechanical properties, 2) non-swelling behavior, 3) cell compatibility and adhesion, and 4) the capability of embedding vascular cells to model the blood vessels more accurately. The adjustability of mechanical properties was achieved by varying the biopolymer concentration8 to mimic physiological and pathological conditions. The second criterion was the non-swelling behavior. It is useful to have a non-swelling substrate to maintain consistent flow chamber dimensions, related flow conditions, and shear stress on the cells. GelMA with a high degree of methacrylation demonstrated non-swelling properties, preserving the hydrogel's shape and surface smoothness throughout the experiment⁸. Importantly, the concentration and stiffness did not affect the swelling behavior, which simplified the model by eliminating the need for separate adjustments for each experimental group. The third criterion was cell adhesion, as proper attachment is necessary to prevent cell detachment and preserve monolayer integrity. GelMA provided cell adhesion, thereby reducing the need for additional steps to conjugate cell-adhesive molecules to the substrate, which is essential for many biopolymers. Furthermore, GelMA's capability for cell encapsulation was considered, although it was not directly tested in this study. The cell encapsulation potential has indications for supporting 3D cell culture and integrating layers of cells, such as vascular smooth muscle cells or pericytes, to enhance the model's accuracy¹⁹. In addition, the synthesis of GelMA is cost-effective and requires minimal equipment, making it an excellent candidate as a biomaterial for substratefabrication^{20, 21,22}

The parallel-plate flow chamber is commonly used to apply shear stress to cells, but it has traditionally only been used with glass coverslips or rigid materials. However, such materials lack physiological relevance²³. In contrast, microfluidic devices have introduced more geometric complexity and softer substrates by utilizing polymer-based materials. However, these devices often cannot control the flow regime accurately, and their small dimensions limit their capacity to studying only a small number of cells, limiting experimental outcomes¹¹. The proposed device combines the benefits of both systems by integrating endothelial cell monolayer seeded hydrogels with a flow chamber applying precisely controlled shear stress.

The device has demonstrated the capability to integrate both flow-derived and solid-derived mechanical forces. When a shear stress of 12 dyne/cm² was applied for 6 h, a formation of cytosolic stress fibers was observed, contrasting with the predominance of peripheral actin in the softer substrate group. This is in line with many reports showing fewer stress fibers formed when ECs are cultured on softer surfaces^{24, 25, 26, 27}. On the other hand, laminar flow could result in prominent stress fiber formation. It has been shown that cytoskeletal response to flow conditions starts within 1 h of exposure to the flow but requires a remarkably longer time to complete reorganization^{28, 29, 30}. The peripheral actin network is essential for various EC functions, including cell-cell adhesion and barrier functionality¹⁷. Upregulating this network in a healthy experimental group in comparison to the pathologic group with extensive stress fibers approves the device's successful modeling of healthy and diseased conditions.

One drawback of this device is the potential for damage to the hydrogels, which could disrupt the flow and diminish the success rate of experiments. This issue primarily arises from initial defects in the hydrogels, which, under shear stress, may worsen, leading to the detachment of the sample and partial flow obstruction. Therefore, the sample preparation steps, including polymerization, equilibration, and cutting, should be conducted carefully to prevent any additional damage to the samples. Another challenge in this system is achieving and maintaining the integrity of the monolayer. While coating the hydrogels with gelatin can improve initial cell attachment, previous work showed that this coating does not affect cell proliferation⁸. Therefore, to enhance monolayer formation, especially considering that cell proliferation is slower on softer hydrogels³¹, increasing the seeding density is beneficial. Additionally, the cells may detach due to the shear stress induced by fluid flow. Hence, it is useful to gradually increase the flow rate, allowing the cells sufficient time to adapt to the new environmental conditions.

In conclusion, the device represents a significant advancement in simulating the vascular environment more accurately due to its ability to simultaneously simulate both fluid-derived and solid-derived mechanical forces. It offers a comprehensive platform for studying EC behavior under various physiological and pathological conditions. This versatility makes it a valuable tool for advancing the understanding of vascular biology and disease progression. This model can contribute to a variety of research studies, including mechanobiology, atherosclerosis, cancer metastasis development, vascular tissue engineering and angiogenesis, and drug delivery and screening.

### REFERENCES

1. Deanfield, J. E., Halcox, J. P., Rabelink, T. J. Endothelial function and dysfunction. Circulation. 115 (10), 1285-1295 (2007).
2. Suowen, X. et al. Endothelial dysfunction in atherosclerotic cardiovascular diseases and beyond: From mechanism to pharmacotherapies. Pharmacol Rev. 73 (3), 924 (2021).
3. Jansen, K. A., Atherton, P., Ballestrem, C. Mechanotransduction at the cell-matrix interface. Sem Cel Dev Biol. 71, 75-83 (2017).
4. Tarbell, J. M., Pahakis, M. Y. Mechanotransduction and the glycocalyx. J Int Med. 259 (4), 339-350 (2006).
5. Topper, J. N., Cai, J., Falb, D., Gimbrone, M. A. Identification of vascular endothelial genes differentially responsive to fluid mechanical stimuli: cyclooxygenase-2, manganese superoxide dismutase, and endothelial cell nitric oxide synthase are selectively up-regulated by steady laminar shear stress. Proc Natl Acad Sci. 93 (19), 10417-10422 (1996).
6. Mitchell, G. F. et al. Arterial stiffness and cardiovascular events. Circulation. 121 (4), 505-511 (2010).
7. Bonetti, P. O., Lerman, L. O., Lerman, A. Endothelial dysfunction. Arterioscler Thromb Vasc Biol. 23 (2), 168-175 (2003).
8. Hamrangsekachaee, M. et al. Endothelial glycocalyx sensitivity to chemical and mechanical sub-endothelial substrate properties. Front Bioeng Biotechnol. 11, 1250348 (2023).
9. Mensah, S. A. et al. Flow-regulated endothelial glycocalyx determines metastatic cancer cell activity. FASEB J. 34 (5), 6166-6184 (2020).
10. Russell-Puleri, S. et al. Fluid shear stress induces upregulation of COX-2 and PGI2 release in endothelial cells via a pathway involving PECAM-1, PI3K, FAK, and p38. Am J Physiol-Heart Cir Physiol. 312 (3), H485-H500 (2016).
11. Hamrangsekachaee, M., Wen, K., Bencherif, S. A., Ebong, E. E. Atherosclerosis and endothelial mechanotransduction: current knowledge and models for future research. Am J Physiol-Cell Physiol. 324 (2), C488-C504 (2022).
12. Zhu, M. et al. Gelatin methacryloyl and its hydrogels with an exceptional degree of controllability and batchto-batch consistency. Sci Rep. 9 (1), 6863 (2019).
13. Kim, J., Bencherif, S. A., Li, W. A., Mooney, D. J. Cell-friendly inverse opal-like hydrogels for a spatially separated co-culture system. Macromol Rapid Comm. 35 (18), 1578-1586 (2014).
14. Orr, A. W. et al. The subendothelial extracellular matrix modulates NF-kappaB activation by flow: a potential role in atherosclerosis. J Cell Biol. 169 (1), 191-202 (2005).
15. Hamrangsekachaee, M., Baumann, H. J., Pukale, D. D., Shriver, L. P., Leipzig, N. D. Investigating mechanisms of subcutaneous preconditioning incubation for neural stem cell embedded hydrogels. ACS Appl Bio Mater. 5 (5), 2176-2184 (2022).
16. Rezaeeyazdi, M., Colombani, T., Memic, A., Bencherif, S. A. Injectable hyaluronic acid-co-gelatin cryogels for tissue-engineering applications. Materials. 11 (8), 1374 (2018).
17. Belvitch, P., Htwe, Y. M., Brown, M. E., Dudek, S. Cortical actin dynamics in endothelial permeability. Curr Top Membr. 82, 141-195 (2018).
18. Krishnamoorthy, S., Noorani, B., Xu, C. Effects of encapsulated cells on the physical-mechanical properties and microstructure of gelatin methacrylate hydrogels. Int J Mol Sci. 20 (20), 5061 (2019).
19. Jin, Q. et al. Bioprinting small-diameter vascular vessel with endothelium and smooth muscle by the approach of two-step crosslinking process. Biotechnol Bioeng. 119 (6), 1673-1684 (2022).
20. Villard, P. et al. Autoclavable and injectable cryogels for biomedical applications. Adv Healthcare Mater. 8 (17), 1900679 (2019).
21. Bencherif, S. A. et al. Influence of cross-linker chemistry on release kinetics of PEG-co-PGA hydrogels. J Biomed Mater Res A. 90 (1), 142-153 (2009).
22. Rogers, Z. J., Zeevi, M. P., Koppes, R., Bencherif, S. A. Electroconductive hydrogels for tissue engineering: Current status and future perspectives. Bioelectricity. 2 (3), 279-292 (2020).
23. James, B. D., Allen, J. B. Vascular endothelial cell behavior in complex mechanical microenvironments. ACS Biomater Sci Eng. 4 (11), 3818-3842 (2018).
24. Yeh, Y. T. et al. Matrix stiffness regulates endothelial cell proliferation through septin 9. PLoS One. 7 (10), e4688 (2012).
25. Fioretta, E. S., Fledderus, J. O., Baaijens, F. P. T., Bouten, C. V. C. Influence of substrate stiffness on circulating progenitor cell fate. J Biomech. 45 (5), 736-744 (2012).
26. Geemen, D. v. et al. F-Actin-anchored focal adhesions distinguish endothelial phenotypes of human arteries and veins. Arterioscler Thromb Vasc Biol. 34 (9), 2059-2067 (2014).
27. Dessalles, C. A., Leclech, C., Castagnino, A., Barakat, A. I. Integration of substrate- and flow-derived stresses in endothelial cell mechanobiology. Comm Biol. 4 (1), 764 (2021).
28. Estrada, R., Giridharan, G. A., Nguyen, M. D., Prabhu, S. D., Sethu, P. Microfluidic endothelial cell culture model to replicate disturbed flow conditions seen in atherosclerosis susceptible regions. Biomicrofluidics. 5 (3), 32006-3200611 (2011).
29. Inglebert, M. et al. The effect of shear stress reduction on endothelial cells: A microfluidic study of the actin cytoskeleton. Biomicrofluidics. 14 (2), 024115 (2020).
30. Noria, S. et al. Assembly and reorientation of stress fibers drives morphological changes to endothelial cells exposed to shear stress. Am J Pathol. 164 (4), 1211-1223 (2004).
31. Amer, M., Shi, L., Wolfenson, H. The ‘Yin and Yang’ of cancer cell growth and mechanosensing. Cancers (Basel). 13 (19), 4754 (2021).

### Example 3

The glycocalyx (GCX), a multi-component coating on endothelial cells (ECs), plays a critical role in various cellular behaviors, including barrier formation, vasodilation, and mechanotransduction. Mechanical perturbations in the vascular environment, such as blood vessel stiffness, are sensed and transduced by ECs via the GCX. Hypertension-induced stiffness disrupts GCX-mediated mechanotransduction, leading to EC dysfunction and atherosclerotic cardiovascular diseases. Understanding GCX-regulated mechanotransduction necessitates an in vitro model that closely mimics in vivo conditions. Existing models are insufficient, prompting the development of the system described in this manuscript. Here, reports on a new system to model varying EC substrate stiffness under sustained physiological fluid shear stress, providing a realistic environment for comprehensive examination of EC function. Gelatin methacrylate (GelMA) substrates with stiffnesses of 5 kPa (physiological) and 10 kPa (pathological) were seeded with human umbilical vein ECs (HUVECs) and subjected to constant physiological shear stress (12 dyne/cm²) for 6 hours. Analysis focused on heparan sulfate (HS), sialic acid (SA), hyaluronic acid (HA), syndecan-1 (SDC1), cluster of differentiation 44 (CD44), and Yes-associated protein (YAP). Compared to the 5 kPa conditions, HS coverage and thickness decreased at 10 kPa, indicating impaired barrier function and increased susceptibility to inflammatory agents. SA density increased despite decreased coverage, suggesting enhanced binding site availability for inflammatory recruitment. HA expression remained unchanged, but the amount of the HA core protein, CD44, was found to be increased at 10 kPa. Consistent with previously published interactions between CD44 and YAP, there was an increased YAP activation at 10 kPA, as evidenced by increased nuclear translocation and decreased phosphorylation. These findings, bridging biomaterials and mechanobiology approaches, deepen the understanding of how mechanical stimuli influence EC GCX function. The results underscore the potential of mechanotherapeutic strategies aimed at preserving vascular health by modulating endothelial function.

ABBREVIATIONS

AF: Alexa Flour
APS: Ammonium persulfate
BSA: Bovine serum albumin
CD44: Cluster of differentiation 44
DAPI: 4′,6-diamidino-2-phenylindole
ECs: Endothelial cells
EDTA: Ethylenediaminetetraacetic acid
eNOS: Endothelial nitric oxide synthase
GAG: Glycosaminoglycan
GCX: Glycocalyx
GelMA: Gelatin methacrylate
HA: Hyaluronic acid
HRP: Horseradish peroxidase
HS: Heparan Sulfate
HUVECS: Human umbilical vein
endothelial cells
ICAM-1: Intercellular adhesion
molecule 1
IgG: Immunoglobulin G
IgM: Immunoglobulin M
MAH: Methacrylic anhydride
MFI: Mean fluorescence intensity
PBS: Phosphate buffered saline
PECAM: Platelet endothelial cell
adhesion molecule
PMSF: Phenylmethylsulphonyl fluoride
SA: Sialic acid
SDC1: Syndecan-1
SDS: Sodium dodecyl sulfate
SEM: Standard error of the mean
TEMED: Tetramethylethylenediamine
VCAM-1: Vascular adhesion molecule 1
WGA: Wheat germ agglutinin
YAP: Yes-associated protein

Atherosclerosis, a precursor to cardiovascular diseases, accounts for 37% of deaths in individuals under 70 years old worldwide [1]. Severe atherosclerosis is often associated with increased blood vessel stiffness, which results from aging and systolic hypertension [2]. This disease begins with endothelial activation and progresses through a cascade of events, including vessel wall thickening, hardening, and narrowing, ultimately leading to flow obstruction [3].

The endothelium, lining the vessel luminal surface, is crucial for vascular health, regulating numerous cardiovascular functions such as vascular tone, hemostasis, permeability, and mechanotransduction. Endothelial cells (ECs) convert mechanical stimuli into biochemical or electrical signals that induce cellular responses. Research indicates that EC dysfunction is a primary cause of atherosclerosis [4]. Disruptions in the mechanical forces applied to ECs, including increased fluid shear stress from blood flow and tissue rigidity, can contribute to EC dysfunction, a major cause of atherosclerosis [5].

EC function and mechanotransduction heavily depend on the glycocalyx (GCX), a layer on the EC surface. Composed primarily of glycosaminoglycan (GAG) chains attached to core proteins, the GCX forms glycoproteins and proteoglycans essential for EC integrity [6,7]. It acts as a mechanotransducer, sensing fluid shear stress and modulating the activation of endothelial nitric oxide (NO) synthase (eNOS), as well as regulating inflammatory genes and adhesion molecule expression [8-10]. Previous studies conducted in static (no flow) conditions also highlight the influence of matrix stiffness on GCX composition and function, with elevated stiffness potentially exacerbating EC dysfunction [11,12]. The previous work indicates to explore how simultaneous stimulation of ECs by both shear stress and matrix stiffness impacts GCX-mediated mechanotransduction pathways, further building on the prior findings. By elucidating the complex interactions between the mechanical environment and the GCX, this work uncovers novel insights into the mechanisms underlying atherosclerosis initiation and progression [1-5]. Such insights could lead to new therapeutic strategies aimed at preserving GCX integrity and optimizing endothelial function, thereby mitigating cardiovascular risk, and improving patient outcomes.

Current approaches for modeling the effects of fluid shear stress and matrix stiffness on GCX expression include both in vivo animal models and in vitro cellular models. In vivo models involve surgical modifications of vascular structures to induce specific shear stress levels and acute in vitro models address certain limitations of animal models by using human cell cultures to investigate EC mechanoresponses under both physiological and pathological conditions. One approach involves culturing EC monolayers on cell culture plates placed on an orbital shaker to consistently apply fluid shear stress, although shear stress distribution can vary across the plate [16]. Another method utilizes a rotating cone within a cell culture plate to generate flow and shear stress on EC monolayers, minimizing shear stress gradients [17,18]. The parallel plate flow chamber method allows controlled fluid flow over EC monolayers, enabling exposure of EC monolayers to flow disturbances and shear stress gradients alongside regions of uniform flow and steady shear stress conditions [19-21]. However, ECs are often cultured on rigid surfaces that do not accurately mimic physiological strain and stiffness. To address this limitation, researchers have developed photolithography-fabricated devices using synthetic biomaterials, including cubic or cylindrical microfluidic channels coated with extracellular matrix proteins and ECs. However, these biomaterials may interfere with cell-matrix interactions [22-24].

This study addresses these challenges by introducing an in vitro flow chamber system designed to investigate the concurrent impacts of varying EC substrate stiffness and fluid shear stress on EC function, with a specific focus on the GCX. Gelatin methacrylate (GelMA) was selected as the matrix due to its high biocompatibility, non-swelling properties, tunable mechanical pathological blood flow patterns, allowing the study of how disturbances in local hemodynamics impact EC function and disease progression [13]. Additionally, animal models of hypertension are used to explore the effects of increased vessel wall stiffness on EC dysfunction [14]. However, variations in gene families and expression levels between animals and humans limit the translatability of findings from animal studies to human atherosclerotic conditions [15].

In vitro models address certain limitations of animal models by using human cell cultures to investigate EC mechanoresponses under both physiological and pathological conditions. One approach involves culturing EC monolayers on cell culture plates placed on an orbital shaker to consistently apply fluid shear stress, although shear stress distribution can vary across the plate [16]. Another method utilizes a rotating cone within a cell culture plate to generate flow and shear stress on EC monolayers, minimizing shear stress gradients [17,18]. The parallel plate flow chamber method allows controlled fluid flow over EC monolayers, enabling exposure of EC monolayers to flow disturbances and shear stress gradients alongside regions of uniform flow and steady shear stress conditions [19-21]. However, ECs are often cultured on rigid surfaces that do not accurately mimic physiological strain and stiffness. To address this limitation, researchers have developed photolithography-fabricated devices using synthetic biomaterials, including cubic or cylindrical microfluidic channels coated with extracellular matrix proteins and ECs. However, these biomaterials may interfere with cell-matrix interactions [22-24].

This study addresses these challenges by introducing an in vitro flow chamber system designed to investigate the concurrent impacts of varying EC substrate stiffness and fluid shear stress on EC function, with a specific focus on the GCX. Gelatin methacrylate (GelMA) was selected as the matrix due to its high biocompatibility, non-swelling properties, tunable mechanical characteristics, and the presence of adhesive molecules, facilitating straightforward and cost-effective hydrogel fabrication [12,25]. Hydrogels with stiffness levels of 5 kPa and 10 kPa, representing physiological and pathological conditions, were seeded with human umbilical vein ECs (HUVECs) to form monolayers. These hydrogels were integrated into a flow chamber device where cell culture media applied fluid shear stress to the HUVEC monolayer over a 6-hour period.

This model effectively combines non-swelling biomaterial components with a parallel-plate flow chamber setup to investigate how variations in substrate stiffness, while maintaining constant fluid shear stress, influence EC function. This approach lays the foundation for future work in which fluid shear stress will also be varied to mimic pathological disturbed flow and shear stress gradient conditions. The study explored the combined effects of constant fluid shear stress and varied substrate stiffness on HUVECs, focusing on mechanoresponsive molecules such as Yes-associated protein (YAP) and GCX components including heparan sulfate (HS), sialic acid (SA), hyaluronic acid (HA), syndecan-1 (SDC1), and cluster of differentiation 44 (CD44). The findings enhance the understanding of how mechanical stimuli affect EC GCX function and highlight the potential for mechanotherapeutic strategies aimed at maintaining vascular health by modulating endothelial GCX function. Additionally, the study underscores the importance of integrating biomaterials with mechanobiology approaches, demonstrating their critical role in advancing the knowledge of EC physiology.

### Methods

This model features a mechanically tunable substrate made from GelMA hydrogel, designed to emulate the stiffness of vessel walls under both physiological and pathological conditions. Additionally, the model includes a fluidic device that generates laminar flow, simulating the luminal mechanical environment by applying appropriate shear stress. This section describes the protocols for synthesizing GelMA, fabricating hydrogels with varying stiffness levels, designing the fluidic device, outlining experimental conditions, and data collection methods.

### Materials

Gelatin type A (300 bloom) from porcine skin, methacrylic anhydride (MAH), (trimethoxysilyl)propyl methacrylate, HA binding protein (HAP, 385911), goat serum, sodium chloride, Triton X-100, sodium dodecyl sulfate (SDS), ethylenediaminetetraacetic acid (EDTA), phenylmethylsulphonyl fluoride (PMSF), and sodium deoxycholate were obtained from Sigma-Aldrich (St. Louis, MO). Tetramethylethylenediamine (TEMED), ammonium persulfate (APS), acetone and glacial acetic acid, phosphate buffered saline (PBS), bovine serum albumin (BSA), paraformaldehyde, Alexa Flour (AF) 488-conjugated goat anti-mouse IgM (SA5-10294), phospho-YAP (PA5-17481), YAP1 (SU33-06, MA5-32117), beta actin loading control (BA3R, MA5-15739), horseradish peroxidase (HRP)-conjugated goat anti-rabbit IgG (G21234), and HRP-conjugated goat anti mouse IgG (31430) were purchased from Thermo Fisher Scientific (Waltham, MA). HUVECs, vascular basal medium, and EC growth kit were sourced from the American Type Culture Collection (ATCC, Manassas, VA). Cell culture transwells (353095) were purchased from Corning (Corning, NY). Biotinylated wheat germ agglutinin (WGA) lectin (PL-1025), biotinylated elderberry bark lectin (B-1305), and 4′, 6-diamidino-2-phenylindole (DAPI)-containing mounting media were procured from Vector Laboratories (Burlingame, CA). AF488-conjugated streptavidin (AB_2337249) was purchased from Jackson ImmunoResearch Inc. (West Grove, PA). The HS antibody (F58-10EA) was purchased from Amsbio LLC (Cambridge, MA). AF555 conjugated Phalloidin (8953S) was purchased from Cell Signaling Technology (Danvers, MA). Protease inhibitor was purchased from Roche (Mannheim, Germany). Electrophoresis and Western blot supplements including 30% polyacrylamide/Bis solution, 10× Tris/Glycine/SDS buffer, Laemmli Sample Buffer, and enhanced chemiluminescence (ECL) were purchased from Bio-Rad. Antibody against syndecan-1 (A-6, sc-390791), mouse monoclonal IgG PECAM-1 antibody (sc-376764), and polybrene (10 mg/mL; SC134220) were purchased from Santa Cruz Biotechnology (Dallas, TX). AF488-conjugated goat anti-mouse IgG (H+L) antibody was purchased from Invitrogen (Carlsbad, California). Lastly, CD44 shRNA lentiviral particles (TL314080A, TL314080C) were purchased from Origene (Rockville, MD).

### Substrate Fabrication

Synthesis of GelMA: The synthesis of GelMA, used to fabricate hydrogels with non-swelling properties, was developed and characterized in previous work [12]. The same protocol was followed for this study. Initially, porcine skin type A, 300-bloom gelatin was dissolved in a 0.25M carbonate-bicarbonate buffer at a concentration of 10% (w/v). The pH was adjusted to 9.5, followed by the addition of methacrylic anhydride (MAH) at a 1:100 volume ratio to start the methacrylation reaction. Subsequently, the GelMA solution 551 was precipitated using acetone, dried overnight in a vacuum oven at room temperature (FIG. 5), and stored at −20° C.

Fabrication of Gelatin-coated GelMA Substrate: Microscope slides were employed as a platform for hydrogel attachment to enhance the handling and stability of the hydrogels under flow conditions. As previously reported [12], the microscope slides were treated with 0.5% 3-(Trimethoxysilyl) propyl methacrylate in absolute ethanol and 3% glacial acetic acid. The reaction was allowed to proceed for 5 minutes, after which the slides were washed with ethanol, air-dried, and stored in the dark at room temperature for future use. For hydrogels mimicking physiological stiffness (5 kPa), 4% (w/V) GelMA was dissolved in DI water and polymerized through redox-induced free radical polymerization by adding 24 mM TEMED and 24 mM APS. Conversely, to represent pathological stiffness (10 kPa), an 8% (w/V) GelMA solution was prepared and polymerized using 6.25 mM TEMED and 24.5 mM APS [12]. The polymer solution was added to molds to form the substrates (FIG. 5). The substrates were coated with a 60 μg/mL gelatin solution for 45 minutes at 37° C. before cell seeding to enhance initial cell adhesion.

### Flow Chamber Design

A custom-designed parallel-plates flow chamber was used to replicate the luminal mechanical environment of a physiological artery (FIGS. 1-4). This device was designed using SOLIDWORKS software to produce uniform laminar flow with a shear stress of 12 dyne/cm2. Additionally, the flow chamber was engineered to accommodate hydrogels of various stiffness levels, enhancing the precision of the in vitro model. The Flow Simulation feature in SOLIDWORKS was used to analyze flow conditions and determine the appropriate flow rate required to achieve a shear stress of 12 dyne/cm² on the apical surface of the endothelial monolayer on the hydrogels [26].

### Endothelial Cell Culture

HUVECs were selected as the primary human ECs for this study to investigate the impact of the mechanical environment on GCX expression. The extensive existing data on this cell type facilitated the evaluation of these findings. The cells were cultured in vascular cell basal medium enriched with various growth factors and supplements: recombinant human vascular endothelial growth factor (rh-VEGF: 5 ng/mL), epidermal growth factor (rh-EGF: 5 ng/mL), basic fibroblast growth factor (rh-FGF basic: 5 ng/mL), insulin-like growth factor-1 (rh-IGF-1:15 ng/mL), L-glutamine (10 mM), heparan sulfate (0.75 Units/mL), hydrocortisone (1 μg/mL), ascorbic acid (50 μg/mL), fetal bovine serum (2% FBS), and 1% penicillin/streptomycin. HUVECs between passages 4 and 7 were seeded at a density of 50,000 cells/cm² on the 5 kPa and 10 kPa hydrogel substrates. While the EC were growing on the hydrogels, their culture medium was changed every two days until a complete endothelial monolayer was formed.

### Running Uniform Flow

After reaching 100% confluency, the HUVEC-seeded hydrogels were placed in the flow chamber 554a to apply a shear stress 554b of 12 dyne/cm² for six hours 555 (FIG. 5). Initially, the substrate and sterilized components of the flow chamber were assembled within a biosafety cabinet. Medium supplemented with 0.5% BSA was added to the flow chamber, which was then transferred to a 37° C., 5% CO2, humidified incubator. ECs were gradually acclimated to the shear stress by incrementally increasing the flow rate until 12 dyne/cm² was achieved. After maintaining this condition for six hours, the flow was stopped, and the samples were prepared for subsequent procedures.

### Histological Analysis

To investigate the response of ECs to various mechanical conditions, cells that underwent the six-hour flow experiment were washed with PBS containing 1% BSA and fixed in a 4% paraformaldehyde solution for fifteen minutes at room temperature. Initially, cytoskeleton reorganization in response to mechanical stimulation was examined. The ECs were permeabilized with 0.1% Triton X-100 for three minutes and then blocked with 3% BSA. The samples were then incubated for one hour with AF555-conjugated Phalloidin at a 1:100 dilution for F-actin visualization.

After examining cytoskeleton reorganization, YAP translocation in response to mechanical stimulation was investigated. The paraformaldehyde-fixed ECs were subjected to permeabilization with 0.1% Triton X-100 for fifteen minutes and then blockage with 5% goat serum for one hour. This was followed by EC incubation with anti-YAP 1 antibody at a 1:200 dilution for forty-eight hours at 4° C. Next, the samples were incubated with AF488-conjugated goat anti-rabbit IgG at a 1:1000 dilution for one hour for secondary labeling. YAP expression is subtle in certain conditions, warranting the performance of additional control studies to confirm the integrity of the YAP immunocytochemical staining protocol. For the control studies, ECs were cultured on 0.4 m pore sized Transwell inserts (Falcon®) for expeditious experimentation. Various confluency levels were examined, since YAP expression has been suggested to be dependent on confluence level. The ECs were exposed to either static or flow conditions to maintain consistency with previously published work, allowing for a direct comparison of these results with established YAP findings. For the flow-conditioned cohort of control EC samples, the EC monolayers were transferred on the Transwell inserts (Falcon®) to a flow device that is compatible with the inserts, as previously described [27].

After examining cytoskeleton reorganization and YAP translocation in response to mechanical stimulation, expression of GCX as a whole while also narrowing in on individual components of the GCX was investigated. The paraformaldehyde-fixed ECs were subjected to 3% BSA blocking solution for one hour. Then, biotinylated WGA was used to label whole GCX, and biotinylated elderberry bark lectin was used for SA labeling, both at a 1:100 dilution. These lectins were incubated with the ECs for one hour at room temperature. AF488-conjugated streptavidin at a 1:1000 dilution was used for secondary labeling for one hour at room temperature. For the HS and HA components of the GCX, samples were placed in humid chambers and incubated with F58-10E4 anti-HS antibody or biotinylated HA binding protein at a 1:100 dilution for three days at 4° C. This step was followed by secondary labeling with AF488-conjugated goat anti-mouse IgM for HS or AF488-conjugated streptavidin for HA at dilutions of 1:400 and 1:50, respectively, for one hour. It is important to acknowledge that GCX expression on the EC membrane was not validated or in junctional areas with markers such as VE-cadherin, which could be perceived as a potential limitation of this approach. However, ECs were successfully co-stained for GCX and cell-cell junction proteins in the past [27]. Given that this study is testing a new model, co-staining was not implemented to avoid potential interference between dual labeling agents, which can sometimes disrupt GCX signal. Additionally, the hydrogel itself may introduce interference.

To investigate EC expression of adhesion molecules, platelet endothelial cell adhesion molecule (PECAM) was assessed. After fixing the cells in 4% paraformaldehyde and blocking with 3% BSA in PBS, they were incubated overnight with a 1:100 dilution of mouse PECAM-1 monoclonal antibody. The sample was then visualized following a 1-hour incubation with a 1:200 dilution of AF488-conjugated goat anti-mouse IgG (H+L) secondary antibody.

It is important to note that labeling of cytoskeleton, YAP, GCX components, and PECAM-1 was performed on subsamples. Specifically, two regions (subsamples) were cut from the HUVEC monolayer samples on hydrogels prior to labeling. After histological staining, as described above, the subsamples were mounted on glass slides with DAPI-containing mounting media. Subsequently, confocal imaging was performed. Zeiss LSM 800 and 710 confocal microscopes were used to capture z-stack images at 63× magnification from three random areas of each subsample, capturing three images per subsample.

### Image Analysis

First, confocal microscope images of EC monolayers in which actin was stained with fluorescent phalloidin were visually assessed for actin filament changes in organization and distribution across experimental conditions. The analysis focused on qualitative features, such as filament density, morphology, and the presence of actin stress fibers or networks. The data were qualitatively compared between experimental groups, and representative images were selected to illustrate findings. No quantitative analysis or intensity measurements were performed.

This study addressed the challenge of analyzing YAP subcellular localization with high resolution, given the subtlety of its fluorescent signal, by using a custom-designed Python script, publicly available here: https://github.com/Cheryl73/Image_code_YuChen. The script was developed to process immunofluorescent images of EC monolayers and quantify changes in YAP localization within individual ECs. First, the Python program removed noise from the images and detected the YAP signal across each layer of the 3D stack, recording the pixel coordinates (x, y, z). The same process was applied to DAPI, which labels the nucleus. Pixels that were positive for both YAP and DAPI were classified as nuclear YAP, while those positive only for YAP were categorized as cytosolic YAP. Note that the Python script collected continuous pixel data per YAP image. The nuclear-to-cytosolic YAP ratio was then calculated by dividing the count of nuclear YAP pixels by cytosolic YAP pixels.

The custom Python program (https://github.com/Cheryl73/Image_code_YuChen) was used to analyze GCX and its components; as stated above, the Python program collected continuous data per GCX image. The GCX thickness, which ranges from a few micrometers to several, falls within the resolution capabilities of confocal microscopy. Specifically, the Z-axis resolution of confocal imaging (typically 200-300 nm) is sufficient to measure GCX thickness. However, there were concerned that autofluorescence from the hydrogel might interfere with the GCX signal. To address this, the Python program applied the Otsu method to reduce noise by subtracting negative control signals. For component thickness measurement, image stacks were used to generate 3D reconstructions, and the thickness (Δz) of the targeted components was measured at each pixel in the X-Y plane. Signals above the nucleus were classified as apical, while signals below the nucleus were considered basal. In regions where the microscope's resolution was insufficient or in continuous layers such as cell-cell junctions, where apical and basal signals could not be distinguished, thickness was recorded as a single value. Thickness measurements were normalized to the 5 kPa condition. This approach for resolving Z-axis GCX thickness on hydrogels using Python-based quantitative analysis has been fully validated, as outlined in a recent publication [26]. In addition to thickness, the expression of GCX and its components was quantified by averaging the fluorescence intensity at each pixel, reported as mean fluorescence intensity (MFI). The percentage of the endothelial surface area covered by the GCX and its components was calculated by dividing the number of pixels with expression by the total number of pixels in the en face view.

### Western Blot

Western blot analysis was conducted to evaluate the dysregulation of the SDC1 and CD44 core proteins of the GCX, as well as the dephosphorylation of YAP in response to stimulation by a stiff substrate as opposed to a soft substrate. After the 6-hour flow experiment, cells were washed with cold PBS and detached using a scraper into an ice-cold nonenzymatic dissociation reagent solution. The cells were then collected by centrifugation at 450×g. Cell lysis was performed using radio-immunoprecipitation assay (RIPA) buffer (150 mM sodium chloride, 1% Triton X-100, 0.5% sodium deoxycholate, 0.1% SDS, and 50 mM Tris) supplemented with 1 mM PMSF, 5 mM EDTA, 1 mM sodium orthovanadate, and 1× protein inhibitor. A total of 15 g of protein from each sample was separated by sodium dodecyl sulfate-polyacrylamide gel electrophoresis (SDS-PAGE) and then transferred onto a polyvinylidene difluoride (PVDF) membrane. The blots were blocked using 5% non-fat milk in Tris buffered solution with 0.1% Tween 20 (TBST) for 1 hour at room temperature, then incubated overnight at 4° C. with the following primary antibodies: SDC1 (1:700 dilution), CD44 (1:3000 dilution), YAP (1:1000 dilution), and phospho-YAP (1:1000 dilution). Beta-actin (1:3000 dilution) served as the loading control. Detection was achieved using secondary antibodies, including HRP-conjugated goat anti-rabbit and HRP-conjugated goat anti-mouse IgG (1:5000), followed by chemiluminescence reagents and imaging, and densitometry measurements. Densitometry measurements were used to quantify the intensity of the Western blot protein bands and evaluate the relative abundance of SDC1, CD44, YAP and phospho-YAP across experimental conditions.

Lentiviral shRNA-Mediated Knockdown of CD44 in HUVECs

HUVECs were transduced with shRNA-containing lentiviral particles targeting CD44. To accomplish this, the cells were first seeded at a density of 1×10⁵ cells per well in 6-well plates and incubated overnight at 37° C. with 5% CO₂ to allow for attachment. Two different shRNA-containing lentiviral particles were tested to optimize transient knockdown efficiency. Lentiviral particles were diluted to achieve a multiplicity of infection of 10 and supplemented with polybrene (10 μg/mL) to enhance transduction efficiency. The culture medium was replaced with 1 mL of the lentiviral mix, and plates were gently swirled to ensure even distribution. Cells were incubated for 24 hours at 37° C. with 5% CO₂ to allow for viral transduction. Following transduction, the lentiviral mix was removed, and cells were gently washed with PBS to eliminate residual virus. Fresh culture media was added, and cells were allowed to grow to full confluency before subsequent lysis and Western blot analysis of CD44, YAP, and phospho-YAP.

### Statistical Analysis

All data are reported as mean±standard error of the mean (SEM). Statistical analyses were conducted using Minitab and GraphPad Prism software. The normality of data distribution was confirmed using the Shapiro-Wilk method, except in cases where most data points had identical values. In such cases, the D'Agostino-Pearson test was utilized. Student's t-test was subsequently used to assess statistical significance (α=0.05). In the figures, the significance levels and corresponding p-values are indicated as follows: non-significant (ns), *p<0.05, **p<0.01, ***p<0.001, and ****p<0.0001.

### Results

To compare GCX expression under physiological and pathological conditions, HUVECs were cultured on hydrogels with stiffnesses of 5 kPa and 10 kPa, respectively, and exposed to a shear stress of 12 dyne/cm² for six hours. Computational fluid dynamics analysis 554b was conducted to evaluate flow conditions when substrates were inserted into fluidic devices (FIG. 5). This analysis demonstrated that ECs experienced uniform shear stress 554b (FIG. 5), except at the edges of the hydrogels, where flow disturbances often occurred due to the transition between the flow chamber and the hydrogel surface (FIG. 5). Informed by these results, the ECs near the edges of the hydrogels were excluded from the data extraction process.

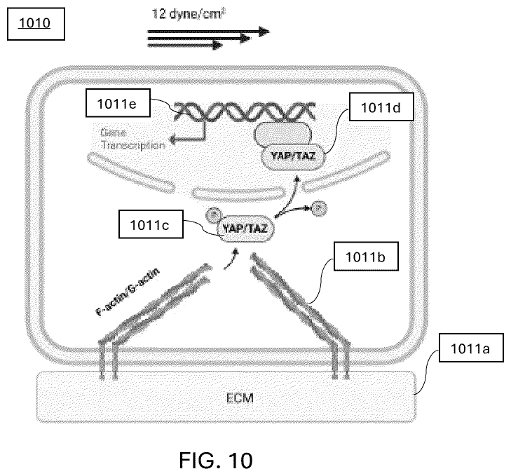

To assess the device's performance and accuracy in mimicking mechanical environments, cytoskeleton reorganization alongside YAP activation and translocation 1010 was evaluated. Actin reorganization 1011b through polymerization is known to induce YAP dephosphorylation, activation, and subsequent nuclear translocation 1011c and 1011d (FIG. 10) [28], making these markers indicators of the device's effectiveness. F-actin reorganization and YAP-1 expression in HUVECs 1011e on 5 kPa and 10 kPa substrates 1011a exposed to 12 dyne/cm² shear stress.

Under healthy conditions, actin fibers were more peripheral, while stress fibers were distributed across the entire cell body in diseased conditions. On 5 kPa hydrogels, actin filaments were mainly concentrated in the peripheral regions of the cells, with distinct cortical actin structures visible along the cell edges, outlining the cell membrane. No prominent actin stress fibers were observed in the internal compartments of the ECs. In contrast, on 10 kPa hydrogels, cells displayed more pronounced stress fibers spanning the entire cell body, indicating a shift toward a pathological state. These stress fibers, which are associated with increased cellular contractility and mechanical tension, were more abundant and thicker in the central regions of the cell, reflecting changes in cytoskeletal organization. These results, showing that matrix stiffness influences actin organization in a way that mirrors both physiological and pathological conditions, confirm the device's performance and accuracy.

YAP translocation was quantified by calculating the ratio of nuclear YAP pixels to cytosolic YAP pixels. Quantitative analysis showed that YAP translocation from the cytosol to the nucleus increased significantly under pathological conditions compared to physiological conditions (p<0.05). YAP translocation to the nucleus occurs when YAP is activated, leading to proinflammatory gene expression. To further investigate YAP activation, as indicated by dephosphorylation, Western blot analysis was performed. The results revealed a significant decrease in the ratio of phospho-YAP to total YAP at 10 kPa compared to 5 kPa (p<0.05). This Western blot data supports the YAP translocation findings obtained from immunofluorescent microscopy images. Therefore, it can be concluded that on the stiffer substrate, more YAP was activated via dephosphorylation, enabling YAP to move from the cytosolic to the nuclear compartment of the ECs.

Building on this, YAP localization was evaluated as a downstream indicator of mechanotransduction activity. Qualitative immunocytochemical analysis revealed that YAP was predominantly localized in the cytoplasm at moderate expression levels, with very low nuclear presence, which is indicative of a quiescent endothelial cell (EC) state. Although nuclear YAP levels remained low overall, a subtle but noticeable increase in nuclear YAP was observed under 10 kPa conditions compared to 5 kPa. This shift suggests an activation of YAP signaling in response to increased matrix stiffness, which is commonly associated with a more active, potentially pathological cellular state. These findings highlight the role of matrix stiffness in modulating YAP localization, providing insight into its involvement in cellular responses to mechanical cues.

To authenticate the qualitative YAP staining results, localization was examined in both positive and negative control ECs. Positive controls were defined as subconfluent and/or static conditions, while negative controls were confluent and/or exposed to flow conditions. Under static conditions at subconfluency, YAP was predominantly localized in the nucleus, indicating a proliferative state. In contrast, uniform flow (12 dyne/cm²) at subconfluency resulted in a diffuse distribution of YAP between the cytoplasm and nucleus, reflecting flow-induced mechanotransduction. At confluency, YAP was primarily localized in the cytoplasm under both static and flow conditions. However, under flow conditions at confluency, while most cells did not exhibit nuclear YAP expression, some cells displayed distinct YAP puncta, suggesting localized mechanotransduction or protein aggregation. As noted earlier, all control data were collected from ECs grown on Transwell inserts rather than hydrogels, for simplicity. These results confirm the specificity and reproducibility of the YAP staining results.

Quantitative analysis of YAP localization was essential, as nuclear expression and translocation of YAP are difficult to resolve with the naked eye in ECs on hydrogels and under flow conditions. Using a custom-designed Python script, YAP was probed at high resolution, revealing a significant increase in nuclear YAP localization on 10 kPa hydrogels compared to 5 kPa (p<0.05). The ratio of nuclear to cytosolic YAP was determined by comparing the number of YAP-positive pixels in the nucleus and cytoplasm. These results indicate a shift toward increased nuclear YAP on the stiffer 10 kPa substrates, suggesting enhanced mechanotransduction and a transition to a more active cellular state. Western blot analysis corroborated the Python analysis of fluorescently labeled ECs, showing a decreased phospho-YAP to total YAP ratio on stiffer hydrogels (p<0.05). This indicates increased YAP activation via dephosphorylation on the pathological substrate. Together, the quantitative analysis of the fluorescent images and Western blots confirm that ECs respond distinctly to substrate stiffness through YAP activation, further validating the device's performance and accuracy.

Once the device's performance was validated, it was employed in GCX remodeling studies. The substrate stiffness does not notably affect the overall expression of GCX (from en face views of the endothelium) or its thickness (from orthogonal views) in HUVECs under uniform shear stress. Quantitative analysis indicates no significant difference in normalized GCX expression, as assessed by comparing MFI or GCX thickness between the two substrate stiffness conditions. These findings are consistent with previous study, where the impact of 2.5 kPa, 5 kPa, and 10 kPa on GCX structure in ECs under static conditions were studied. It was found that overall GCX expression is not dysregulated in response to increased substrate stiffness [12].

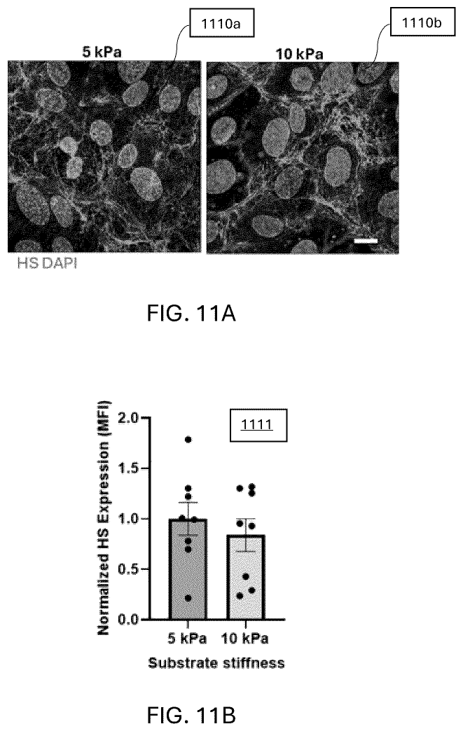

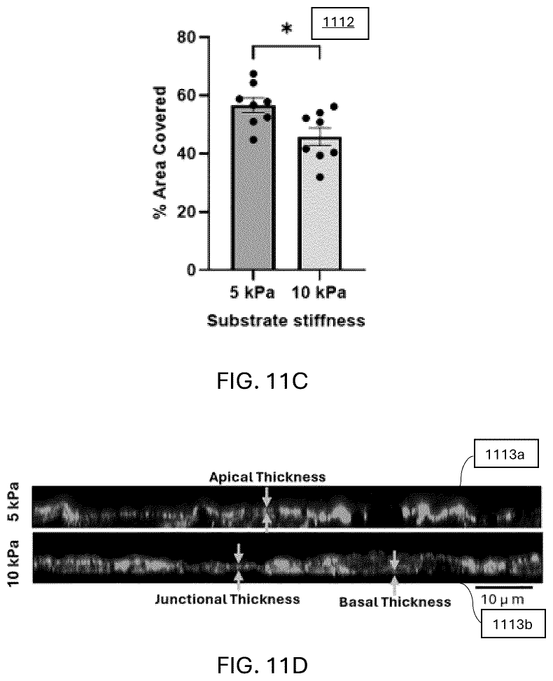

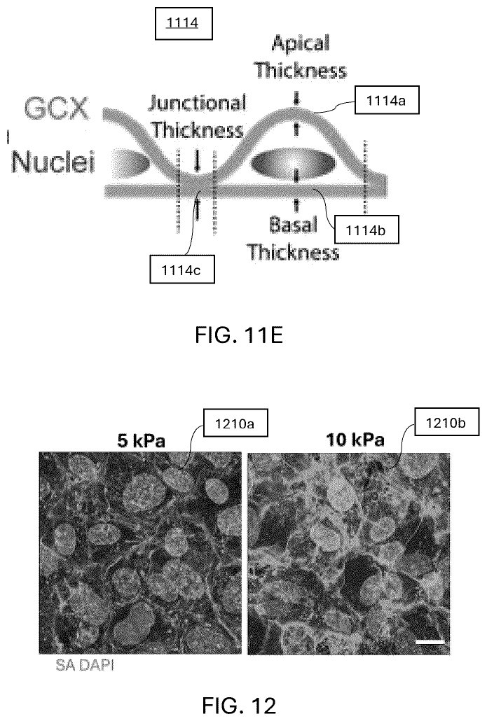

Although the overall GCX does not appear to be affected by substrate stiffness, individual components of the GCX exhibit responsiveness, with the nature of this response varying based on the specific GCX component type. FIG. 11A illustrates the effect of substrate stiffness on HS expression, distribution, and thickness in ECs. When substrate stiffness changed in uniform flow conditions, there was no change in HS expression as measured by intensity of the fluorescent signal 1110a and 1110b (FIG. 11B), although there was a significant decrease in the percentage area covered by HS 1111 at 10 kPa compared to 5 kPa 1112 (p<0.05) (FIG. 11C). In contrast, previous studies under static conditions demonstrated a significant decrease in HS expression in HUVECs with increasing stiffness, along with reduced area coverage [12,29]. Orthogonal views illustrate HS thickness in HUVEC monolayers (FIG. 11D). Based on the location of the expressed HS as seen in FIG. 11D, the thickness of HS 1113a and 1113b was categorized into junctional 1114c, apical 1114a, and basal 1114b compartments 1114 (FIG. 11E). This analysis indicated a significant reduction of HS thickness at the cell junction in 10 kPa compared to 5 kPa conditions (p<0.05). However, the thickness measured at the apex and the base of the HUVECs did not show a significant change.

Syndecan-1 is a transmembrane receptor that provides extracellular binding sites for HS and connects to the intracellular cytoskeleton, anchoring the GCX to the ECs in a transmembrane manner [30]. Therefore, a Western blot analysis was conducted to investigate the dysregulation of SDC1 in response to increased substrate stiffness under uniform flow conditions with 12 dyne/cm² shear stress. No significant difference was found in normalized SDC1 levels between the two substrate stiffness conditions.

FIG. 12 shows that substrate stiffness significantly affects SA expression and thickness in HUVECs. A significant increase in SA expression was observed in cells cultured on 10 kPa hydrogels 1210b compared to 5 kPa hydrogels 1210a (p<0.01) (FIG. 12). This suggests a higher density and greater aggregation of SA. Under uniform flow conditions, the area covered by SA is significantly lower on 10 kPa substrates compared to 5 kPa substrates (p<0.001). At cell junctions, SA thickness decreased by increasing substrate stiffness from 5 kPa to 10 kPa (p<0.001), while SA thickness at the apex and base of the cells did not show a significant change.

HA MFI/expression, percent coverage of ECs, and thickness were not altered in response to increased substrate stiffness under 12 dyne/cm² shear stress. However, the previous study showed that HA thickness decreased with increased substrate stiffness to pathological levels under static conditions [12]. Therefore, HA was investigated further by examining CD44 expression, a glycoprotein on the EC surface that binds to HA. Unlike the stable levels of HA, it was found that CD44 expression significantly increased at 10 kPa compared to 5 kPa (p<0.05). This increase in CD44 likely enhances HA stability on the endothelium.

### Discussion

To enhance the accuracy of in vitro models, a more practical and relevant approach is needed. In this regard, a device was developed that integrates non-swelling hydrogels with varying stiffnesses into a milli-fluidic chamber, designed to replicate both physiological and pathological conditions while subjecting cells to laminar flow [26]. This device can apply different laminar flows to hydrogels of varying stiffness. For this study, a constant flow-derived shear stress of 12 dyne/cm² was selected to simulate the physiological shear stress experienced by ECs in human arteries [31,32], while substrate stiffnesses of 5 kPa and 10 kPa were chosen to represent healthy and diseased conditions, respectively. The objective of this study was to perform foundational experiments that will lead to future studies exploring the effects of varying fluid shear stress to simulate disturbed flow and shear stress gradients, superimposed on both healthy and diseased substrates. It is anticipated that these studies will aid in optimizing the model for investigating hypertension-related atherosclerosis.

Under 12 dyne/cm² shear stress and 5 kPa versus 10 kPa substrate stiffness conditions, cytoskeletal organization was first examined as an indicator of EC response to mechanical cues. The actin cytoskeleton, a critical regulator of EC mechanobiology, exhibited stiffness-dependent remodeling under flow conditions. On 5 kPa hydrogels, peripheral actin fibers were prominent, consistent with a physiological state characterized by intact barrier integrity and lower contractile tension [33-35]. In contrast, stress fibers spanning the cell body were dominant on 10 kPa hydrogels, indicative of pathological conditions with increased cytoskeletal tension and mechanosensitivity. This finding aligns with previous studies showing that stiffer substrates promote stress fiber formation [36-40]. It is important to note that cytoskeletal responses to flow begin within an hour of exposure, although full reorganization typically requires more time [40-42]. Given that this study only exposed ECs to flow for six hours, this duration likely accounts for the absence of flow-induced EC elongation and alignment along the flow direction, which generally takes longer to manifest [40-42]. Nevertheless, since the peripheral actin network plays a role in various EC functions, such as cell-cell adhesion and maintaining barrier integrity [43], the increased presence of this network in the healthy experimental group, compared to the pathological group with extensive stress fibers, supports the device's ability to model both healthy and diseased conditions effectively.

Actin stress fibers, which are prominent on stiff substrates, facilitate YAP translocation to the nucleus [28,44]. To further validate the in vitro model, YAP translocation was examined, which was significantly influenced by substrate stiffness. On 5 kPa hydrogels, YAP localization to the nucleus was minimal compared to localization to the cytoplasm, while on 10 kPa hydrogels, there was an increase in nuclear YAP localization, although cytoplasmic YAP remained unchanged. Notably, nuclear-to-cytoplasmic YAP ratios were generally low in both healthy and diseased conditions, likely due to full confluency and mechanical stimulation [44]. Control EC samples conditioned at pre-confluency versus full confluency, as well as under static and flow conditions, support this, consistent with the well-established observation that YAP responds to mechanical forces like shear stress, confluency, and substrate stiffness, with nuclear localization occurring when cells are active and cytoplasmic localization when cells are quiescent [34,45]. Specifically, at subconfluency under static conditions, YAP was mainly nuclear, reflecting activation in the absence of contact inhibition [45]. Under flow at subconfluency, YAP was diffusely distributed, indicating flow-induced mechanotransduction [46,47]. At confluency, YAP was primarily cytoplasmic under both static and flow conditions, consistent with a quiescent state [45]. However, some cells under flow at confluency exhibited distinct YAP puncta, suggesting localized mechanotransduction or protein aggregation due to shear stress at high cell density [46,47]. This pattern highlights the complexity of YAP signaling in response to combined mechanical and confluency cues, reinforcing its role as a mechanosensor and validating the low nuclear-to-cytoplasmic YAP localization ratios observed in both 5 kPa and 10 kPa hydrogel conditions.

Actin stress fiber-induced YAP translocation to the nucleus is facilitated by YAP activation through dephosphorylation [28,44]. Western blot analysis revealed a significant decrease in the ratio of phospho-YAP to total YAP (p<0.05) in the pathological (10 kPa) group compared to the physiological (5 kPa) group, indicating increased YAP activation in the diseased state. This finding corroborates the observed cytoskeletal reorganization and increased nuclear YAP expression. Together, these results suggest that cells exposed to 10 kPa hydrogels under 12 dyne/cm² shear stress laminar flow adopt a more pro-inflammatory and dysfunctional endothelial phenotype, compared to cells exposed to 5 kPa hydrogels. These findings confirm the device's ability to model both healthy and diseased conditions, allowing for further investigation of GCX responses in these environments.

The GCX response to substrate stiffness changes under constant flow conditions were examined next. Staining results revealed that the intensity and thickness of WGA-stained GCX remained consistent across all substrate stiffness groups. This observation aligns with previous research, which showed that HUVECs under static conditions exhibited unchanged GCX expression despite variations in substrate stiffness [12]. Taken together, the present findings along with previous results suggest that substrate stiffness does not impact whole GCX density, endothelial coverage, or thickness. Instead, the GCX appears to be primarily influenced by the type of flow, as has been shown for ECs transitioning from uniform laminar to disturbed laminar flow [12,20,48]. Future studies include further investigating this phenomenon in future research, where uniform versus disturbed fluid shear stress will be superimposed on both soft and stiff substrates.

Unlike the behavior of the whole GCX, HS expression was influenced by substrate stiffness when ECs were exposed to physiological versus pathological conditions in this system. HS plays a critical role in various EC functions, including inhibiting immune cell adhesion, anticoagulation, mechanosensing, mechanotransduction, and vasodilation [6,49-51]. Previous studies have shown that HS expression, measured by fluorescence intensity, depends on substrate stiffness under static conditions [12,29]. However, these findings indicate that HS expression was unaffected by the combined application of shear stress and stiffness (FIGS. 11A and 11B). Despite this, a significant reduction in the endothelium area covered by HS on HUVECs cultured on 10 kPa substrates (FIG. 11E) was observed. This suggests that although HS expression levels remain unchanged, its distribution is altered, potentially reducing its barrier function. Since HS helps cover adhesion molecules like intercellular adhesion molecule 1 (ICAM-1) and vascular adhesion molecule 1 (VCAM-1) [52], a decrease in HS coverage could expose these molecules, making them more accessible to inflammatory cells and increasing adhesion to the EC surface. Furthermore, the HS layer at cell junctions was thicker in the physiological group, whereas it was thinner in the pathological group, indicating that the endothelium may be more susceptible to permeabilization under pathological conditions. Collectively, these findings suggest that altered HS distribution in pathological conditions may compromise the barrier function of the GCX.

Syndecan-1 is a HS proteoglycan that plays a crucial role in vascular homeostasis. HS covalently binds to at least one of the five binding sites on the core protein of SDC1 [53,54]. The number of GCX sugar chains (HS and others) attached to SDC1 can alter GCX function and impact cell-cell interactions [54]. Therefore, SDC1 expression was assessed under both physiological and pathological conditions, with no significant difference detected. These findings suggest that the observed differences in HS thickness and coverage area are not attributable to SDC1 loss. Instead, they may result from HS detachment from SDC1, leading to reduced coverage, and the cleavage of some HS chains, which decreases thickness at junctions.

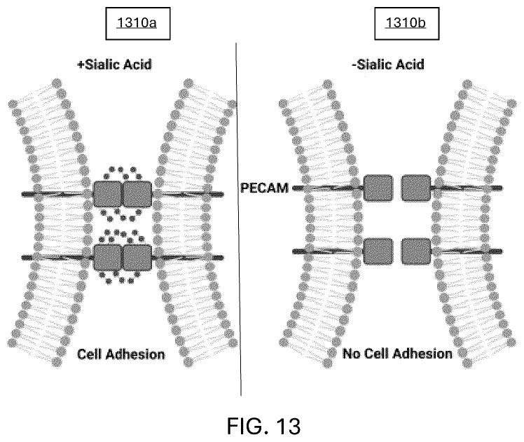

SA was the next sub-component of the GCX to be investigated. SA is an abundant component that, due to its negative charge, helps repel other cells and negatively charged substances [55]. However, some positively charged amino acids can bind to SA, providing binding sites that can be used by immune cells, potentially leading to inflammation [56,57]. SA expression was thus investigated under healthy and diseased conditions. These results showed that SA expression was upregulated under pathological conditions, while the area of coverage decreased. This suggests that more SA availability increases binding sites for immune cells, while the repulsive effect of SA decreases 1310b as EC surface coverage is reduced. α2-6 SA regulates the homophilic interaction of PECAM in ECs (FIG. 13) [58]. The reduced junctional thickness of SA 1310a in pathologically stimulated ECs may disrupt PECAM's ability to facilitate tight junction formation between ECs, potentially leading to increased permeability in pathological conditions. It is noteworthy that increased SA expression due to higher stiffness was previously observed under static conditions [12].

Example 3 assessed the feasibility that SA changes could be linked to barrier function in ECs, by probing for adhesion molecule expression in this context. While the overall amount of PECAM-1 expressed on the EC surface was the same for 5 and 10 kPa conditions, its coverage (distribution over ECs) and junctional thickness increased (15% increase in coverage and 28% increase in thickness) on 10 kPa compared to 5 kPa condition. PECAM-1 has been previously implicated as a pro-inflammatory marker through its role of mediating leukocyte transmigration at the intercellular junctions of endothelial cells [59-62] and has also been shown to contribute to atherosclerotic formation under disturbed flow conditions [63]. The PECAM-1 findings demonstrate that 10 kPa promotes a pro-inflammatory phenotype with an increase in PECAM-1 thickness at the junctions and coverage over ECs. Regarding functional assays, including nanoparticle uptake and permeability of circulating suspension cells, they are currently underway to more definitively link SA changes to barrier function in ECs cultured on surfaces with physiological versus pathological stiffnesses.

Unlike other GCX sugar chains, HA is not covalently bound to glycoproteins but is connected to the cell membrane via CD44 [64]. HA is essential for EC mechanosensing and dormancy [65]. Additionally, HA plays a role in the barrier function of ECs, and disruption in HA synthesis enhances immune cell adhesion to ECs, contributing to atherosclerosis [66]. No significant changes were observed in HA expression levels, EC surface area coverage, or thickness when comparing healthy conditions to diseased conditions (FIG. 12). This suggests that uniform laminar flow has a protective effect on HA. However, a previous study reported some decrease in HA thickness for ECs exposed to 10 kPa stiffness compared to 5 kPa stiffness under static conditions [12].

Although HA did not change in response to the mechanical environment in this study, its anchor point, CD44, exhibited altered expression under different mechanical conditions. Both in vivo and in vitro studies have shown that CD44 expression is upregulated in atheroprone areas where plaques and macrophages are more abundant, leading to increased immune cell adhesion and transmigration across the endothelium [67-69]. Additionally, CD44 enhances the secretion of proinflammatory cytokines such as IL 1β and IL-6 in ECs, which, in turn, upregulate CD44 expression, creating a positive feedback loop [70,71]. In this study, under laminar flow conditions, CD44 expression on 10 kPa substrates increased compared to 5 kPa substrates. This suggests that even under physiological laminar flow, a stiffer substrate can upregulate CD44. This in vitro finding, which did not involve blood components or immune cells, implies that increased CD44 expression may contribute to the recruitment of macrophages and the formation of atherosclerotic plaques.

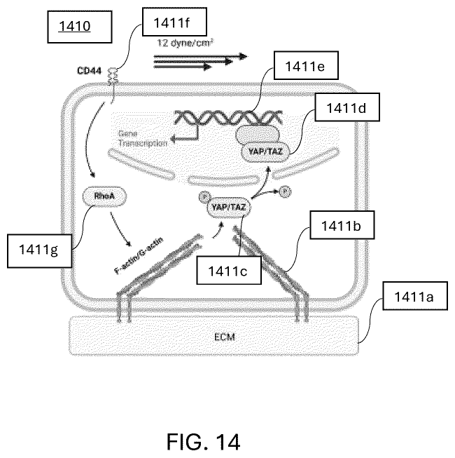

The CD44 findings prompt us to revisit the relationship between YAP and actin to connect the results. The interaction between specific CD44 variants and YAP has been demonstrated in cancer cells, where CD44 enhances YAP1/TEAD activation [72,73]. Knockdown of CD44 has been shown to reduce YAP expression and the levels of YAP's target inflammatory genes, such as CTGF and CYR61, while increasing phosphorylated YAP (s127) [73,74]. Stiffness-induced YAP translocation has been reported to occur via the CD44-RhoA-YAP signaling pathway in fibroblasts, with RhoA serving as a regulator of actin cytoskeleton dynamics [75]. Under diseased conditions 1410, increased CD44 expression 1411f, actin stress fiber formation 1411b, and heightened YAP 1411c activation suggest that CD44 senses the mechanical environment, transmitting signals through RhoA 1411g and the cytoskeleton to YAP. YAP then translocates to the nucleus 1411d to induce gene expression 1411e changes that drive biological responses. This implies that the GCX 1411a could be an upstream effector of YAP via CD44 (FIG. 14). To test this, in an exploratory study CD44 was knocked down in HUVECs using shRNA-containing lentiviral particles, achieving a 40% reduction in CD44 expression. This caused a ~25% decrease in total YAP levels and a 25% increase in phosphorylated YAP (S127), consistent with cytoplasmic retention and inactivation. These proof-of-concept findings further support CD44's role as a mediator of YAP activity.

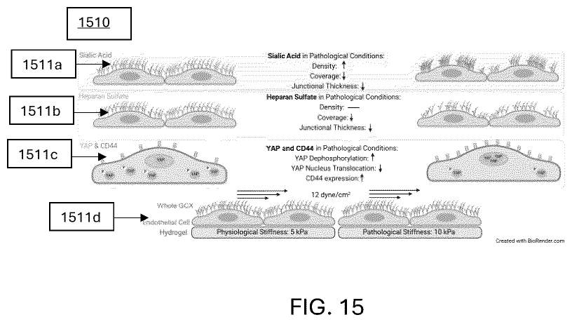

Tying together all of these observations reported herein 1510, this study investigated EC dysfunction associated with hypertension and atherosclerosis, specifically examining the impact of subendothelial substrate stiffening under constant fluid shear stress on EC GCX expression. This was first validated by the newly developed in vitro model by analyzing characteristic EC behaviors, such as cytoskeleton reorganization and YAP activity, under both healthy and diseased conditions 1511c. The primary contribution of this study lies in revealing the differential responses of GCX subcomponents to mechanical stimuli (FIG. 15). For example, pathological conditions led to increased expression of SA and CD44, while the EC coverage area and junctional thickness of HS 1511b and SA 1511a decreased (FIG. 15). These findings challenge the assumption that GCX expression universally decreases under pathological conditions, offering a more nuanced understanding of GCX behavior through this refined model. Notably, this data suggests that changes in CD44, among other GCX components, trigger YAP activation 1511d (FIGS. 14 and 15).

As mentioned earlier, the next step in this research is to compare the effects of uniform versus disturbed flow and shear stress gradients on both healthy (5 kPa) and diseased (10 kPa) subendothelial substrates, in order to better understand the GCX-mediated mechanisms driving EC dysfunction related to hypertension and atherosclerosis. Disturbed flow and shear stress gradients of interest would be those that are characteristic of hypertensive conditions. A subsequent direction for this work could involve examining the relationship between GCX components and YAP activation or testing the physiological consequences of the thinning of the GCX on barrier disruption. This could be achieved by selectively removing GCX components through RNA knockdown techniques or enzymatic degradation. Additionally, while this study focused on protein and GCX component expression, genetic changes that may require a longer time frame to translate into actual protein and GCX expression were not captured. Future work could involve RNA sequencing to investigate these genetic alterations. Furthermore, incorporating ECs other than HUVECs such as those from large arteries, along with incorporating circulating inflammatory cells and vascular smooth muscle cells within the subendothelial substrate, may enhance the model's accuracy and the reliability of the findings, providing a more comprehensive understanding of endothelial dysfunction in vascular disease.

### Conclusion

In conclusion, this study provides a foundation for exploring how mechanical stimuli, such as substrate stiffness and shear stress, influence EC behavior, particularly regarding GCX structure and function. By revealing the differential responses of GCX components in disease states, it opens avenues for targeted investigations. These findings underscore the important involvement of the GCX in vascular health, suggesting potential therapeutic targets to preserve or restore its integrity and reduce cardiovascular risks.

### REFERENCES

[1]S. S. Martin, A. W. Aday, Z. I. Almarzooq, C. A. Anderson, P. Arora, C. L. Avery, C. M. Baker-Smith, B. Barone Gibbs, A. Z. Beaton, A. K. Boehme, 2024 Heart Disease and Stroke Statistics: A Report of US and Global Data From the American Heart Association, Circulation. 149 (2024) e347-e913. https://doi.org/10.1161/CIR.0000000000001209.
[2]N. A. Shirwany, M.-h. Zou, Arterial stiffness: a brief review, Acta Pharmacologica Sinica. 31 (2010) 1267-1276. https://doi.org/10.1038/aps.2010.123.
[3]S. Jebari-Benslaiman, U. Galicia-Garcia, A. Larrea-Sebal, J. R. Olaetxea, I. Alloza, K. Vandenbroeck, A. Benito-Vicente, C. Martin, Pathophysiology of atherosclerosis, International journal of molecular sciences. 23 (2022) 3346. https://doi.org/10.3390/ijms23063346.
[4]P. Y. Chia, A. Teo, T. W. Yeo, Overview of the assessment of endothelial function in humans, Frontiers in Medicine. 7 (2020) 542567. https://doi.org/10.3389/fmed.2020.542567.
[5]Z. Peng, B. Shu, Y. Zhang, M. Wang, Endothelial response to pathophysiological stress, Arteriosclerosis, thrombosis, and vascular biology. 39 (2019) e233-e243. https://doi.org/10.1161/ATVBAHA.119.312580.
[6]C. A. Foote, R. N. Soares, F. I. Ramirez-Perez, T. Ghiarone, A. Aroor, C. Manrique-Acevedo, J. Padilla, L. A. Martinez-Lemus, Endothelial Glycocalyx, Compr Physiol. 12 (2022) 3781-3811. https://doi.org/10.1002/cphy.c210029.
[7]N. Villalba, S. Baby, S. Y. Yuan, The endothelial glycocalyx as a double-edged sword in microvascular homeostasis and pathogenesis, Frontiers in Cell and Developmental Biology. 9 (2021) 711003. https://doi.org/10.3389/fcell.2021.711003.
[8]M. Hamrangsekachaee, K. Wen, S. A. Bencherif, E. E. Ebong, Atherosclerosis and endothelial mechanotransduction: Current knowledge and models for future research, American Journal of Physiology-Cell Physiology. 324 (2023) C488-C504. https://doi.org/10.1152/ajpcell.00449.2022.
[9]S. A. Mensah, A. A. Nersesyan, E. E. Ebong, Endothelial glycocalyx-mediated intercellular interactions: mechanisms and implications for atherosclerosis and cancer metastasis, Cardiovascular engineering and technology. 12 (2021) 72-90. https://doi.org/10.1007/s13239-020-00487-7.
[10]Z. Vittum, S. Cocchiaro, S. A. Mensah, Basal endothelial glycocalyx's response to shear stress: a review of structure, function, and clinical implications, Frontiers in Cell and Developmental Biology. 12 (2024) 1371769. https://doi.org/10.3389/fcell.2024.1371769.
[11]M. Mahmoud, L. Cancel, J. M. Tarbell, Matrix Stiffness Affects Glycocalyx Expression in Cultured Endothelial Cells, Front Cell Dev Biol. 9 (2021) 731666. https://doi.org/10.3389/fcell.2021.731666.
[12]M. Hamrangsekachaee, K. Wen, N. Yazdani, R. K. Willits, S. A. Bencherif, E. E. Ebong, Endothelial glycocalyx sensitivity to chemical and mechanical sub-endothelial substrate properties, Front Bioeng Biotechnol. 11 (2023) 1250348. https://doi.org/10.3389/fbioe.2023.1250348.
[13]K. Urschel, M. Tauchi, S. Achenbach, B. Dietel, Investigation of wall shear stress in cardiovascular research and in clinical practice—from bench to bedside, International Journal of Molecular Sciences. 22 (2021) 5635. https://doi.org/10.3390/ijms22115635.
[14]H. Sawada, H. S. Lu, L. A. Cassis, A. Daugherty, Twenty years of studying AngII (angiotensin II)-induced abdominal aortic pathologies in mice: continuing questions and challenges to provide insight into the human disease, Arteriosclerosis, thrombosis, and vascular biology. 42 (2022) 277-288. https://doi.org/10.1161/ATVBAHA.121.317058.
[15]I. M. Stylianou, R. C. Bauer, M. P. Reilly, D. J. Rader, Genetic basis of atherosclerosis: insights from mice and humans, Circulation research. 110 (2012) 337-355. https://doi.org/10.1161/CIRCRESAHA.110.230854.
[16]C. M. Warboys, M. Ghim, P. D. Weinberg, Understanding mechanobiology in cultured endothelium: A review of the orbital shaker method, Atherosclerosis. 285 (2019) 170-177. https://doi.org/10.1016/j.atherosclerosis.2019.04.210.
[17]X. Wang, Y. Shen, M. Shang, X. Liu, L. L. Munn, Endothelial mechanobiology in atherosclerosis, Cardiovascular Research. 119 (2023) 1656-1675. https://doi.org/10.1093/cvr/cvad076.
[18]M. Noris, M. Morigi, R. Donadelli, S. Aiello, M. Foppolo, M. Todeschini, S. Orisio, G. Remuzzi, A. Remuzzi, Nitric oxide synthesis by cultured endothelial cells is modulated by flow conditions, Circulation research. 76 (1995) 536-543. https://doi.org/10.1161/01. RES.76.4.536.
[19]M. J. Cheng, R. Mitra, C. C. Okorafor, A. A. Nersesyan, I. C. Harding, N. N. Bal, R. Kumar, H. Jo, S. Sridhar, E. E. Ebong, Targeted Intravenous Nanoparticle Delivery: Role of Flow and Endothelial Glycocalyx Integrity, Annals of Biomedical Engineering. 48 (2020) 1941-1954. https://doi.org/10.1007/s10439-020-02474-4.
[20]S. A. Mensah, A. A. Nersesyan, I. C. Harding, C. I. Lee, X. Tan, S. Banerjee, M. Niedre, V. P. Torchilin, E. E. Ebong, Flow-regulated endothelial glycocalyx determines metastatic cancer cell activity, The FASEB Journal. 34 (2020) 6166-6184. https://doi.org/10.1096/fj.201901920R.
[21]R. Mitra, J. Qiao, S. Madhavan, G. L. O'Neil, B. Ritchie, P. Kulkarni, S. Sridhar, A. L. K. van de Ven, Erica M C, C. Ferris, J. A. Hamilton, E. E. Ebong, The comparative effects of high fat diet or disturbed blood flow on glycocalyx integrity and vascular inflammation, Translational Medicine Communications. 3 (2018). https://doi.org/10.1186/s41231-018-0029-9.
[22]V. Hosseini, A. Mallone, F. Nasrollahi, S. Ostrovidov, R. Nasiri, M. Mahmoodi, R. Haghniaz, A. Baidya, M. M. Salek, M. A. Darabi, G. Orive, A. Shamloo, M. R. Dokmeci, S. Ahadian, A. Khademhosseini, Healthy and diseased in vitro models of vascular systems, Lab on a Chip. 21 (2021) 641-659. https://doi.org/10.1039/DOLC00464B.
[23]O. Chaudhuri, J. Cooper-White, P. A. Janmey, D. J. Mooney, V. B. Shenoy, Effects of extracellular matrix viscoelasticity on cellular behaviour, Nature. 584 (2020) 535-546. https://doi.org/10.1038/s41586-020-2612-2.
[24]M. P. Lutolf, J. Hubbell, Synthetic biomaterials as instructive extracellular microenvironments for morphogenesis in tissue engineering, Nature biotechnology. 23 (2005) 47-55. https://doi.org/10.1038/nbt1055.
[25]J. Kim, S. A. Bencherif, W. A. Li, D. J. Mooney, Cell-Friendly Inverse Opal-Like Hydrogels for a Spatially Separated Co-Culture System, Macromolecular Rapid Communications. 35 (2014) 1578-1586. https://doi.org/10.1002/marc.201400278.
[26]M. Hamrangsekachaee, Y. Chen, E. R. Tressler, S. A. Bencherif, E. E. Ebong, In Vitro Model Integrating Substrate Stiffness and Flow to Study Endothelial Cell Responses, JoVE. (2024) e67081. https://doi.org/doi:10.3791/67081.
[27]I. C. Harding, N. R. O'Hare, M. Vigliotti, A. Caraballo, C. I. Lee, K. Millican, I. M. Herman, E. E. Ebong, Developing a transwell millifluidic device for studying blood-brain barrier endothelium, Lab on a Chip. 22 (2022) 4603-4620. https://doi.org/10.1039/d21c00657j.
[28]S. Dupont, L. Morsut, M. Aragona, E. Enzo, S. Giulitti, M. Cordenonsi, F. Zanconato, J. Le Digabel, M. Forcato, S. Bicciato, N. Elvassore, S. Piccolo, Role of YAP/TAZ in mechanotransduction, Nature. 474 (2011) 179-183. https://doi.org/10.1038/nature10137.
[29]M. Mahmoud, M. Mayer, L. M. Cancel, A. M. Bartosch, R. Mathews, J. M. Tarbell, The glycocalyx core protein Glypican 1 protects vessel wall endothelial cells from stiffness-mediated dysfunction and disease, Cardiovasc Res. 117 (2021) 1592-1605. https://doi.org/10.1093/cvr/cvaa201.
[30]A. Yoneda, J. R. Couchman, Regulation of cytoskeletal organization by syndecan transmembrane proteoglycans, Matrix Biology. 22 (2003) 25-33. https://doi.org/10.1016/S0945-053X(03)00010-6.
[31]K. Zhang, Y. Chen, T. Zhang, L. Huang, Y. Wang, T. Yin, J. Qiu, H. Gregersen, G. Wang, A Novel Role of Id1 in Regulating Oscillatory Shear Stress-Mediated Lipid Uptake in Endothelial Cells, Annals of Biomedical Engineering. 46 (2018) 849-863. https://doi.org/10.1007/s10439-018-2000-3.
[32]X. Qin, K. Zhang, J. Qiu, N. Wang, K. Qu, Y. Cui, J. Huang, L. Luo, Y. Zhong, T. Tian, W. Wu, Y. Wang, G. Wang, Uptake of oxidative stress-mediated extracellular vesicles by vascular endothelial cells under low magnitude shear stress, Bioactive Materials. 9 (2022) 397-410. https://doi.org/10.1016/j.bioactmat.2021.10.038.
[33]A. Pocaterra, P. Romani, S. Dupont, YAP/TAZ functions and their regulation at a glance, J Cell Sci. 133 (2020). https://doi.org/10.1242/jcs.230425.
[34]H. Nakajima, K. Yamamoto, S. Agarwala, K. Terai, H. Fukui, S. Fukuhara, K. Ando, T. Miyazaki, Y. Yokota, E. Schmelzer, H. G. Belting, M. Affolter, V. Lecaudey, N. Mochizuki, Flow-Dependent Endothelial YAP Regulation Contributes to Vessel Maintenance, Dev Cell. 40 (2017) 523-536 e526. https://doi.org/10.1016/j.devcel.2017.02.019.
[35]S. Dupont, L. Morsut, M. Aragona, E. Enzo, S. Giulitti, M. Cordenonsi, F. Zanconato, J. Le Digabel, M. Forcato, S. Bicciato, N. Elvassore, S. Piccolo, Role of YAP/TAZ in mechanotransduction, Nature. 474 (2011) 179-183. https://doi.org/10.1038/nature10137.
[36]Y. T. Yeh, S. S. Hur, J. Chang, K. C. Wang, J. J. Chiu, Y. S. Li, S. Chien, Matrix stiffness regulates endothelial cell proliferation through septin 9, PLoS One. 7 (2012) e46889. https://doi.org/10.1371/journal.pone.0046889.
[37]E. S. Fioretta, J. O. Fledderus, F. P. T. Baaijens, C. V. C. Bouten, Influence of substrate stiffness on circulating progenitor cell fate, Journal of Biomechanics. 45 (2012) 736-744. https://doi.org/10.1016/j.jbiomech.2011.11.013.
[38]D. v. Geemen, M. W. J. Smeets, A.-M. D. v. Stalborch, L. A. E. Woerdeman, M. J. A. P. Daemen, P. L. Hordijk, S. Huveneers, F-Actin-Anchored Focal Adhesions Distinguish Endothelial Phenotypes of Human Arteries and Veins, Arteriosclerosis, Thrombosis, and Vascular Biology. 34 (2014) 2059-2067. https://doi.org/10.1161/ATVBAHA.114.304180.
[39]C. A. Dessalles, C. Leclech, A. Castagnino, A. I. Barakat, Integration of substrate- and flow-derived stresses in endothelial cell mechanobiology, Communications Biology. 4 (2021) 764. https://doi.org/10.1038/s42003-021-02285-w.
[40]R. Estrada, G. A. Giridharan, M.-D. Nguyen, S. D. Prabhu, P. Sethu, Microfluidic endothelial cell culture model to replicate disturbed flow conditions seen in atherosclerosis susceptible regions, Biomicrofluidics. 5 (2011). https://doi.org/10.1063/1.3608137.
[41]M. Inglebert, L. Locatelli, D. Tsvirkun, P. Sinha, J. A. Maier, C. Misbah, L. Bureau, The effect of shear stress reduction on endothelial cells: A microfluidic study of the actin cytoskeleton, Biomicrofluidics. 14 (2020) 024115. https://doi.org/10.1063/1.5143391.
[42]S. Noria, F. Xu, S. McCue, M. Jones, A. I. Gotlieb, B. L. Langille, Assembly and reorientation of stress fibers drives morphological changes to endothelial cells exposed to shear stress, Am J Pathol. 164 (2004) 1211-1223. https://doi.org/10.1016/s0002-9440(10)63209-9.
[43]P. Belvitch, Y. M. Htwe, M. E. Brown, S. Dudek, Cortical Actin Dynamics in Endothelial Permeability, Curr Top Membr. 82 (2018) 141-195. https://doi.org/10.1016/bs.ctm.2018.09.003.
[44]M. Aragona, T. Panciera, A. Manfrin, S. Giulitti, F. Michielin, N. Elvassore, S. Dupont, S. Piccolo, A mechanical checkpoint controls multicellular growth through YAP/TAZ regulation by actin-processing factors, Cell. 154 (2013) 1047-1059. https://doi.org/10.1016/j.cell.2013.07.042.
[45]H. J. Choi, H. Zhang, H. Park, K. S. Choi, H. W. Lee, V. Agrawal, Y. M. Kim, Y. G. Kwon, Yes-associated protein regulates endothelial cell contact-mediated expression of angiopoietin-2, Nat Commun. 6 (2015) 6943. https://doi.org/10.1038/ncomms7943.
[46]F. Neto, A. Klaus-Bergmann, Y. T. Ong, S. Alt, A. C. Vion, A. Szymborska, J. R. Carvalho, I. Hollfinger, E. Bartels-Klein, C. A. Franco, M. Potente, H. Gerhardt, YAP and TAZ regulate adherens junction dynamics and endothelial cell distribution during vascular development, Elife. 7 (2018). https://doi.org/10.7554/eLife.31037.
[47]A. Elosegui-Artola, I. Andreu, A. E. M. Beedle, A. Lezamiz, M. Uroz, A. J. Kosmalska, R. Oria, J. Z. Kechagia, P. Rico-Lastres, A. L. Le Roux, C. M. Shanahan, X. Trepat, D. Navajas, S. Garcia-Manyes, P. Roca-Cusachs, Force Triggers YAP Nuclear Entry by Regulating Transport across Nuclear Pores, Cell. 171 (2017) 1397-1410 e1314. https://doi.org/10.1016/j.cell.2017.10.008.
[48]I. C. Harding, R. Mitra, S. A. Mensah, I. M. Herman, E. E. Ebong, Pro-atherosclerotic disturbed flow disrupts caveolin-1 expression, localization, and function via glycocalyx degradation, Journal of Translational Medicine. 16 (2018) 364. https://doi.org/10.1186/s12967-018-1721-2.
[49]S. M. Opal, C. M. Kessler, J. Roemisch, S. Knaub, Antithrombin, heparin, and heparan sulfate, Critical Care Medicine. 30 (2002) 5325-5331. https://doi.org/10.1097/00003246-200205001-00024.
[50]B. Baker Aaron, J. Gibson William, B. Kolachalama Vijaya, M. Golomb, L. Indolfi, C. Spruell, E. Zcharia, I. Vlodavsky, R. Edelman Elazer, Heparanase Regulates Thrombosis in Vascular Injury and Stent-Induced Flow Disturbance, Journal of the American College of Cardiology. 59 (2012) 1551-1560. https://doi.org/10.1016/j.jacc.2011.11.057.
[51]I. S. Fancher, E. Le Master, S. J. Ahn, C. Adamos, J. C. Lee, E. Berdyshev, R. O. Dull, S. A. Phillips, I. Levitan, Impairment of Flow-Sensitive Inwardly Rectifying K(+) Channels via Disruption of Glycocalyx Mediates Obesity-Induced Endothelial Dysfunction, Arterioscler Thromb Vasc Biol. 40 (2020) e240-e255. https://doi.org/10.1161/ATVBAHA.120.314935.
[52]A. W. Mulivor, H. H. Lipowsky, Role of glycocalyx in leukocyte-endothelial cell adhesion, American Journal of Physiology-Heart and Circulatory Physiology. 283 (2002) H1282-H1291. https://doi.org/10.1152/ajpheart.00117.2002.
[53]M. A. Stepp, S. Pal-Ghosh, G. Tadvalkar, A. Pajoohesh-Ganji, Syndecan-1 and Its Expanding List of Contacts, Advances in Wound Care. 4 (2014) 235-249. https://doi.org/10.1089/wound.2014.0555.
[54]J. K. Langford, M. J. Stanley, D. Cao, R. D. Sanderson, Multiple Heparan Sulfate Chains Are Required for Optimal Syndecan-1 Function *, Journal of Biological Chemistry. 273 (1998) 29965-29971. https://doi.org/10.1074/jbc.273.45.29965.
[55]A. Varki, P. Gagneux, Multifarious roles of sialic acids in immunity, Annals of the New York Academy of Sciences. 1253 (2012) 16-36. https://doi.org/10.1111/j.1749-6632.2012.06517.x.
[56]M. D′Addio, J. Frey, V. I. Otto, The manifold roles of sialic acid for the biological functions of endothelial glycoproteins, Glycobiology. 30 (2020) 490-499. https://doi.org/10.1093/glycob/cwaa008.
[57]A. Chandrasekaran, A. Srinivasan, R. Raman, K. Viswanathan, S. Raguram, T. M. Tumpey, V. Sasisekharan, R. Sasisekharan, Glycan topology determines human adaptation of avian H5N1 virus hemagglutinin, Nature Biotechnology. 26 (2008) 107-113. https://doi.org/10.1038/nbt1375.
[58]S. Kitazume, R. Imamaki, K. Ogawa, Y. Komi, S. Futakawa, S. Kojima, Y. Hashimoto, J. D. Marth, J. C. Paulson, N. Taniguchi, α2,6-Sialic Acid on Platelet Endothelial Cell Adhesion Molecule (PECAM) Regulates Its Homophilic Interactions and Downstream Antiapoptotic Signaling, Journal of Biological Chemistry. 285 (2010) 6515-6521. https://doi.org/10.1074/jbc. M109.073106.
[59]E. Rijcken, R. B. Mennigen, S. D. Schaefer, M. G. Laukoetter, C. Anthoni, H.-U. Spiegel, M. Bruewer, N. Senninger, C. F. Krieglstein, PECAM-1 (CD 31) mediates transendothelial leukocyte migration in experimental colitis, American Journal of Physiology-Gastrointestinal and Liver Physiology. 293 (2007) G446-G452. https://doi.org/10.1152/ajpgi.00097.2007.
[60]W. A. Muller, S. A. Weigl, X. Deng, D. M. Phillips, PECAM-1 is required for transendothelial migration of leukocytes, J Exp Med. 178 (1993) 449-460. https://doi.org/10.1084/jem.178.2.449.
[61]A. Woodfin, M. B. Voisin, B. A. Imhof, E. Dejana, B. Engelhardt, S. Nourshargh, Endothelial cell activation leads to neutrophil transmigration as supported by the sequential roles of ICAM-2, JAM-A, and PECAM-1, Blood. 113 (2009) 6246-6257. https://doi.org/10.1182/blood-2008-11-188375.
[62]J. R. Privratsky, D. K. Newman, P. J. Newman, PECAM-1: conflicts of interest in inflammation, Life Sci. 87 (2010) 69-82. https://doi.org/10.1016/j.lfs.2010.06.001.
[63]B. L. Harry, J. M. Sanders, R. E. Feaver, M. Lansey, T. L. Deem, A. Zarbock, A. C. Bruce, A. W. Pryor, B. D. Gelfand, B. R. Blackman, M. A. Schwartz, K. Ley, Endothelial Cell PECAM-1 Promotes Atherosclerotic Lesions in Areas of Disturbed Flow in ApoE-Deficient Mice, Arteriosclerosis, Thrombosis, and Vascular Biology. 28 (2008) 2003-2008. https://doi.org/doi:10.1161/ATVBAHA.108.164707.
[64]K. M. A. Rouschop, M. E. Sewnath, N. Claessen, J. J. T. H. Roelofs, I. Hoedemaeker, R. van der Neut, J. Aten, S. T. Pals, J. J. Weening, S. Florquin, CD44 Deficiency Increases Tubular Damage But Reduces Renal Fibrosis in Obstructive Nephropathy, Journal of the American Society of Nephrology. 15 (2004) 674-686. https://doi.org/10.1097/01. Asn.0000115703.30835.96.
[65]G. Wang, G. L. Tiemeier, B. M. van den Berg, T. J. Rabelink, Endothelial Glycocalyx Hyaluronan: Regulation and Role in Prevention of Diabetic Complications, The American Journal of Pathology. 190 (2020) 781-790. https://doi.org/10.1016/j.ajpath.2019.07.022.
[66]N. Nagy, T. Freudenberger, A. Melchior-Becker, K. Röck, M. t. Braak, H. Jastrow, M. Kinzig, S. Lucke, T. Suvorava, G. Kojda, A. A. Weber, F. Sörgel, B. Levkau, S. Ergün, J. W. Fischer, Inhibition of Hyaluronan Synthesis Accelerates Murine Atherosclerosis, Circulation. 122 (2010) 2313-2322. https://doi.org/10.1161/CIRCULATIONAHA.110.972653.
[67]L. Zhao, J. A. Hall, N. Levenkova, E. Lee, M. K. Middleton, A. M. Zukas, D. J. Rader, J. J. Rux, E. Puré, CD44 Regulates Vascular Gene Expression in a Proatherogenic Environment, Arteriosclerosis, Thrombosis, and Vascular Biology. 27 (2007) 886-892. https://doi.org/10.1161/01. ATV.0000259362.10882.c5.
[68]C. A. Cuff, D. Kothapalli, I. Azonobi, S. Chun, Y. Zhang, R. Belkin, C. Yeh, A. Secreto, R. K. Assoian, D. J. Rader, E. Puré, The adhesion receptor CD44 promotes atherosclerosis by mediating inflammatory cell recruitment and vascular cell activation, The Journal of Clinical Investigation. 108 (2001) 1031-1040. https://doi.org/10.1172/JCI12455.
[69]L. Zhao, E. Lee, A. M. Zukas, M. K. Middleton, M. Kinder, P. S. Acharya, J. A. Hall, D. J. Rader, E. Puré, CD44 Expressed on Both Bone Marrow-Derived and Non-Bone Marrow-Derived Cells Promotes Atherogenesis in ApoE-Deficient Mice, Arteriosclerosis, Thrombosis, and Vascular Biology. 28 (2008) 1283-1289. https://doi.org/10.1161/ATVBAHA.108.165753.
[70]D. Hagg, S. Sjöberg, L. M. Hultén, B. Fagerberg, O. Wiklund, A. Rosengren, L. M. S. Carlsson, J. Borén, P.-A. Svensson, A. Krettek, Augmented levels of CD44 in macrophages from atherosclerotic subjects: A possible IL-6-CD44 feedback loop?, Atherosclerosis. 190 (2007) 291-297. https://doi.org/10.1016/j.atherosclerosis.2006.03.020.
[71]A. Krettek, G. K. Sukhova, U. Schönbeck, P. Libby, Enhanced Expression of CD44 Variants in Human Atheroma and Abdominal Aortic Aneurysm: Possible Role for a Feedback Loop in Endothelial Cells, The American Journal of Pathology. 165 (2004) 1571-1581. https://doi.org/10.1016/S0002-9440(10)63414-1.
[72]Z. Fan, H. Xia, H. Xu, J. Ma, S. Zhou, W. Hou, Q. Tang, Q. Gong, Y. Nie, F. Bi, Standard CD44 modulates YAP1 through a positive feedback loop in hepatocellular carcinoma, Biomedicine & Pharmacotherapy. 103 (2018) 147-156. https://doi.org/10.1016/j.biopha.2018.03.042.
[73]C.-J. Lai, C.-Y. Lin, W.-Y. Liao, T.-C. Hour, H.-D. Wang, C.-P. Chuu, CD44 Promotes Migration and Invasion of Docetaxel-Resistant Prostate Cancer Cells Likely via Induction of Hippo-Yap Signaling, Cells. 8 (2019) 295. https://doi.org/10.3390/cells8040295.
[74]X. Chen, G. Adhikary, E. Ma, J. J. Newland, W. Naselsky, W. Xu, R. L. Eckert, Sulforaphane inhibits CD44v6/YAP1/TEAD signaling to suppress the cancer phenotype, Molecular Carcinogenesis. 62 (2023) 236-248. https://doi.org/10.1002/mc.23479.
[75]S. Li, C. Li, Y. Zhang, X. He, X. Chen, X. Zeng, F. Liu, Y. Chen, J. Chen, Targeting Mechanics-Induced Fibroblast Activation through CD44-RhoA-YAP Pathway Ameliorates Crystalline Silica-Induced Silicosis, Theranostics. 9 (2019) 4993-5008. https://doi.org/10.7150/thno.35665.

The teachings of all patents, published applications and references cited herein or in the manuscripts being filed herewith are incorporated by reference in their entirety.

While example embodiments have been particularly shown and described, it will be understood by those skilled in the art that various changes in form and details may be made therein without departing from the scope of the embodiments encompassed or contemplated herein or in the manuscripts being filed herewith.

We claim:

1. An apparatus for performing an in vitro study of a blood vessel function, the apparatus comprising:
a flow chamber structure defining an inlet port, outlet port, flowpath therebetween, and coupling feature; and
a hydrogel, seeded with cells, coupled to the coupling feature, the hydrogel, in its coupled arrangement, positioned within the flowpath, a combination of characteristics of the hydrogel and dimensions of the flowpath in a presence of fluid flow creating a pressure between the inlet port and outlet port that causes the apparatus to emulate a blood vessel function.

2. The apparatus of claim 1, wherein the flow chamber structure comprises:
a) a flow chamber top substructure defining the inlet port and the outlet port;
b) a flow chamber middle substructure defining at least one window;
c) a flow chamber gasket defining at least one well therein; and
d) a flow chamber bottom substructure;
wherein the flow chamber top substructure, flow chamber middle substructure, flow chamber gasket, and flow chamber bottom substructure define respective holes that, in stacked arrangement of the substructures, are aligned; and, in an assembled arrangement, the flow chamber structure produces within the flowpath a uniform laminar flow of fluid during operation with an operating fluid flow pump.

3. The apparatus of claim 2, wherein the number of flow chamber middle windows and number of hydrogels are of equal number.

4. The apparatus of claim 1, wherein the cells are embedded in the hydrogel.

5. The apparatus of claim 1, wherein the cells are endothelial or epithelial cells.

6. The apparatus of claim 5, wherein the endothelial or epithelial cells produce a layer of glycocalyx (GCX) that cover at least a portion of the hydrogel.

7. The apparatus of claim 6, wherein the flow chamber structure emulates pathological or physiological blood vessels by exposing the layer of GCX to a uniform laminar flow during operation with a fluid flow pump.

8. The apparatus of claim 1, wherein the hydrogel comprises gelatin methacrylate (GelMA) and has an exterior layer of gelatin surrounding the GelMA.

9. The apparatus of claim 1, wherein the hydrogel has a stiffness from about 2.5 kPa to about 10 kPa.

10. The apparatus of claim 1 further comprising:
a) a flow pump configured to pump fluid, the flow pump having an inlet port and an outlet port; and
b) flow tubing having dimensions that enable the flow tubing to couple the outlet port of the flow pump to the inlet port of the flow chamber and the outlet port of the flow chamber to the inlet port of the flow pump, a combination of the flow pump, flow chamber, and flow tubing defining a fluid flow loop that supports the fluid flow.

11. The apparatus of claim 10, wherein the hydrogel comprises gelatin methacrylate (GelMA) and has at least a portion of an exterior layer of gelatin surrounding the GelMA.

12. The apparatus of claim 10, wherein the hydrogel has a stiffness from about 2.5 kPa to about 10 kPa.

13. The apparatus of claim 10, wherein the apparatus, during operation, emulates a physiological blood vessel function.

14. The apparatus of claim 10, wherein the apparatus, during operation, emulates a pathological blood vessel function.

15. The apparatus of claim 10, wherein the flow pump is configurable to start pumping at an initial shear stress of about 3.6 dyne/cm² and gradually increase the shear stress to a final shear stress of about 12 dyne/cm².

16. The apparatus of claim 10, wherein the endothelial or epithelial cells have an apical surface between the cells and a GCX layer, a basal surface between the cells and the hydrogel, and a junctional surface where the GCX layer is on the hydrogel.

17. A method for performing an in vitro study of a blood vessel function, the method comprising:
retaining a hydrogel, seeded with cells, within a flowpath; and
causing a fluid to flow in the flowpath to form a fluid flow that interacts with the hydrogel, a combination of characteristics of the hydrogel and dimensions of the flowpath in a presence of the fluid flow creating a pressure that causes the method to emulate a blood vessel function.

18. The method of claim 17, wherein the in vitro study enables testing of a disease comprising cardiovascular disease, infection, inflammation, cancer metastasis, or a combination thereof.

19. The method of claim 17, wherein the cardiovascular disease is atherosclerosis or hypertension.

20. The method of claim 17, wherein the in vitro study comprises screening therapeutic agents in the fluid flow for cell targeting, treatment for a disease, or a combination of screening for cell targeting and treatment for a disease by comparing a control sample and a diseased cell, or sample comprising a diseased cell, or by comparing a cell sample before treatment and after treatment.

21. The method of claim 20, wherein the fluid flow uses a medium comprising cancer cells in the fluid that forms the fluid flow in the flowpath.

22. A kit comprising:
a) an apparatus for performing an in vitro study of a blood vessel function, the apparatus comprising a flow chamber structure defining an inlet port, outlet port, flowpath therebetween, and coupling feature; and
b) a substrate defining features that cause a hydrogel formed thereon to express at least one feature that enables the hydrogel, in a formed state, to couple to the coupling feature in a manner that, in a coupled arrangement, enables the hydrogel to be positioned within the flowpath, a combination of characteristics of the hydrogel and dimensions of the flowpath create a pressure between the inlet port and outlet port that causes the apparatus to emulate a blood vessel function in a presence of fluid flow.
